(19) United States
(12) Patent Application Publication
(10) Pub. No.: US 2026/0365604 A1
(43) Pub. Date: Nov. 19, 2026

(54) A MATERIAL COMPRISING GEMINAL METAL ATOM PAIRS SUPPORTED ON A CARRIER AND RELATED METHODS THERETO

(71) Applicant: National University of Singapore, Singapore (SG)

(72) Inventors: Jiong Lu, Singapore (SG); Xiao Hai, Singapore (SG)

(21) Appl. No.: 19/107,827

(22) PCT Filed: Aug. 29, 2023
(86) PCT No.: PCT/SG2023/050592

§ 371 Date: Feb. 28, 2025

(30) Foreign Application Priority Data
Sep. 2, 2022 (SG) 10202250888X

(51) Int. Cl. C07C 41/09; B01J 31/06; B01J 37/30; C07D 233/58; C07D 233/60; C08G 73/06; C08K 3/08

(52) U.S. Cl. CPC C07C 41/09; B01J 31/06; B01J 37/30; C07D 233/58; C07D 233/60; C08G 73/0683; C08K 3/08; B01J 2231/324; B01J 2231/4205; B01J 2231/4283; B01J 2231/4288; B01J 2531/005; B01J 2531/0216; B01J 2531/16; C08K 2003/085

(57) ABSTRACT

There is provided a material comprising a plurality of geminal metal atom pairs supported on a polymeric carrier, and a method of preparing said material. In a particular embodiment, the material comprises a plurality of copper atom pairs supported on a polymeric carbon nitride (PCN) carrier. There is also provided a catalyst comprising said material, and a method of catalyzing coupling reactions.

### TECHNICAL FIELD

The present disclosure relates broadly to a material comprising geminal metal atom pairs supported on a carrier and a method of preparing said material. The present disclosure also relates to a catalyst comprising said material and a method of catalyzing coupling reactions.

### BACKGROUND

Homogeneous organometallic complexes are typically exploited for transition-metal-catalyzed reactions (e.g., cross coupling reactions) which are critical for developing molecular complexity in organic syntheses. Despite their unparalleled synthetic capability, there are numerous concerns arising from using homogeneous catalysts which include their potentially high production costs, the environmental impact associated with challenges in product separation and purification, catalyst recycling, and operation under harsh reaction conditions.

Turning to heterogeneous catalytic systems, the development of heterogeneously-catalyzed processes has been highly attractive for large-scale production to facilitate catalyst separation, recovery and reuse, and improve adaptability for continuous flow synthesis. These potential advantages have triggered extensive studies of immobilized organometallic complexes and nanostructured metal catalysts. However, although some have been successfully implemented industrially, their applicability in reactions (e.g., cross-coupling reactions) remains limited, primarily owing to poor control of active site structures or weak interactions with supports.

Heterogeneous single-atom catalysts (SACs), which integrate well-defined mononuclear metal sites have sparked interest for their potential to overcome the shortcomings of previously developed catalytic solids. Particularly, the presence of well-defined active sites in single-atom catalysts (SACs) triggers interest in their potential for heterogeneously-catalyzed organic synthesis. However, the architecture of mononuclear metal species stabilized on solid supports may not be optimal in catalyzing complex molecular transformations due to restricted spatial environment and electronic quantum states. It is believed that the carrier design must ensure the stability of metal centres while permitting structural flexibility to fulfil the catalytic cycle and achieve enzyme-like specific activity. Nonetheless, the chemical bonding between the metal centre and the carrier required to prevent metal detachment or aggregation typically leads to restricted spatial environment (e.g., high coordination numbers), limiting its capacity to activate complicated or multiple substrates simultaneously. These seemingly conflicting requirements have prompted debate over whether mononuclear metal sites provide the optimal architecture for complex molecular transformations. Some researchers postulated that tailored multinuclear sites may be more efficient, but controlled synthetic routes for other low-nuclearity catalysts remain limited.

An alternative approach to exploit cooperativity between metal centres in SACs by controlling their spatial proximity has been investigated (FIG. 14). For example, many researchers have studied the performance of nanostructured metal catalysts (e.g., supported metallic or oxidic metal clusters or nanoparticles, immobilized metal complexes, and metal-organic frameworks (MOFs) or zeolites) in cross-coupling applications. However, despite the fact that some materials have been commercialized, all of these classes of catalyst have intrinsic limitations that hinder their broad industrial application. Specifically, supported metal-based clusters or nanoparticles may contain inaccessible metal atoms in the bulk of the structure leading to poor metal utilization, furthermore the lack of atomic control in their structure and resulting diversity of active sites limits their selectivity and also makes it very challenging to understand the reaction mechanism. Since metal atoms in extended metal surfaces may strongly interact with reaction intermediates and ligands in coupling applications, high levels of metal leaching are common, which has triggered extensive debate over whether the observed performance is heterogeneously or homogeneously catalysed. MOFs and zeolites have intrinsic problems associated with poor accessibility of active sites within microporous channels, high costs of the support materials, and strong adsorption of organic components in the case of zeolites and poor structural stability in the case of MOFs. Immobilized metal complexes operate under a similar principle to organometallic complexes, meaning that the ligands in the structure are removed and replaced during the catalytic cycle. This implies that the metal centres can have a very low coordination number to the solid carrier and therefore a high tendency to leach into the reaction mixture. Heterogeneous single-atom catalysts (SACs) with well-defined metal sites have attracted growing attention due to their potential to bridge homogeneous and heterogeneous catalysis for fine chemical production. However, the strong interaction between single-metal centres and the carrier necessary to prevent metal detachment or aggregation typically require high coordination numbers, rendering them inactive because of their limited capacity to activate multiple substrates simultaneously. These limitations call for technology innovation in the design and synthesis of new efficient heterogeneous catalysts that are active enough but also stable against leaching for organic coupling reactions.

In view of the above, there is a need to address or at least ameliorate the above-mentioned problems. In particular, there is a need to provide a material comprising geminal metal atom pairs supported on a carrier, a catalyst comprising said material and related methods thereto that address or at least ameliorate the above-mentioned problems.

### SUMMARY

In one aspect, there is provided a material comprising a plurality of geminal metal atom pairs supported on a polymeric carrier.

In one embodiment, each geminal metal atom pair comprises two single metal atoms that are not bonded to each other.

In one embodiment, the two single metal atoms are each bound to different metal coordination site(s) of the polymeric carrier.

In one embodiment, an average distance between the two single metal atoms is from 1.0 Å to 10.0 Å.

In one embodiment, each single metal atom is diagonally bound to two different metal coordination sites of the polymeric carrier.

In one embodiment, the material comprises a structure represented by general formula (1); and a structure represented by general formula (2):

-A¹-M¹-A²- (1)

-A³-M²-A⁴- (2)
wherein
M¹ and M² are each independently a single metal atom;
M¹ and M² both form a pair of geminal metal atoms; and
A¹, A², A³ and A⁴ are each independently a metal coordination site of the polymeric carrier.

In one embodiment, the material comprises from 0.1 wt % to 50.0 wt % of metal.

In one embodiment, the metal comprises transition metal selected from the group consisting of copper (Cu), zinc (Zn), nickel (Ni), cobalt (Co), iron (Fe), manganese (Mn), chromium (Cr), vanadium (V), titanium (Ti), scandium (Sc), yttrium (Y), zirconium (Zr), niobium (Nb), molybdenum (Mo), technetium (Tc), ruthenium (Ru), rhodium (Rh), palladium (Pd), silver (Ag), cadmium (Cd), lanthanum (La), hafnium (Hf), tantalum (Ta), tungsten (W), rhenium (Re), osmium (Os), iridium (Ir), platinum (Pt), gold (Au), mercury (Hg), actinium (Ac), and combinations thereof.

In one embodiment, the metal is monovalent.

In one embodiment, the polymeric carrier comprises an organic polymer comprising one or more atoms selected from the group consisting of carbon, nitrogen, phosphorus, sulfur, and combinations thereof.

In one embodiment, the polymeric carrier comprises polymeric chains having two or more fused triazine rings.

In one embodiment, the material comprises a first shell comprising the metal atom and a first atom of the polymeric carrier.

In one embodiment, the interatomic distance between the metal atom and the first atom of the polymeric carrier in the first shell is from 0.1 Å to 5.0 Å.

In one embodiment, the material comprises a second shell comprising the metal atom and a second atom of the polymeric carrier.

In one embodiment, the interatomic distance between the metal atom and the second atom of the polymeric carrier in the second shell is from 1.5 Å to 5.0 Å.

In one aspect, there is provided a catalyst comprising the material disclosed herein.

In one aspect, there is provided a method of catalyzing coupling reactions, the method comprising:
(a-i) providing the material disclosed herein; and
(a-ii) mixing said material with two or more reactants to be coupled to obtain a coupled product.

In one embodiment, the coupling reactions comprise cross-coupling reaction, cross-coupling bond formation, cycloaddition, carbon-carbon bond formation, carbon-heteroatom bond formation, N-arylation or combinations thereof.

In one embodiment, the reactants are selected from the group consisting of heteroaryl halides, aryl halides, substrates containing hydroxyl group(s), substrates containing amino group(s), substrates containing amide group(s), substrates containing sulfonamide group(s), substrates containing alkyne group(s), substrates containing thiol group(s), substrates containing electron rich aryl group(s), substrates containing electron deficient aryl group(s), alcohols, halogenated alcohols, deuterated alcohols, heterocyclic compounds, alkynes, thiols, azides, acetylenes, L-menthol, DL-menthol, stigmasterol, DL-isoborneol, galactose, 1,2,3,4-diacetone galactose, testosterone, prednisolone, derivatives thereof and combinations thereof.

In one aspect, there is provided a method of preparing the material disclosed herein, the method comprising:
(i) reacting a metal source with a polymeric carrier to obtain the material; and
(ii) isolating the material.

In one embodiment, the method further comprises a step of exfoliating the material into nanostructures.

In one embodiment, the metal source comprises a metal salt selected from the group consisting of metal chloride, metal fluoride, metal bromide, metal iodide, metal oxide, metal sulfate, metal sulfide, metal cyanide, metal thiocyanate, metal acetylide, metal acetate, metal hydroxide, metal hydride, metal nitrate, metal phosphide, metal phosphate, metal carbonate, metal carbonate hydroxide, metal chlorate, metal arsenate, metal azide, metal acetylacetonate, metal perchlorate, metal triflate, metal tetrafluoroborate, metal benzoate, metal chromite, metal peroxide, and combinations thereof.

In one embodiment, the metal source comprises a divalent metal cation.

In one embodiment, the reacting step comprises adding from 0.1 wt % to 50.0 wt % of the metal source.

In one embodiment, the reacting step comprises adding from 50.0 wt % to 99.9 wt % of the polymeric carrier.

### DEFINITIONS

The term “carrier” as used herein is to be interpreted broadly to refer to any substance that provides one or more site(s) for supporting/anchoring species, atoms, molecules, ions or particles etc. The site(s) may be an active site(s) for supporting/anchoring metal species, atoms, molecules, ions or particles. The carrier may be a non-metal carrier. Examples of carrier include polymers, co-polymers, block polymers, microparticles, nanoparticles, solid surfaces and the like.

The term “alkyl” as a group or part of a group refers to a straight or branched aliphatic hydrocarbon group having 1 to 20 carbon atoms, 1 to 10 carbon atoms, 1 to 6 carbon atoms, or 1, 2, 3, 4, 5, 6, 7, 8, 9, 10, 11, 12, 13, 14, 15, 16, 17, 18, 19 or 20 carbon atoms. Examples of suitable straight and branched alkyl substituents include methyl, ethyl, n-propyl, 2-propyl, isopropyl, n-butyl, isobutyl, sec-butyl, t-butyl, hexyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, 2-ethylpentyl, 3-ethylpentyl, heptyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, 5-methylheptyl, 1-methylheptyl, octyl, nonyl, decyl and the like. The group may be a terminal group or a bridging group.

The term “alkenyl” as a group or part of a group denotes an aliphatic hydrocarbon group containing at least one carbon-carbon double bond and which may be straight or branched having 2 to 20 carbon atoms, 2 to 10 carbon atoms, 2 to 6 carbon atoms, or 2, 3, 4, 5, 6, 7, 8, 9, 10, 11, 12, 13, 14, 15, 16, 17, 18, 19 or 20 carbon atoms in the chain. The group may contain a plurality of double bonds and the orientation about each double bond is independently E or Z. Exemplary alkenyl groups include, but are not limited to, ethenyl, vinyl, allyl, 1-methylvinyl, 1-propenyl, 2-propenyl, 2-methyl-1-propenyl, 2-methyl-1-propenyl, 1-butenyl, 2-butenyl, 3-butentyl, 1,3-butadienyl, 1-pentenyl, 2-pententyl, 3-pentenyl, 4-pentenyl, 1,3-pentadienyl, 2,4-pentadienyl, 1,4-pentadienyl, 3-methyl-2-butenyl, 1-hexenyl, 2-hexenyl, 3-hexenyl, 1,3-hexadienyl, 1,4-hexadienyl, 2-methylpentenyl, 1-heptenyl, 2-heptentyl, 3-heptenyl, 1-octenyl, 2-octenyl, 3-octenyl, 1-nonenyl, 2-nonenyl, 3-nonenyl, 1-decenyl, 2-decenyl, 3-decenyl and the like. The group may be a terminal group or a bridging group.

The term “alkynyl” as a group or part of a group denotes an aliphatic hydrocarbon group containing at least one carbon-carbon triple bond and which may be straight or branched having 2 to 20 carbon atoms, 2 to 10 carbon atoms, 2 to 6 carbon atoms, or 2, 3, 4, 5, 6, 7, 8, 9, 10, 11, 12, 13, 14, 15, 16, 17, 18, 19 or 20 carbon atoms in the chain. The group may contain a plurality of triple bonds. Exemplary alkynyl groups include, but are not limited to, acetylenyl, propynyl, 1-butynyl, 2-butynyl, 3-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl, 9-decynyl and the like. The group may be a terminal group or a bridging group.

The term “aryl” as a group or part of a group denotes (i) an optionally substituted monocyclic, or fused polycyclic, aromatic carbocycle (ring structure having ring atoms that are all carbon) preferably having from 5 to 20, or 5, 6, 7, 8, 9, 10, 11, 12, 13, 14, 15, 16, 17, 18, 19 or 20 carbon atoms per ring. Examples of aryl groups include but are not limited to phenyl, tolyl, xylyl, naphthyl, anthracenyl, phenanthrenyl, fluorenyl, indenyl or indanyl and the like.

The term “heteroaryl” as a group or part of a group refers to groups containing an aromatic ring (preferably a 5- or 6-membered aromatic ring) having one or more carbon atoms (for example 1 to 6 carbon atoms) in the ring replaced by a heteroatom. Suitable heteroatoms may include nitrogen (N) or (NH), oxygen (O) and sulfur (S). Examples of heteroaryl include but are not limited to thiophene, benzothiophene, benzofuran, benzimidazole, benzoxazole, benzothiazole, benzisothiazole, naphtha[2,3-b]thiophene, furan, isoindolizine, xantholene, phenoxatine, pyrrole, imidazole, pyrazole, pyridine, pyrazine, pyrimidine, pyridazine, tetrazole, indole, isoindole, 1H-indazole, purine, quinoline, isoquinoline, phthalazine, naphthyridine, quinoxaline, cinnoline, carbazole, phenantridine, acridine, phenazine, thiazole, isothiazole, phenothiazine, oxazole, isooxazole, furazane, phenoxazine, 2-, 3- or 4-pyridyl, 2-, 3—, 4-, 5-, or 8-quinolyl, 1-, 3-, 4-, or 5-isoquinolinyl 1-, 2—, or 3-indolyl, and 2—, or 3-thienyl and the like. The group may be a terminal group or a bridging group.

The term “halogen” represents chlorine, fluorine, bromine or iodine. The term “halide” represents chloride, fluoride, bromide or iodide.

The term “optionally substituted,” when used to describe a chemical structure or moiety, refers to the chemical structure or moiety wherein one or more of its hydrogen atoms is optionally substituted with a chemical moiety or functional group such as alcohol, alkoxy, alkanoyloxy, alkoxycarbonyl, alkenyl, alkyl (e.g., methyl, ethyl, propyl, t-butyl), alkynyl, alkylcarbonyloxy (—OC(O)alkyl), amide (—C(O)NH-alkyl- or -alkylNHC(O)alkyl), amine (such as alkylamino, arylamino, arylalkylamino), aryl, aryloxy, azo, carbamoyl (—NHC(O)O-alkyl- or —OC(O)NH-alkyl), carbamyl (e.g., CONH₂, as well as CONH-alkyl, CONH-aryl, and CONH-arylalkyl), carboxyl, carboxylic acid, cyano, ester, ether (e.g., methoxy, ethoxy), halo, haloalkyl (e.g., —CCl₃, —CF₃, —C(CF₃)₃), heteroalkyl, isocyanate, isothiocyanate, nitrile, nitro, phosphodiester, sulfide, sulfonamido (e.g., SO₂NH₂), sulfone, sulfonyl (including alkylsulfonyl, arylsulfonyl and arylalkylsulfonyl), sulfoxide, thiol (e.g., sulfhydryl, thioether) or urea (—NHCONH-alkyl-).

The term “micro” as used herein is to be interpreted broadly to include dimensions from about 1 micron to about 1000 microns, about 1 micron to less than about 1000 microns, about 1 micron to about 900 microns, about 1 micron to about 800 microns, about 1 micron to about 700 microns, about 1 micron to about 600 microns, about 1 micron to about 500 microns, about 1 micron to about 400 microns, about 1 micron to about 300 microns, about 1 micron to about 200 microns, or from about 1 micron to about 100 microns.

The term “nano” as used herein is to be interpreted broadly to include dimensions in a nanoscale, i.e., less than about 1000 nm, about 1 nm to less than about 1000 nm, about 1 nm to about 900 nm, about 1 nm to about 800 nm, about 1 nm to about 700 nm, about 1 nm to about 600 nm, about 1 nm to about 500 nm, about 1 nm to about 400 nm, about 1 nm to about 300 nm, about 1 nm to about 200 nm, or from about 1 nm to about 100 nm. Accordingly, the term “nanostructures”, “nanoparticles”, “nanomaterials” and the like as used herein may include structures that have at least one dimension in the range of no more than said range. The term “nanostructures”, “nanoparticles”, “nanomaterials” and the like as used herein may include structures that have at least one dimension that is no more than about 100 nm, no more than about 90 nm, no more than about 80 nm, no more than about 70 nm, no more than about 60 nm, no more than about 50 nm, no more than about 40 nm, no more than about 30 nm, no more than about 20 nm, or no more than about 10 nm.

The term “nanostructure” as used herein broadly refers to an arrangement of interrelated elements in a system having at least one dimension in the nanoscale. The nanostructure described herein can include a nanoparticle, nanorod, nanofiber, nanoneedle, nanoplate, nanotube, and the like. The term “size” when used in the context of nanoparticle can refer to the diameter of the nanoparticle although it is not limited as such.

The term “particle” as used herein broadly refers to a discrete entity or a discrete body. The particle described herein can include an organic, an inorganic, a composite particle or a biological particle. The particle used described herein may also be a macro-particle that is formed by an aggregate of a plurality of sub-particles or a fragment of a small object. The particle of the present disclosure may be spherical, substantially spherical, or non-spherical, such as irregularly shaped particles or ellipsoidally shaped particles. The term “size” when used to refer to the particle broadly refers to the largest dimension of the particle. For example, when the particle is substantially spherical, the term “size” can refer to the diameter of the particle; or when the particle is substantially non-spherical, the term “size” can refer to the largest length of the particle.

The terms “coupled” or “connected” as used in this description are intended to cover both directly connected or connected through one or more intermediate means, unless otherwise stated.

The term “associated with”, used herein when referring to two elements refers to a broad relationship between the two elements. The relationship includes, but is not limited to a physical, a chemical or a biological relationship. For example, when element A is associated with element B, elements A and B may be directly or indirectly attached to each other or element A may contain element B or vice versa.

The term “adjacent” used herein when referring to two elements refers to one element being in close proximity to another element and may be but is not limited to the elements contacting each other or may further include the elements being separated by one or more further elements disposed therebetween.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

It will also be appreciated that where priority is claimed to an earlier application, the full contents of the earlier application is also taken to form part of the present disclosure and may serve as support for embodiments disclosed herein.

### DESCRIPTION OF EMBODIMENTS

Exemplary, non-limiting embodiments of a material comprising geminal metal atom pairs supported on a carrier, a catalyst comprising said material, a method of preparing said material, a method of catalyzing coupling reactions and related methods thereto are disclosed hereinafter.

### Material

There is provided a material comprising geminal metal atom pairs. In various embodiments, the geminal metal atom pairs are stabilized/supported on/anchored/attached/coordinated/linked/bonded/bound to a carrier/support within the material. In various embodiments, the geminal metal atom pairs are regularly or uniformly anchored/supported/distributed along/in/throughout the carrier/support. In various embodiments, the geminal metal atom pairs comprise single-atom sites paired in specific coordination and spatial proximity. Advantageously, such a unique structure/coordination/spatial proximity allows a plurality of geminal metal atom pairs to be incorporated into and supported on said carrier. In various embodiments therefore, the material comprises a high metal density. Even more advantageously, such a unique structure/coordination/spatial proximity imparts catalytic properties to the material, making the material ideal/attractive for use as a catalyst. In various embodiments, during catalysis, the plurality of geminal metal atom pairs act as adsorbents, advantageously enabling the adsorption of a wide range of reactants/substrates (having multiple coordination sites and/or sterically hindered/crowded/congested structures) over/on the geminal metal atom pairs, and therefore efficiently activating such reactants/substrates. In various embodiments, the plurality of geminal metal atom pairs are capable of adsorbing and/or activating the same or different types of reactants/substrates. For example, the material may demonstrate co-adsorption, with each geminal metal atom pair adsorbing two different reactants. In various embodiments, the material is capable of adsorbing two or more, three or more, or four or more different reactants/substrates, and subsequently activating said reactants/substrates.

In various embodiments, the material is substantially devoid of metal aggregates and/or clusters. Advantageously, the presence of geminal metal atom pairs avoids the risk of a restricted spatial environment and/or a limited capacity for adsorption of active reactants/substrates during catalysis, which may otherwise occur in the presence of metal aggregates and/or clusters.

In various embodiments, each geminal metal atom pair comprises two single/monomeric/mononuclear metal atoms (e.g. M¹ and M²). In various embodiments, the two single/monomeric/mononuclear metal atoms are termed as “geminal metal atom pair” and refers to the two single metal atoms being attached to the same carrier/support. In various embodiments, the two single metal atoms in each geminal metal atom pair are not attached/coordinated/linked/bonded (e.g., chemically or physically) to each other. That is, in various embodiments, the two single metal atoms are isolated/separated (e.g., electronically isolated/separated) from each other. It will be appreciated, however, that despite being electronically isolated, in various embodiments, the two single/monomeric/mononuclear metal atoms interact with each other via geminal interaction/coupling.

In various embodiments, each geminal metal atom pair is substantially regularly/uniformly/evenly spaced apart from each other. In various embodiments, the two single/monomeric/mononuclear metal atoms are substantially regularly/uniformly/evenly spaced apart from each other. In various embodiments, the average distance between each geminal metal atom pair (or between the two single/monomeric/mononuclear metal atoms) is from about 1.0 Å to about 10.0 Å, from about 1.5 Å to about 5.0 Å, no more than about 9.0 Å, no more than about 8.0 Å, no more than about 7.0 Å, no more than about 6.0 Å, no more than about 5.0 Å, no more than about 4.0 Å, no more than about 3.0 Å, no more than about 2.0 Å, or no more than about 1.5 Å. For example, the distance between each geminal metal atom pair (or between the two single/monomeric/mononuclear metal atoms/centres) may be about 5.00 Å, about 4.95 Å, about 4.90 Å, about 4.85 Å, about 4.80 Å, about 4.75 Å, about 4.70 Å, about 4.65 Å, about 4.60 Å, about 4.55 Å, about 4.50 Å, about 4.45 Å, about 4.40 Å, about 4.35 Å, about 4.30 Å, about 4.25 Å, about 4.20 Å, about 4.15 Å, about 4.10 Å, about 4.05 Å, about 4.04 Å, about 4.03 Å, about 4.02 Å, about 4.01 Å, about 4.00 Å, about 3.99 Å, about 3.98 Å, about 3.97 Å, about 3.96 Å, about 3.95 Å, about 3.90 Å, about 3.85 Å, about 3.80 Å, about 3.75 Å, about 3.70 Å, about 3.65 Å, about 3.60 Å, about 3.55 Å, about 3.50 Å, about 3.45 Å, about 3.40 Å, about 3.35 Å, about 3.30 Å, about 3.25 Å, about 3.20 Å, about 3.15 Å, about 3.10 Å, about 3.05 Å, or about 3.00 Å.

In various embodiments, the average M¹-M² or M¹ . . . M² distance is from about 1.0 Å to about 10.0 Å, from about 1.5 Å to about 5.0 Å, no more than about 9.0 Å, no more than about 8.0 Å, no more than about 7.0 Å, no more than about 6.0 Å, no more than about 5.0 Å, no more than about 4.0 Å, no more than about 3.0 Å, no more than about 2.0 Å, or no more than about 1.5 Å. For example, the average M¹-M² or M¹M² distance may be about 5.00 Å, about 4.95 Å, about 4.90 Å, about 4.85 Å, about 4.80 Å, about 4.75 Å, about 4.70 Å, about 4.65 Å, about 4.60 Å, about 4.55 Å, about 4.50 Å, about 4.45 Å, about 4.40 Å, about 4.35 Å, about 4.30 Å, about 4.25 Å, about 4.20 Å, about 4.15 Å, about 4.10 Å, about 4.05 Å, about 4.04 Å, about 4.03 Å, about 4.02 Å, about 4.01 Å, about 4.00 Å, about 3.99 Å, about 3.98 Å, about 3.97 Å, about 3.96 Å, about 3.95 Å, about 3.90 Å, about 3.85 Å, about 3.80 Å, about 3.75 Å, about 3.70 Å, about 3.65 Å, about 3.60 Å, about 3.55 Å, about 3.50 Å, about 3.45 Å, about 3.40 Å, about 3.35 Å, about 3.30 Å, about 3.25 Å, about 3.20 Å, about 3.15 Å, about 3.10 Å, about 3.05 Å, or about 3.00 Å. In various embodiments, the two single metal atoms/centres are regularly/uniformly/evenly spaced apart, with a ground-state separation of from about 0.10 nm to about 1.0 nm, from about 0.11 nm to about 0.9 nm, from about 0.12 nm to about 0.8 nm, from about 0.13 nm to about 0.7 nm, from about 0.14 nm to about 0.6 nm, from about 0.15 nm to about 0.5 nm, about 0.49 nm, about 0.48 nm, about 0.47 nm, about 0.46 nm, about 0.45 nm, about 0.44 nm, about 0.43 nm, about 0.42 nm, about 0.41 nm, about 0.40 nm, about 0.39 nm, about 0.38 nm, about 0.37 nm, about 0.36 nm, about 0.35 nm, about 0.34 nm, about 0.33 nm, about 0.32 nm, about 0.31 nm, or about 0.30 nm from each other. It will be appreciated that, in various embodiments, the distance between the two single metal atoms/centres is dependent on the carrier (e.g., position of the metal coordination sites in the carrier). Advantageously, the regular/uniform ground-state separation imparts coordination dynamics (i.e. dynamic/adaptive metal-metal coordination/coupling e.g., bridge-coupling of the geminal metal atoms/centres) to the material, thereby enabling site cooperativity.

In various embodiments, the carrier/support comprises one or more atom(s), functional group(s), part(s), and/or metal coordination site(s). In various embodiments, the atom(s), functional group(s), part(s), and/or metal coordination site(s) are metal free and/or substantially devoid of metal. In various embodiments, the carrier comprises a plurality of atom(s), functional group(s), part(s), and/or metal coordination site(s) that are periodically and/or abundantly present in/throughout the carrier. The plurality of atom(s), functional group(s), part(s), and/or metal coordination site(s) may be regularly/uniformly/evenly spaced apart from each other in/throughout the carrier. Advantageously, in various embodiments, the plurality of atom(s), functional group(s), part(s), and/or metal coordination site(s) in the carrier are designed to receive, coordinate, support and/or anchor the geminal metal atom pairs in a regular/ordered arrangement. In various embodiments therefore, the dynamic/adaptive coordination/coupling of the geminal metal atoms (imparted by the regular/uniform ground-state separation) is attributed to the regular/ordered arrangement of the metal coordination sites in the carrier. Advantageously, in various embodiments, the regular structure of the polymeric carrier (e.g., polymeric carbon nitride) allows/enables the formation of metal geminal sites, which are maximized at high metal densities, via a simple ion exchange and activation process.

In various embodiments, the two single metal atoms are each stabilized/supported on/attached/coordinated/linked/bonded/bound to different metal coordination site(s) of the carrier. That is, in various embodiments, each single metal atom is designed to bridge and/or link two different metal coordination site(s) together in the carrier. In various embodiments, each single metal atom is stabilized/supported/coordinated/linked/bonded/bound to two different metal coordination site(s) of the carrier in a diagonal or linear arrangement.

In various embodiments, the material comprises a structure represented by general formula (1); and a structure represented by general formula (2):

-A¹-M¹-A²- (1)

-A³-M²-A⁴- (2)
wherein
M¹ and M² are each independently a single/monomeric/mononuclear metal atom;
M¹ and M² is a pair of geminal metal atoms; and
A¹, A², A³ and A⁴ are each independently an atom, functional group, part or metal coordination site of the carrier/support.

A¹, A², A³ and A⁴ may be independently selected from the group consisting of —C—, —N—, —P— and —S—. In various embodiments, A¹, A², A³ and A⁴ are each independently —N—. For example, the material may comprise a —N-M¹-N-configuration and/or —N-M²-N— configuration. In various embodiments, the material comprises a diagonally coordinated a —N-M¹-N— configuration and/or a diagonally coordinated —N-M²-N— configuration.

In various embodiments, the carrier is metal-free or substantially devoid of metal atom/particle/ion. The carrier may comprise a nanocrystalline structure such as a polycrystalline having at least one dimension that is no more than about 100 nm, no more than about 90 nm, no more than about 80 nm, no more than about 70 nm, no more than about 60 nm, no more than about 50 nm, no more than about 40 nm, no more than about 30 nm, no more than about 20 nm, or no more than about 10 nm.

In various embodiments, the carrier comprises a polymer. In various embodiments therefore, the carrier is a polymeric carrier. In various embodiments, the polymeric carrier comprises a polymer (e.g., organic polymer) having one or more atoms selected from carbon, nitrogen, phosphorus, sulfur/sulphur, hydrogen, or the like, or combinations thereof. In various embodiments, the polymeric carrier is a carbon-containing/based, nitrogen-containing/based, phosphorus-containing/based and/or sulfur/sulphur-containing/based organic polymer. For example, the polymeric carrier may be a C—C containing/based, C—N containing/based, C—P containing/based and/or C—S containing/based organic polymer. Advantageously, in various embodiments, the use of a nanocrystalline polymeric carrier (e.g., polymeric carbon nitride) allows precise control over the proximity of metal centres.

In various embodiments, the polymeric carrier comprises one or more layer/sheet(s), or is derived therefrom. In various embodiments, the polymeric carrier is in the form of a layered structure. That is, in various embodiments, the polymeric carrier is designed to comprise polymeric chains of fused nitrogen-containing heterocycles that are assembled to form layers/sheets. The fused nitrogen-containing heterocycles may be fused triazine rings (e.g., 1,3,5-triazine rings). In various embodiments, the carrier comprises polymeric chains of two or more fused triazine rings, for example, the carrier/support may comprise polymeric chains having two fused triazine rings, polymeric chains having three fused triazine rings (e.g., heptazine), polymeric chains having four fused triazine rings, or polymeric chains having five fused triazine rings. Accordingly, in various embodiments, the polymeric carrier comprises a nitrogen rich polymer (e.g., carbon nitride polymer or polymeric carbon nitride (PCN)) or derivatives thereof. Advantageously, the layers/sheets of the polymeric chains which are bonded to each other or held together by van der Waals forces help imparts strength and stability to the polymeric carrier, allowing the carrier to provide/serve as a support for the material. Even more advantageously, the overall two-dimensional (2D) organic-metal lattice structure (e.g., 2D nanosheets) of the polymeric carrier provides a higher/larger surface area and reduced diffusion paths for charges to the material, therefore enhancing the catalytic performance/property of the material.

In various embodiments, the material is amenable to exfoliation or capable of being exfoliated into atomically thin nanostructures. The material may be provided in the form of an exfoliated nanostructure.

In various embodiments, the material has a nanostructure comprising one or more of the following shapes/structures: nanoflakes, nanosheets, nanolayers, nanoplates, nanorods, nanofibers, nanoneedles and nanotubes.

In various embodiments, the total number of metal atoms present in the material is an even number. For example, the total number of metal atoms present in the material may be at least about 2, at least about 4, at least about 6, at least about 8, at least about 10, at least about 12, at least about 14, at least about 16, at least about 18, at least about 20, at least about 22, at least about 24, at least about 26, at least about 28, at least about 30, at least about 32, at least about 34, at least about 36, at least about 38, at least about 40, at least about 42, at least about 44, at least about 46, at least about 48, at least about 50, at least about 60, at least about 70, at least about 80, at least about 90, at least about 100, at least about 150, at least about 200, at least about 250, at least about 300, at least about 350, at least about 400, at least about 450, at least about 500, at least about 550, at least about 600, at least about 650, at least about 700, at least about 750, at least about 800, at least about 850, at least about 900, at least about 950, at least about 1,000, at least about 2,000, at least about 3,000, at least about 4,000, at least about 5,000, at least about 6,000, at least about 7,000, at least about 8,000, at least about 9,000, or at least about 10,000.

In various embodiments, the material comprises a high metal density/concentration (due to the presence of plurality of geminal metal atom pairs). In various embodiments, the material comprises from about 0.1 wt % to about 50.0 wt %, from about 0.15 wt % to about 47.5 wt %, from about 0.2 wt % to about 45.0 wt %, from about 0.35 wt % to about 42.5 wt %, from about 0.5 wt % to about 40.0 wt %, from about 0.75 wt % to about 37.5 wt %, from about 1.0 wt % to about 35.0 wt %, from about 1.5 wt % to about 32.5 wt %, from about 2.0 wt % to about 30.0 wt %, from about 3.5 wt % to about 27.5 wt %, from about 5.0 wt % to about 25.0 wt %, from about 7.5 wt % to about 22.5 wt %, from about 10.0 wt % to about 20.0 wt %, from about 12.5 wt % to about 17.5 wt %, or about 15.0 wt % of metal.

In various embodiments, the metal comprises transition metal. For example, the metal atoms may be transition metal atoms selected from the group consisting of copper (Cu), zinc (Zn), nickel (Ni), cobalt (Co), iron (Fe), manganese (Mn), chromium (Cr), vanadium (V), titanium (Ti), scandium (Sc), yttrium (Y), zirconium (Zr), niobium (Nb), molybdenum (Mo), technetium (Tc), ruthenium (Ru), rhodium (Rh), palladium (Pd), silver (Ag), cadmium (Cd), lanthanum (La), hafnium (Hf), tantalum (Ta), tungsten (W), rhenium (Re), osmium (Os), iridium (Ir), platinum (Pt), gold (Au), mercury (Hg), actinium (Ac), and combinations thereof.

In various embodiments, the metal is low valent. That is, in various embodiments, the metal may have a low valence/valence of 1 or 2 or has a valence state/oxidation number/charge of +1 or +2. For example, the metal may be low valent (e.g., monovalent or divalent) metal such as Cu(I), Pd(I), Ag(I), Zn(II), Au(I), Ni(II), Co(II), Fe(II), Mn(II), Cr(II), V(II), Ti(II), Sc(II), Y(II), Zr(II), Nb(II), Mo(II), Tc(II), Ru(II), Rh(II), Cd(II), La(II), Hf(II), Ta(II), W(II), Re(II), Os(II), Ir(II), Pt(II), Hg(II) and Ac(II). Advantageously, the low valency (i.e. monovalence) of the metal centre activation enhances the catalytic performance/property of the material by lowering the activation barrier and promoting activation of reactants/substrates during catalysis.

In various embodiments, the material comprises a first/inner shell comprising the single/monomeric/mononuclear metal atom and a first atom of the carrier/support. In various embodiments, the interatomic distance between the single/monomeric/mononuclear metal atom and a first atom of the carrier/support in the first shell is from about 0.1 Å to about 5.0 Å, from about 0.2 Å to about 4.5 Å, from about 0.5 Å to about 4.0 Å, from about 1.0 Å to about 3.5 Å, from about 1.1 Å to about 3.0 Å, from about 1.2 Å to about 2.9 Å, from about 1.3 Å to about 2.8 Å, from about 1.4 Å to about 2.7 Å, from about 1.5 Å to about 2.6 Å, from about 1.6 Å to about 2.5 Å, from about 1.7 Å to about 2.4 Å, from about 1.8 Å to about 2.3 Å, from about 1.9 Å to about 2.2 Å, or from about 2.0 Å to about 2.1 Å.

In various embodiments, the material comprises a second/outer shell comprising the single/monomeric/mononuclear metal atom and a second atom of the carrier/support. In various embodiments, the interatomic distance between the single/monomeric/mononuclear metal atom and a second atom of the carrier/support in the second shell is from about 1.5 Å to about 5.0 Å, from about 1.6 Å to about 4.5 Å, from about 1.7 Å to about 4.0 Å, from about 1.8 Å to about 3.5 Å, from about 1.9 Å to about 3.0 Å, from about 2.0 Å to about 2.9 Å, from about 2.1 Å to about 2.8 Å, from about 2.2 Å to about 2.7 Å, from about 2.3 Å to about 2.6 Å, or from about 2.4 Å to about 2.5 Å.

In various embodiments, the material, geminal metal atoms and/or single metal atoms comprise catalytic active site(s) and/or are capable of serving as catalytic active site(s). In various embodiments, the active sites are different from existing technologies in the art. Embodiments of the material comprise two-metal-atom site (consisting of pairs of regularly separated low-valence single-atom metal sites), which is completely different from those of the art that involve a single-metal-atom site.

In various embodiments, the material is suitable for use as a catalyst or suitable for use in catalysis. In various embodiments, there is provided a library of heterogeneous catalyst materials comprising a plurality of pairs of geminal metal atoms, the geminal atoms are stabilized/supported on/coordinated/linked/bonded/bound to a carrier/support. The material may therefore also be termed as a “geminal-atom catalyst”, “solid-state catalyst”, “solid-state catalyst material” and/or “compound”.

In various embodiments, the catalyst comprises a heterogeneous catalyst. That is, in various embodiments, the catalyst exists in a phase that is different from that of the reaction mixture. For example, the catalyst may be in the form of a solid (or exists in solid phase) while the reaction mixture may be in the form of a liquid (or exists in liquid phase). Advantageously, use of the heterogeneous catalyst allows the recycling, reusing and/or recovering of the material without substantial change in its performance (e.g., efficiency) and/or leaching of metals into the environment. In various embodiments therefore, the material is substantially much safer, less or not toxic, greener, environmentally friendly/benign and/or has a reduced environmental footprint as compared to catalysts in the art that have a higher tendency of metal leaching. In various embodiments, the material is durable, stable and/or recyclable/reusable/recoverable. The material may be recycled/reused/recovered after at least 1, at least 2, at least 3, at least 4, at least 5, at least 6, at least 7, at least 8, at least 9, at least 10, at least 15, at least 20, at least 25 or at least 30 reaction cycles/uses. In various embodiments, the material is recyclable/reusable/recoverable after the above stated number times without a substantial loss in catalytic properties. In various embodiments, the material is recycled/reused/recovered by washing with aqueous medium (e.g., deionized water).

Advantageously, in various embodiments, the material is easily separated from the products after catalysis, which significantly lower the operation cost and ensure the quality of the products (e.g., as-obtained pharmaceuticals).

In various embodiments, the material, geminal metal atoms and/or single/monomeric/mononuclear metal atoms are capable of catalyzing coupling reactions with an activation barrier of about 0.25 eV, about 0.20 eV, about 0.19 eV, about 0.18 eV, about 0.17 eV about 0.16 eV, about 0.15 eV, or about 0.10 eV. Advantageously, in various embodiments, the activation barrier is no more than about 0.50 eV, no more than about 0.40 eV, no more than about 0.30 eV, no more than about 0.25 eV, no more than about 0.20 eV, no more than about 0.15 eV, or no more than about 0.10 eV.

In various embodiments, the material, geminal metal atoms and/or single metal atoms are capable of catalyzing coupling reactions. In various embodiments, the coupling reactions involve a wide range of reactants/substrates including but is not limited to heteroaryl halides (e.g., heteroaryl iodides, heteroaryl bromides, heteroaryl chlorides), aryl halides (e.g., aryl iodides, aryl bromides, aryl chlorides), reactants/substrates containing hydroxyl group(s), reactants/substrates containing amino group(s), reactants/substrates containing amide group(s), reactants/substrates containing sulfonamide group(s), reactants/substrates containing alkyne group(s), reactants/substrates containing thiol group(s), reactants/substrates containing electron rich/deficient aryl group(s), alcohols, halogenated alcohols (e.g., fluorinated alcohols), deuterated alcohols (e.g., deuterated methanol), heterocyclic compounds (e.g., imidazole, pyrazole, triazole), alkynes, thiols (e.g., alkylthiol/mercaptan or thiophenol), azides (e.g., alkylazide, arylazide), acetylenes (e.g., alkyl acetylene, aryl acetylene), L-menthol, DL-menthol, stigmasterol, DL-isoborneol, galactose, 1,2,3,4-diacetone galactose, testosterone, prednisolone, derivatives thereof, the like, and combinations thereof.

In various embodiments, the coupling reactions comprise cross-coupling reactions, cross-coupling bond formations, cycloadditions such as azide-alkyne cycloaddition, carbon-carbon bond formation (i.e. C—C coupling), carbon-heteroatom bond formation (e.g., C—N coupling, C—O coupling, C—S coupling etc), N-arylation, the like, and combinations thereof.

### Method of Catalyzing

There is provided a method of catalyzing coupling reactions, the method comprising:
(a-i) providing the material as disclosed herein; and
(a-ii) mixing said material with two or more reactants/substrates to be coupled under suitable conditions to obtain a coupled product.

In various embodiments, the method of catalyzing comprises heterogeneous catalysis. For example, the method of catalyzing comprises the use of a material (present in the form of a solid) placed in a reaction mixture (present in the form of a liquid).

In various embodiments, the step of providing the material comprises adding said material to an anhydrous solvent system under vacuum, in an inert atmosphere (e.g., in the presence of an inert gas such as argon or nitrogen), or in the absence of reactive gases such as oxygen (e.g., dissolved oxygen). Purging of inert gas maintains an oxygen-free system through the reaction process/duration/period and it will be appreciated that various inert gases may be used in embodiments of the method disclosed herein. In various embodiments, the anhydrous solvent system comprises anhydrous or dry solvent selected from the group consisting of formamide/methanamide, dimethylformamide (DMF), dimethylsulfoxide (DMSO), dioxane (e.g., 1,4-dioxane), acetonitrile, and the like, and combinations/mixtures thereof.

In various embodiments, the step of providing the material comprises adding said material to an aqueous solvent system under ambient conditions. For example, the material may be added to aqueous solvent system at ambient temperature and/or pressure. In various embodiments, the aqueous solvent system comprises aqueous solvent selected from the group consisting of pentanol, butanol, cyclohexanol, octanol, propanol, heptanol, hexanol, acetyl acetone, ethyl acetoacetate, benzyl alcohol, acetic acid, aminoethanol, ethanol, diethylene glycol, methanol, ethylene glycol, water, and the like and combinations/mixtures thereof.

In various embodiments, any solvent that effectively serves as a medium to contain the components of the reaction mixture (e.g., material, reactants/substrates) may be used in embodiments of the reaction mixture disclosed herein. In various embodiments, the solvent is capable of substantially dissolving the components present in the reaction mixture. The solvent may be selected from the group consisting of formamide/methanamide, dimethylformamide (DMF), dimethylsulfoxide (DMSO), dioxane (e.g., 1,4-dioxane), acetonitrile, pentanol, butanol, cyclohexanol, octanol, propanol, heptanol, hexanol, acetyl acetone, ethyl acetoacetate, benzyl alcohol, acetic acid, aminoethanol, ethanol, diethylene glycol, methanol, ethylene glycol, water, and the like, and mixtures/combinations thereof.

In various embodiments, the step of providing the material comprises adding said material to a base. In various embodiments, the base is an inorganic base or an organic base. The base may be an inorganic base selected from the group consisting of metal tert-butoxides, metal phosphates, metal carbonates, metal hydrogen carbonates, metal hydroxides, metal hydrides and mixtures thereof. For example, the base may be lithium tert-butoxide (LiOtBu), sodium tert-butoxide (NaOtBu), potassium tert-butoxide (KOtBu), lithium phosphate (Li₃PO₄), sodium phosphate (Na₃PO₄), tripotassium phosphate (K₃PO₄), lithium carbonate (Li₂CO₃), sodium carbonate (Na₂CO₃), potassium carbonate (K₂CO₃), caesium carbonate (Cs₂CO₃), sodium hydrogen carbonate (NaHCO₃), sodium hydroxide (NaOH), potassium hydroxide (KOH), caesium hydroxide (CsOH) and the like, and combinations/mixtures thereof.

In various embodiments, the step of providing the material comprises adding said material in an amount of from about 0.1 mol % to about 50.0 mol %, from about 0.2 mol % to about 45.0 mol %, from about 0.35 mol % to about 42.5 mol %, from about 0.5 mol % to about 40.0 mol %, from about 0.6 mol % to about 39.0 mol %, from about 0.7 mol % to about 38.0 mol %, from about 0.8 mol % to about 37.0 mol %, from about 0.9 mol % to about 36.0 mol %, from about 1.0 mol % to about 35.0 mol %, from about 1.2 mol % to about 34.0 mol %, from about 1.4 mol % to about 33.0 mol %, from about 1.6 mol % to about 32.0 mol %, from about 1.8 mol % to about 31.0 mol %, from about 2.0 mol % to about 30.0 mol %, from about 3.5 mol % to about 32.5 mol %, from about 5.0 mol % to about 25.0 mol %, from about 10.0 mol % to about 20.0 mol %, from about 11.0 mol % to about 19.0 mol %, from about 12.0 mol % to about 18.0 mol %, from about 13.0 mol % to about 17.0 mol %, about 14.0 mol %, about 15.0 mol %, or about 16.0 mol %.

In various embodiments, the coupling reactions involve a wide range of reactants/substrates including but is not limited to heteroaryl halides (e.g., heteroaryl iodides, heteroaryl bromides, heteroaryl chlorides), aryl halides (e.g., aryl iodides, aryl bromides, aryl chlorides), reactants/substrates containing hydroxyl group(s), reactants/substrates containing amino group(s), reactants/substrates containing amide group(s), reactants/substrates containing sulfonamide group(s), reactants/substrates containing alkyne group(s), reactants/substrates containing thiol group(s), reactants/substrates containing electron rich/deficient aryl group(s), alcohols, halogenated alcohols (e.g., fluorinated alcohols), deuterated alcohols (e.g., deuterated methanol), heterocyclic compounds (e.g., imidazole, pyrazole, triazole), alkynes, thiols (e.g., alkylthiol/mercaptan or thiophenol), azides (e.g., alkylazide, arylazide), acetylenes (e.g., alkyl acetylene, aryl acetylene), L-menthol, DL-menthol, stigmasterol, DL-isoborneol, galactose, 1,2,3,4-diacetone galactose, testosterone, prednisolone, derivatives thereof, the like, and combinations thereof.

In various embodiments, the coupling reactions comprise cross-coupling reactions, cross-coupling bond formations, cycloadditions such as azide-alkyne cycloaddition, carbon-carbon bond formation (i.e. C—C coupling), carbon-heteroatom bond formation (e.g., C—N coupling, C—O coupling, C—S coupling etc), N-arylation, the like, and combinations thereof.

In various embodiments, the coupling reaction comprises the following reaction:

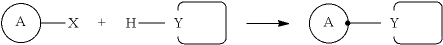

wherein
A is an optionally substituted monocyclic or polycyclic hydrocarbon ring system having 5 to 42 ring atoms, where up to three C atoms in each cyclic ring is/are optionally substituted with heteroatom(s) independently selected from the group consisting of O, N, S and NH, and where the ring system comprises 4-membered ring, 5-membered ring, 6-membered ring and/or mixtures thereof;
X is a halogen selected from the group consisting of F, Cl, Br and I;
Y is selected from the group consisting of O, N, C and S, and
wherein Y forms a part of an organic compound/nucleophile.

In various embodiments, A comprises an aromatic and/or heteroaromatic group selected from benzene, naphthalene, indene, anthracene, phenanthrene, fluorene, furan, thiophene, pyrrole, pyrazole, imidazole, oxazole, thiazole, triazole, oxadiazole, thiadiazole, tetrazole, benzodioxole (e.g, 1,3-benzodioxole), benzofuran, benzothiophene, benzopyrrole, benzodifuran, benzodithiophene, benzodipyrrole, dibenzofuran, dibenzothiophene, dibenzopyrrole/carbazole, pyridine, benzopyridine (e.g., isoquinoline and quinoline), bipyridine (e.g., 2,2′-bipyridine, 4,4′-bipyridine, 3,4′-bipyridine), pyridazine (1,2-diazine), bipyridazine (e.g., 3,3′-bipyridazine), pyrimidine (1,3-diazine), bipyrimidine (e.g., 2,2′-bipyrimidine), pyrazine (1,4-diazine), triazine (e.g., 1,2,3-triazine, 1,2,4-triazine and 1,3,5-triazine), phenanthroline, quinoxaline, phenazine, acridine or the like, or combinations thereof.

In various embodiments when Y is O, the organic compound/nucleophile containing Y is an alcohol. In various embodiments, the organic compound/nucleophile containing Y is halogenated alcohol (e.g., fluorinated alcohol). In various embodiments, the organic compound/nucleophile containing Y is deuterated alcohol (e.g., deuterated methanol).

In various embodiments when Y is N, the organic compound/nucleophile containing Y is a nitrogen-based nucleophile (e.g., nitrogen-containing heterocyclic compounds (e.g., imidazole, pyrazole, triazole), amine (e.g., aryl amine, alkyl amine), cyclic amine, amide, cyclic amide (or lactam), sulfonamide, cyclic sulfonamide (or sulfam)).

In various embodiments when Y is C, the organic compound/nucleophile containing Y is an alkyne (e.g., terminal alkyne). In various embodiments when Y is C, the organic compound/nucleophile containing Y is an electron-deficient (hetero)arene.

In various embodiments when Y is S, the organic compound/nucleophile containing Y is a thiol (e.g., alkylthiol/mercaptan or thiophenol).

In various embodiments, one or more hydrogen atoms in A and/or one or more hydrogen atoms in the organic compound/nucleophile containing Y is/are substituted with a chemical moiety or functional group such as alcohol, fluoroalcohol, alkoxy (e.g., methoxy, ethoxy), alkanoyloxy, alkoxycarbonyl, alkenyl, alkyl (e.g., methyl, ethyl, propyl, t-butyl), alkynyl, alkylcarbonyloxy (—OC(O)alkyl), amide (—C(O)NH-alkyl- or -alkylNHC(O)alkyl), amino/amine (such as alkylamino, arylamino, arylalkylamino), aryl, aryloxy, azo, carbamoyl (—NHC(O)O-alkyl- or —OC(O)NH-alkyl), carbamyl (e.g., CONH₂, CONH-alkyl, CONH-aryl, and CONH-arylalkyl), carboxyl, carboxylic acid, cyano, ester (e.g., acetate), ether (e.g., methoxy, ethoxy), halide, haloalkyl (e.g., —CCl₃, —CF₃, —C(CF₃)₃), heteroalkyl, isocyanate, isothiocyanate, nitrile, nitro, phosphodiester, sulfide, sulfonamido (e.g., SO₂NH₂), sulfone, sulfonyl (e.g. alkylsulfonyl, arylsulfonyl and arylalkylsulfonyl), sulfoxide, thiol (e.g., sulfhydryl, thioether), urea, or tosyl.

In various embodiments, the coupling reaction comprises the following reaction:

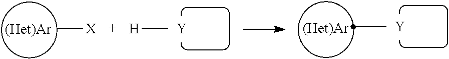

wherein
ring (Het)Ar comprises an optionally substituted aryl (i.e. Ar) and/or heteroaryl (i.e. HetAr).

In various embodiments, (Het)Ar comprises an optionally substituted aromatic and/or heteroaromatic group selected from benzene, naphthalene, indene, anthracene, phenanthrene, fluorene, furan, thiophene, pyrrole, pyrazole, imidazole, oxazole, thiazole, triazole, oxadiazole, thiadiazole, tetrazole, benzodioxole (e.g, 1,3-benzodioxole), benzofuran, benzothiophene, benzopyrrole, benzodifuran, benzodithiophene, benzodipyrrole, dibenzofuran, dibenzothiophene, dibenzopyrrole/carbazole, pyridine, benzopyridine (e.g., isoquinoline and quinoline), bipyridine (e.g., 2,2′-bipyridine, 4,4′-bipyridine, 3,4′-bipyridine), pyridazine (1,2-diazine), bipyridazine (e.g., 3,3′-bipyridazine), pyrimidine (1,3-diazine), bipyrimidine (e.g., 2,2′-bipyrimidine), pyrazine (1,4-diazine), triazine (e.g., 1,2,3-triazine, 1,2,4-triazine and 1,3,5-triazine), phenanthroline, quinoxaline, phenazine, acridine or the like, or combinations therefore.

In various embodiments, the method of catalyzing achieves a yield (of the coupled product) of at least about 50.0%, at least about 55.0%, at least about 60.0%, at least about 65.0%, at least about 70.0%, at least about 75.0%, at least about 80.0%, at least about 81.0%, at least about 82.0%, at least about 83.0%, at least about 84.0%, at least about 85.0%, at least about 86.0%, at least about 87.0%, at least about 88.0%, at least about 89.0%, at least about 90.0%, at least about 91.0%, at least about 92.0%, at least about 93.0%, at least about 94.0%, at least about 95.0%, at least about 96.0%, at least about 97.0%, at least about 98.0%, at least about 99.0%, at least about 99.1%, at least about 99.2%, at least about 99.3%, at least about 99.4%, at least about 99.5%, at least about 99.6%, at least about 99.7%, at least about 99.8%, or at least about 99.9%.

In various embodiments, the method of catalyzing achieves a conversion (of the coupled product) of at least about 50.0%, at least about 55.0%, at least about 60.0%, at least about 65.0%, at least about 70.0%, at least about 75.0%, at least about 80.0%, at least about 81.0%, at least about 82.0%, at least about 83.0%, at least about 84.0%, at least about 85.0%, at least about 86.0%, at least about 87.0%, at least about 88.0%, at least about 89.0%, at least about 90.0%, at least about 91.0%, at least about 92.0%, at least about 93.0%, at least about 94.0%, at least about 95.0%, at least about 96.0%, at least about 97.0%, at least about 98.0%, at least about 99.0%, at least about 99.1%, at least about 99.2%, at least about 99.3%, at least about 99.4%, at least about 99.5%, at least about 99.6%, at least about 99.7%, at least about 99.8%, or at least about 99.9%.

In various embodiments, the method of catalyzing achieves a selectivity or regioselectivity (of the coupled product) of at least about 50.0%, at least about 55.0%, at least about 60.0%, at least about 65.0%, at least about 70.0%, at least about 75.0%, at least about 80.0%, at least about 81.0%, at least about 82.0%, at least about 83.0%, at least about 84.0%, at least about 85.0%, at least about 86.0%, at least about 87.0%, at least about 88.0%, at least about 89.0%, at least about 90.0%, at least about 91.0%, at least about 92.0%, at least about 93.0%, at least about 94.0%, at least about 95.0%, at least about 96.0%, at least about 97.0%, at least about 98.0%, at least about 99.0%, at least about 99.1%, at least about 99.2%, at least about 99.3%, at least about 99.4%, at least about 99.5%, at least about 99.6%, at least about 99.7%, at least about 99.8%, or at least about 99.9%.

In various embodiments, the method of catalyzing achieves a turnover number (TON) of at least about 50, at least about 100, at least about 250, at least about 500, at least about 1,000, at least about 2,500, at least about 5,000, at least about 6,000, at least about 7,000, at least about 8,000, at least about 9,000, at least about 10,000, at least about 15,000, or at least about 20,000.

In various embodiments, the method comprises simultaneously catalyzing two or more different reactions.

In various embodiments, the two single/monomeric/mononuclear metal atoms (e.g. M¹ and M²) in the pair of geminal metal atoms are switchable from a first state where they are not coordinated/linked/bonded to each other (e.g. prior to catalysis or after catalysis when the product is desorbed/released), to a second state where they are coordinated/linked/bonded to each other (during catalysis).

In various embodiments, the method of catalyzing comprises catalyzing geminal-atom catalyzed organic cross-coupling reactions, which is different from other photocatalytic reactions such as photocatalytic CO₂ reduction, photocatalytic methane conversion, and/or photocatalytic H₂ evolution.

### Method of Preparing Material

In various embodiments, there is provided a method of preparing the material as disclosed herein, the method comprising:
(i) reacting a metal precursor/source with a carrier/support disclosed herein to obtain/precipitate the material; and
(ii) isolating the material.

In various embodiments, the reacting step is performed in the presence of a solvent. For example, the reacting step may comprise mixing the metal precursor/source with a carrier/support in the presence of a solvent to obtain a composition. Any solvent that effectively serves as a medium to contain the components of the composition (e.g., metal precursor/source, carrier/support) may be used in embodiments of the composition disclosed herein. In various embodiments, the solvent is capable of substantially dissolving the components present in the composition. The solvent may comprise a polar and/or water-soluble solvent. In various embodiments, the solvent is selected from the group consisting of formamide/methanamide, dimethylformamide (DMF), dimethylsulfoxide (DMSO), acetonitrile, pentanol, butanol, cyclohexanol, octanol, propanol, heptanol, hexanol, acetyl acetone, ethyl acetoacetate, benzyl alcohol, acetic acid, aminoethanol, ethanol, diethylene glycol, methanol, ethylene glycol, water and the like and combinations/mixtures thereof. It is to be appreciated that the type of solvent used is dependent on the type of metal precursor/source and carrier/support used, and is not limited to the above.

In various embodiments, the reacting step is performed at a temperature that is from about 20 °C to about 150 °C, from about 25 °C to about 145 °C, from about 30 °C to about 140 °C, from about 35 °C to about 135 °C, from about 40 °C to about 130 °C, from about 45 °C to about 125 °C, from about 50 °C to about 120 °C, from about 55 °C to about 115 °C, from about 60 °C to about 110 °C, from about 65 °C to about 105 °C, from about 70 °C to about 100 °C, from about 75 °C to about 95 °C, from about 80 °C to about 90 °C, or about 85 °C. For example, the reacting step may be performed at a room temperature that is from about 20 °C to about 40 °C, followed by performing at an elevated temperature that is from about 90 °C to about 150 °C.

In various embodiments, the reacting step is performed over a time duration of from about 5 minutes to about 48 hours, from about 10 minutes to about 36 hours, from about 15 minutes to about 24 hours, from about 20 minutes to about 12 hours, from about 25 minutes to about 11 hours, from about 30 minutes to about 10 hours, from about 1 hour to about 9 hours, from about 2 hours to about 8 hours, from about 3 hours to about 7 hours, from about 4 hours to about 6 hours, or about 5 hours.

In various embodiments, the reacting step comprises one or more of the following steps: dispersing, mixing, stirring, sonicating and/or ultrasonicating the mixture of metal precursor/source and carrier/support.

In various embodiments, the isolating step comprises one or more of the following steps: purifying, centrifuging, quenching and/or washing the material to remove impurities such as excess/free metal precursors and/or carrier/support. The step(s) of purifying, centrifuging, quenching and/or washing may be repeated at least 1 time, at least 2 times, at least 3 times, at least 4 times, at least 5 times, at least 6 times, at least 7 times, at least 8 times, at least 9 times, at least 10 times, at least 15 times, at least 20 times with a washing medium. The washing medium may be alcohol (e.g., ethanol, isopropyl alcohol).

In various embodiments, the method further comprises one or more of the following post reaction steps: drying and/or heating the material, optionally under vacuum or in an inert atmosphere. The step(s) of drying and/or heating may be performed in the presence of an inert gas such as argon or nitrogen. The step(s) of drying and/or heating may be performed at a temperature that is from about 50 °C to about 600 °C, from about 55 °C to about 550 °C, from about 60 °C to about 500 °C, from about 65 °C to about 450 °C, from about 75 °C to about 400 °C, from about 80 °C to about 350 °C, from about 85 °C to about 300 °C, from about 90 °C to about 250 °C, from about 95 °C to about 225 °C, from about 100 °C to about 200 °C, from about 110 °C to about 190 °C, from about 120 °C to about 180 °C, from about 130 °C to about 170 °C, from about 140 °C to about 160 °C, or about 150 °C. The step(s) of drying and/or heating may be performed over a time duration of from about 1 hour to about 24 hours, from about 2 hours to about 22 hours, from about 3 hours to about 20 hours, from about 4 hours to about 18 hours, from about 5 hours to about 16 hours, from about 6 hours to about 14 hours, from about 7 hours to about 12 hours, or from about 8 hours to about 10 hours or about 9 hours.

In various embodiments, the method further comprises a step of exfoliating the material and/or the carrier/support into atomically thin nanostructures. For example, the material may be exfoliated via sonication, mechanical, thermal, hydrothermal, electrochemical, laser-assisted, and/or microwave-assisted methods before use as a catalyst. For example, the carrier/support may be exfoliated via sonication, mechanical, thermal, hydrothermal, electrochemical, laser-assisted, and/or microwave-assisted methods before reacting with the metal precursor/source to form the material.

In various embodiments, the metal precursor/source comprises a salt. In various embodiments, the metal precursor/source/salt comprises metal chloride, metal fluoride, metal bromide, metal iodide, metal oxide, metal sulfate, metal sulfide, metal cyanide, metal thiocyanate, metal acetylide, metal acetate, metal hydroxide, metal hydride, metal nitrate, metal phosphide, metal phosphate, metal carbonate, metal carbonate hydroxide, metal chlorate, metal arsenate, metal azide, metal acetylacetonate, metal perchlorate, metal triflate, metal tetrafluoroborate, metal benzoate, metal chromite, metal peroxide, the like and combinations thereof.

In various embodiments, the metal precursor/source/salt comprises a divalent metal cation. In various embodiments, the metal precursor/source/salt comprises a copper salt selected from the group consisting of copper chloride (e.g., CuCl₂), copper fluoride (e.g., CuF₂), copper bromide (e.g., CuBr₂), copper iodide (e.g., CuI₂), copper oxide (e.g., CuO), copper sulfate (e.g., CuSO₄), copper sulfide (e.g., CuS), copper cyanide (e.g., CuCN₂), copper thiocyanate (e.g., Cu(SCN)₂), copper acetylide (e.g., Cu₂C₂), copper acetate (e.g., Cu(OAc)₂), copper hydroxide (e.g., Cu(OH)₂), copper hydride (e.g., CuH₂), copper nitrate (e.g., Cu(NO₃)₂), copper phosphide (e.g., CuP), copper phosphate (e.g., Cu₃(PO₄)₂), copper carbonate (e.g., CuCO₃), copper carbonate hydroxide (e.g., CuCO₃(OH)₂), copper chlorate (e.g., Cu(ClO₃)₂), copper arsenate (e.g., Cu₃(AsO₄)₂), copper azide (e.g., Cu(N₃)₂), copper acetylacetonate (e.g., Cu(O₂C₅H₇)₂), copper perchlorate (e.g., Cu(ClO₄)₂), copper triflate (e.g., Cu(OSO₂CF₃)₂), copper tetrafluoroborate (e.g., Cu(BF₄)₂), copper benzoate (e.g., Cu(C₆H₅CO₂)₂), copper chromite (e.g., Cu₂Cr₂O₅) and copper peroxide (e.g., CuO₂), the like, and combinations thereof.

In various embodiments, the reacting step comprises adding/incorporating from about 0.1 wt % to about 50.0 wt %, from about 0.2 wt % to about 45.0 wt %, from about 0.5 wt % to about 40.0 wt %, from about 1.0 wt % to about 35.0 wt %, from about 2.0 wt % to about 30.0 wt %, from about 5.0 wt % to about 25.0 wt %, from about 10.0 wt % to about 20.0 wt %, or about 15.0 wt % of the metal precursor/source.

In various embodiments, the reacting step comprises adding/incorporating from about 50.0 wt % to about 99.9 wt %, from about 55.0 wt % to about 99.8 wt %, from about 60.0 wt % to about 99.5 wt %, from about 65.0 wt % to about 99.0 wt %, from about 70.0 wt % to about 98.0 wt %, from about 75.0 wt % to about 95.0 wt %, from about 80.0 wt % to about 90.0 wt %, or about 85.0 wt % of the carrier/support.

In various embodiments, the material is formed/obtained via an ion exchange process where an ion/atom of the carrier/support is replaced with an ion/atom of the metal precursor. For example, the ion (attached to a metal coordination site of the carrier/support) may be replaced with a cation of the metal precursor.

In various embodiments, the material/catalyst is prepared by a post-synthetic method, whereby metals are introduced via an ion-exchange strategy which allows them to be anchored on the surface of the carrier (e.g., polymeric carbon nitride). Advantageously, embodiments of the presently disclosed method are unmatched by direct synthesis approaches of the art such as pyrolysis, wherein a large fraction of metals is embedded in the bulk of a structure and remains inaccessible.

In various embodiments, the material prepared from embodiments of the method disclosed herein enables a cooperative bridge-coupling pathway for a broad range of cross-coupling reactions with remarkable activity, selectivity, and stability, outperforming all so-far-known Cu nanocatalysts and even improving regioselectivity compared to the benchmarked homogeneous cuprous iodide catalysis. In various embodiments, the material prepared from embodiments of the method disclosed herein show much better catalysis activity and/or recyclability in a broad range of cross-coupling reactions as compared to commonly-used homogeneous catalytic systems (e.g., CuI). In various embodiments, the material prepared from embodiments of the method also show a wide substrate scope and/or long operation lifetime, stable recyclability, unmatched by homogeneous metal (e.g., Cu) catalysts.

Embodiments of the method are straightforward to perform, have a low production cost (i.e. cost effective) and may be carried out as a one-step direct synthesis method/one pot synthesis method of pharmaceutical compounds. Embodiments of the method may also be used in the synthesis of biorelevant compounds and translation to continuous flow synthesis to produce complex molecules. Advantageously, embodiments of the method are also environmentally benign, non-toxic and/or has substantially high scalability.

In various embodiments, the material prepared from embodiments of the method disclosed herein comprises one or more of the following characteristics or properties: broad applicability (e.g., work for most metal-catalyzed organic reactions), high (catalytic) performance in terms of yield (e.g., about 60-95%) and/or selectivity (about >90%), high catalytic activity, high and/or stable recyclability (e.g., more than about 10 times), high stability in continuous flow systems (e.g., stable drug synthesis in a continuous flow manner), low toxicity, low production/synthesis cost (e.g., about <S$200 per kilogram), wide substrate scope and long operation lifetime. In various embodiments, the material prepared from embodiments of the method disclosed herein is easily separated from the products after reaction, which significantly lowers the operation cost (e.g., about US$100/kg) and ensures the quality of as-obtained pharmaceuticals.

In various embodiments, the material prepared from embodiments of the method disclosed herein exhibits much higher stability and/or is readily translated to flow processes as compared to conventional homogeneous catalytic systems.

### BRIEF DESCRIPTION OF FIGURES

FIG. 1A is a schematic diagram 100 showing the strategy for the preparation of a material comprising a plurality of geminal metal atom pairs supported on a polymeric carrier (e.g., Cu_{g}/PCN geminal atom catalysts based on the periodic crystal structure of the host) in accordance with various embodiments disclosed herein. As shown in FIG. 1A, the carrier (e.g., polymeric carbon nitride (PCN) 110) comprises H atoms 102, C atoms 104, N atoms 106 and metal (e.g., Cu) ions/atoms 108. The presence of N—H functional group(s) 106-102 are present as metal coordination site(s) in the carrier. Upon introduction of metal (e.g., Cu) single atoms 108a, 108b, 108c and 108d, the Cu single atoms replace H atoms 102 via ion exchange process to coordinate/anchor/support themselves to the (N atoms 116 in the) carrier as geminal pairs, thereby obtaining Cu_{g}/PCN 112. As shown in FIG. 1A, Cu_{g}/PCN 112 comprises a plurality of geminal Cu pairs (e.g., see geminal Cu pair 118a and 118b; and geminal Cu pair 118d and 118c) coordinated/anchored/supported on the carrier. The geminal Cu pairs are stabilized by the N atoms 116.

FIG. 1B shows Fourier transform infrared (FTIR) spectra of PCN and Cu_{g}/PCN in accordance with various embodiments disclosed herein.

FIG. 1C shows an atomic-resolution ADF-STEM image of geminal Cu structures (see circled) in accordance with various embodiments disclosed herein. Scale bar represents 0.5 nm.

FIG. 1D shows k³ weighted Cu K-edge Fourier transformed extended X-ray absorption fine structure (EXAFS) spectra of Cu_{g}/PCN in accordance with various embodiments disclosed herein, and Cu foil as reference.

FIG. 1E shows an experimental high-resolution XANES spectrum compared with the calculated XANES data of optimized DFT-modelled structure of Cu_{g}/PCN in accordance with various embodiments disclosed herein.

FIG. 2A shows the substrate scope of Cu_{g}/PCN-catalyzed cross-couplings in accordance with various embodiments disclosed herein. Product yields obtained in Cu_{g}/PCN-catalyzed C—N bond formations of biorelevant molecules with a broad scope of (hetero)aryl halides are shown in the figure. Reaction conditions (1.4 mol % Cu): C—N bond formation, Aryl halide (0.2 mmol), nitrogen-based nucleophile (0.24 mmol for N-heterocyclic compounds, 0.3 mmol for primary and secondary amines), 0.4 mmol base (K₃PO₄ for N-heterocyclic compounds, NaOH for primary and secondary amines), anhydrous DMSO (1.0 mL), 110 °C, 28 h.

FIG. 2B shows the substrate scope of Cu_{g}/PCN-catalyzed cross-couplings in accordance with various embodiments disclosed herein. Product yields obtained in Cu_{g}/PCN-catalyzed C—O bond formations of biorelevant molecules with a broad scope of (hetero)aryl halides are shown in the figure. Reaction conditions (1.4 mol % Cu): C—O bond formation, Aryl iodide (0.2 mmol), alcohol (0.4 mmol), KOtBu (0.3 mmol), anhydrous dioxane (2.0 mL), 80 °C, 18 h.

FIG. 2C shows the substrate scope of Cu_{g}/PCN-catalyzed cross-couplings in accordance with various embodiments disclosed herein. Product yields obtained in Cu_{g}/PCN-catalyzed C—C and C—S bond formations of biorelevant molecules with a broad scope of (hetero)aryl halides are shown in the figure. Reaction conditions (1.4 mol % Cu): C—C bond formation, Aryl halide (0.2 mmol), alkyne (0.3 mmol, for di-halide aromatic ring, 0.6 mmol), Cs(OH)₂·H₂O (0.3 mmol, for di-halide aromatic ring, 0.6 mmol) and anhydrous DMSO (1.0 mL), 110 °C 28 h; C—S bond formation, Aryl halide (0.2 mmol), mercaptan/thiophenol (0.3 mmol), NaOtBu (0.30 mmol) and anhydrous dioxane (2.0 mL), 80 °C, 18 h.

FIG. 2D shows the substrate scope of Cu_{g}/PCN-catalyzed cross-couplings in accordance with various embodiments disclosed herein. Product yields obtained in Cu_{g}/PCN-catalyzed C—N, C—O, C—C, C—S bond formations and late-stage modification of biorelevant molecules with a broad scope of (hetero)aryl halides are shown in the figure.

FIG. 3A shows the proposed catalytic mechanism of C—O coupling over Cu_{g}/PCN in accordance with various embodiments disclosed herein. The figure shows a schematic illustration of the dynamic coordination of geminal Cu active centers.

FIG. 3B shows the proposed catalytic mechanism of C—O coupling over Cu_{g}/PCN in accordance with various embodiments disclosed herein. The figure shows the energy levels of Cu (II) . . . Cu (II) interaction with increasing proximity from distance (∞) to 2.46 Å.

FIG. 3C shows the proposed catalytic mechanism of C—O coupling over Cu_{g}/PCN in accordance with various embodiments disclosed herein. The figure shows the proposed reaction mechanism for C—O bond formation mediated by Cu_{g}/PCN. The spin state, valence state, and Cu . . . Cu distance are marked in the figure.

FIG. 3D shows the proposed catalytic mechanism of C—O coupling over Cu_{g}/PCN in accordance with various embodiments disclosed herein. The figure shows the calculated energy profiles for C—O bond formation mediated by Cu_{g}/PCN.

FIG. 4A shows the regioselectivity differences in the synthesis of 1,4-substituted imidazoles with different copper catalysts, including geminal-atom catalysis Cu_{g}/PCN in accordance with various embodiments disclosed herein.

FIG. 4B shows the C—N cross-coupling reaction catalyzed by Cu_{g}/PCN in accordance with various embodiments disclosed herein, in the presence of an equivalent of terpyridine.

FIG. 4C shows the comparison between a previous route and the Cu_{g}/PCN approach in accordance with various embodiments disclosed herein, in the synthesis of Dutasteride.

FIG. 4D shows the comparison between a previous Chan-Lam route and the newly designed Ullmann approach in accordance with various embodiments disclosed herein, in the synthesis of a xanthine precursor.

FIG. 4E shows the comparison between a previous route and the Cu_{g}/PCN approach in accordance with various embodiments disclosed herein, in the synthesis of a chiral spiro Cp ligand precursor.

FIG. 4F shows the continuous flow synthesis of 1-(4-methylbenzyl)-4-phenyl-1H-1,2,3-triazole as a function of time-on-stream over Cu_{g}/PCN in accordance with various embodiments disclosed herein and Cu tube, respectively. The inset shows the measured concentration of Cu ions leached into the solution at the end of each reaction.

FIG. 4G shows the life cycle assessment (LCA) impact assessment of heterogeneously catalyzed C—N coupling (top row) and homogeneously catalyzed C—N coupling (bottom row). Impact contributions are based on the performance of Cu_{g}/PCN in accordance with various embodiments disclosed herein or Cu₂O+L₁ (L₁=4,7-Dimethoxy-1,10-phenanthroline) in the synthesis of 1-(naphthalen-1-yl)-1H-imidazole through the coupling of imidazole and 1-iodonaphthalene. For heterogeneous catalyzed C—N coupling, global warming potential contributions are 38.6% 1-iodonaphthalene; 48.7% imidazole; 7.2% Cu_{g}/PCN; 5.6% DMSO; ecosystems quality contributions are 47.9% 1-iodonaphthalene; 38.7% imidazole; 7.4% Cu_{g}/PCN; 6.0% DMSO; human health contributions are 53.0% 1-iodonaphthalene; 31.8% imidazole; 9.3% Cu_{g}/PCN; 5.9% DMSO; and natural resources contributions are 27.5% 1-iodonaphthalene; 58.9% imidazole; 3.2% Cu_{g}/PCN; 10.4% DMSO. For homogeneous catalyzed C—N coupling, global warming potential contributions are 2.9% 1-iodonaphthalene; 3.6% imidazole; 77.1% Cu₂O/L₁; 16.5% N-methyl-2-pyrrolidone; ecosystems quality contributions are 4.1% 1-iodonaphthalene; 3.8% imidazole; 72.7% Cu₂O/L₁; 18.8% N-methyl-2-pyrrolidone; human health contributions are 5.5% 1-iodonaphthalene; 3.3% imidazole; 73.8% Cu₂O/L₁; 17.4% N-methyl-2-pyrrolidone; and natural resources contributions are 2.3% 1-iodonaphthalene; 5.0% imidazole; 75.3% Cu₂O/L₁; 17.4% N-methyl-2-pyrrolidone.

FIG. 5 shows scanning tunneling microscope (STM) characterization of Cu_{g}/PCN in accordance with various embodiments disclosed herein. The figure on the left shows an overview STM image after annealing PCN on Cu(111) at 370 K, V=2.0 V, I=20 pA. Scale bar represents 10 nm. The figure on the right shows the corresponding constant-height STM image with a CO-functionalized tip (V=5 mV, Δz=−20 pm; set point prior to turn off feedback, V=5 mV, I=200 pA. Scale bar represents 0.5 nm.

FIG. 6A shows characterization of Cu_{g}/PCN in accordance with various embodiments disclosed herein. This figure shows a high-resolution TEM image with the corresponding Fourier transform diffraction pattern inset. Scale bar represents 5 nm.

FIG. 6B shows characterization of Cu_{g}/PCN in accordance with various embodiments disclosed herein. This figure shows an ADF-STEM image and corresponding elemental maps. Scale bar represents 100 nm.

FIG. 6C shows characterization of Cu_{g}/PCN in accordance with various embodiments disclosed herein. This figure shows an AFM image of Cu_{g}/PCN with height profile inset.

FIG. 6D shows characterization of Cu_{g}/PCN in accordance with various embodiments disclosed herein. This figure shows XRD patterns of PCN and Cu_{g}/PCN.

FIG. 6E shows characterization of Cu_{g}/PCN in accordance with various embodiments disclosed herein. This figure shows Cu 2p XPS spectra of Cu_{g}/PCN. The peaks at 932.8 eV and 952.7 eV are assigned to Cu (I). The solid line and dot line represent the experiment and fitting curves, respectively.

FIG. 6F shows characterization of Cu_{g}/PCN in accordance with various embodiments disclosed herein. This figure shows EPR spectra of Cu_{g}/PCN and the reference CuCl₂.

FIG. 7A shows a comparison of the experimental and modelled Cu K-edge XANES spectra. The corresponding DFT-modelled atomic structures are shown in the inset.

FIG. 7B shows a comparison of the experimental and modelled Cu K-edge XANES spectra. The corresponding DFT-modelled atomic structures are shown in the inset.

FIG. 7C shows a comparison of the experimental and modelled Cu K-edge XANES spectra. The corresponding DFT-modelled atomic structures are shown in the inset.

FIG. 7D shows a comparison of the experimental and modelled Cu K-edge XANES spectra. The corresponding DFT-modelled atomic structures are shown in the inset.

FIG. 7E shows a comparison of the experimental and modelled Cu K-edge XANES spectra. The corresponding DFT-modelled atomic structures are shown in the inset.

FIG. 7F shows a comparison of the experimental and modelled Cu K-edge XANES spectra. The corresponding DFT-modelled atomic structures are shown in the inset.

FIG. 7G shows a comparison of the experimental and modelled Cu K-edge XANES spectra. The corresponding DFT-modelled atomic structures are shown in the inset.

FIG. 8A shows the substrate scope of Cu_{g}/PCN-catalyzed cross-coupling and cycloaddition in accordance with various embodiments disclosed herein. The figure shows product isolated yields obtained in Cu_{g}/PCN-catalyzed C—N bond formations (52-72).

FIG. 8B shows the substrate scope of Cu_{g}/PCN-catalyzed cross-coupling and cycloaddition in accordance with various embodiments disclosed herein. The figure shows product isolated yields obtained in Cu_{g}/PCN-catalyzed C—O bond formations (73-82). Products yields shown for Cu_{g}/PCN-catalyzed azide-alkyne cycloadditions (83-90) determined by gas chromatography.

FIG. 9 shows Cu_{g}/PCN-catalyzed pharmaceutical and biorelevant molecules prepared in accordance with various embodiments disclosed herein. The figure shows synthesis of pharmaceutical compounds (91-93) in multi steps and one-pot manner, and synthesis of bio-relevant molecules (94-96) from natural products.

FIG. 10A shows characterization of Cu₁/PCN with different Cu contents in accordance with various embodiments disclosed herein. This figure shows ADF-STEM images (scale bar: 1 nm) of Cu₁/PCN samples with different Cu contents. Dashed oval/elliptical shapes indicate GAC sites; and dashed circle/round shapes indicate SAC sites respectively.

FIG. 10B shows characterization of Cu₁/PCN with different Cu contents in accordance with various embodiments disclosed herein. This figure shows FTIR spectra of Cu₁/PCN samples with different Cu contents.

FIG. 10C shows characterization of Cu₁/PCN with different Cu contents in accordance with various embodiments disclosed herein. This figure shows Fourier-transformed EXAFS spectra of Cu₁/PCN samples with different Cu contents.

FIG. 11A shows electronic structure of Cu₁/PCN in accordance with various embodiments disclosed herein, with different Cu contents. This figure shows the Cu K-edge XANES.

FIG. 11B shows electronic structure of Cu₁/PCN in accordance with various embodiments disclosed herein, with different Cu contents. This figure shows the Cu 2p XPS spectra of Cu₁/PCN samples with different Cu contents.

FIG. 11C shows electronic structure of Cu₁/PCN 1100 in accordance with various embodiments disclosed herein, with different Cu contents. 1102 represents Cu; 1104 represents C; 1106 represents N; and 1108 represents H. This figure shows the charge density distributions and PDOS of monomeric Cu sites.

FIG. 11D shows electronic structure of Cu₁/PCN 1200 in accordance with various embodiments disclosed herein, with different Cu contents. This figure shows the charge density distributions and PDOS of geminal Cu sites. 1202 represents Cu; 1204 represents C; 1206 represents N; and 1208 represents H.

FIG. 12A shows in-situ Cu K-edge XANES and Fourier-transformed EXAFS spectra of Cu_{g}/PCN catalyst in accordance with various embodiments disclosed herein, measured before and in the C—O coupling reaction, respectively.

FIG. 12B shows in-situ Cu K-edge XANES spectra of Cu_{g}/PCN catalyst in accordance with various embodiments disclosed herein, measured before and in the C—O coupling reaction, respectively.

FIG. 12C shows in-situ EPR spectra of Cu_{g}/PCN in accordance with various embodiments disclosed herein, recorded at different times in the C—O coupling reaction.

FIG. 12A, FIG. 12B and FIG. 12C show in-situ XANES and EPR spectra during C—O coupling reaction. The chemical state change of Cu_{g}/PCN during the C—O cross-coupling reaction cycle was clearly seen from Cu K-edge XANES. A distinct weakening of the feature peak at 8983 eV and an increase of the main peak at 8996 eV indicate that the valence state of Cu increases during the reaction, which is related to the successful adsorption of 4-iodotoluene or methanol. Correspondingly, the intensity of the main peak related to the first coordination sphere in the Fourier transformed EXAFS spectra increases, evidencing the increased coordination number of Cu. The change in the chemical valence state of Cu during the reaction cycle is further elucidated by in-situ EPR spectroscopy. EPR-silent Cu (I) is first oxidized to EPR-sensitive Cu (II) with a gradually enhanced signal intensity and then reduced to the original Cu (I) state during a complete reaction process, in line with the in-situ XAFS results.

FIG. 13A shows stability of Cu_{g}/PCN in accordance with various embodiments disclosed herein, in C—O coupling. This figure shows cycling test for the coupling of 4-iodotoluene with ethanol to form C—O bond over Cu_{g}/PCN.

FIG. 13B shows stability of Cu_{g}/PCN in accordance with various embodiments disclosed herein, in C—O coupling. This figure shows FTIR spectra of the as-prepared Cu_{g}/PCN and the catalyst recovered after nine reaction cycles.

FIG. 13C shows stability of Cu_{g}/PCN in accordance with various embodiments disclosed herein, in C—O coupling. This figure shows XRD pattern of the as-prepared Cu_{g}/PCN and the catalyst recovered after nine reaction cycles.

FIG. 13D shows stability of Cu_{g}/PCN in accordance with various embodiments disclosed herein, in C—O coupling. This figure shows XPS spectra of the as-prepared Cu_{g}/PCN and the catalyst recovered after nine reaction cycles.

FIG. 13E shows stability of Cu_{g}/PCN in accordance with various embodiments disclosed herein, in C—O coupling. This figure shows Cu K-edge Fourier-transformed EXAFS of the as-prepared Cu_{g}/PCN and the catalyst recovered after nine reaction cycles.

FIG. 13F shows stability of Cu_{g}/PCN in accordance with various embodiments disclosed herein, in C—O coupling. This figure shows XANES spectra of the as-prepared Cu_{g}/PCN and the catalyst recovered after nine reaction cycles.

FIG. 14 shows the design principles of geminal-atom catalysis in accordance with various embodiments disclosed herein, illustrating how the present application overcome the current limitations or previously reported heterogeneous copper catalysts for organic synthesis to deliver high specific metal utilization and stability.

FIG. 15A shows the photos of three independent batches of Cu_{g}/PCN in accordance with various embodiments disclosed herein, prepared in gram scale. Experimental procedure for each batch of preparation conditions as shown in FIGS. 15A, 15B, 15C and 15D: CuCl₂ (3.2 g) and PCN (3.5 g) were dispersed in 1000 ml formamide, sonicated for 10 min then stirred in an oil bath (120 °C) for 12 h, following centrifugation and washing thoroughly by ethanol several times. The oven-dried powder (80 °C) was subsequently heated to 500 °C with a heating rate of 2 °C min⁻¹ and kept for 5 h with the protection of N₂ flow.

FIG. 15B shows the XRD patterns of three independent batches of Cu_{g}/PCN in accordance with various embodiments disclosed herein, prepared in gram scale. g-scale 1 refers to 18.4 wt % Cu, g-scale 2 refers to 18.1 wt % Cu, and g-scale 3 refers to 18.6 wt % Cu.

FIG. 15C shows the Cu K-edge XANES of three independent batches of Cu_{g}/PCN in accordance with various embodiments disclosed herein, prepared in gram scale. g-scale 1 refers to 18.4 wt % Cu, g-scale 2 refers to 18.1 wt % Cu, and g-scale 3 refers to 18.6 wt % Cu.

FIG. 15D shows the Fourier-transformed EXAFS spectra of three independent batches of Cu_{g}/PCN in accordance with various embodiments disclosed herein, prepared in gram scale. g-scale 1 refers to 18.4 wt % Cu, g-scale 2 refers to 18.1 wt % Cu, and g-scale 3 refers to 18.6 wt % Cu.

FIG. 16 shows reaction profile of Cu_{g}/PCN catalyzed C—O bonding formation in accordance with various embodiments disclosed herein. In a nitrogen-filled glovebox, 11 oven-dried screw-top reaction tubes were equipped with a magnetic stir bar. 4-lodotoluene (0.2 mmol×11, 479.7 mg), n-butanol (0.4 mmol×11, 326.1 mg), Potassium tert-butoxide (KOtBu, 0.3 mmol×11, 370.3 mg), decane (0.2 mmol×11, 313.0 mg) and anhydrous dioxane (2.0 mL×11) were sequentially added. Then shaking the mixture to make the homogeneous solution. Subsequently, Cu_{g}/PCN (4.0 mg) was weighed individually for every single tube. After that, the injector was used to transfer the aforementioned homogeneous solution to the 11 reaction tubes with 2 mL amount for each one. The reaction tubes were sealed with a Teflon-lined screw cap, removed from the glovebox, placed in an oil bath preheated to 80 °C for the indicated time. After cooling to rt, the reaction cap was removed. An aliquot of the solution was transferred into a GC vial and diluted with EtOAc. GC analysis was used for determination of the conversion and yield.

FIG. 17 shows Cu_{g}/PCN catalyzed p-ethoxytoluene synthesis in multi-gram scale in accordance with various embodiments disclosed herein. Experimental procedure for multi-gram synthesis of p-ethoxytoluene: In a nitrogen-filled glovebox, the aryl iodide (120 mmol), alcohol (240 mmol), Cu_{g}/PCN (600 mg, 1.4 mol % Cu), KOtBu (180 mmol) and anhydrous dioxane (1200 mL) were sequentially added to an oven-dried screw-top reaction bottle equipped with a stir bar. The reaction bottle was sealed with a Teflon-lined screw cap, removed from the glovebox, placed in an oil bath preheated to 80 °C and stirred for 18 h. After cooling to room temperature (rt), the cap was removed, and the reaction mixture was concentrated in vacuo with the aid of a rotary evaporator. The resulting residue was then purified by silica gel column chromatography to obtain the pure product.

FIG. 18 shows the strategy for preparing Cu₁/PCN (SACs) and Cu_{g}/PCN (GACs) based on the periodic crystal structure of the PCN host in accordance with various embodiments disclosed herein.

FIG. 19A shows characterization of ultra-high-density Cu₁/NC in accordance with various embodiments disclosed herein. This figure shows ADF-STEM image which evidences the high density of Cu single atoms. Scale bar represents 2 nm.

FIG. 19B shows characterization of ultra-high-density Cu₁/NC in accordance with various embodiments disclosed herein. This figure shows XRD patterns of NC and Cu₁/NC. Cu K-edge.

FIG. 19C shows characterization of ultra-high-density Cu₁/NC in accordance with various embodiments disclosed herein. This figure shows Cu K-edge XANES (the atomic structure is shown inset).

FIG. 19D shows characterization of ultra-high-density Cu₁/NC in accordance with various embodiments disclosed herein. This figure shows Fourier-transformed EXAFS spectra of Cu₁/NC.

FIG. 20A shows the geometric structure of the Cu_{g}/PCN 2000 in accordance with various embodiments disclosed herein. The figure shows the periodic structure optimized from VASP code. 2002 represents Cu; 2004 represents C; 2006 represents N; and 2008 represents H.

FIG. 20B shows the geometric structure of the Cu_{g}/PCN 3000 in accordance with various embodiments disclosed herein. The figure shows the cluster model used in the molecular DFT calculations. 3002 represents Cu; 3004 represents C; 3006 represents N; and 3008 represents H.

FIG. 21A shows indirect C—O coupling of CH₃O and C₆H₅ to form C₆H₅OCH₃ by periodic DFT modelling over Cu_{g}/PCN in accordance with various embodiments disclosed herein.

FIG. 21B shows indirect C—O coupling of CH₃O and C₆H₅ to form C₆H₅OCH₃ by molecular DFT modelling over Cu_{g}/PCN in accordance with various embodiments disclosed herein.

FIG. 21C shows the corresponding schematic structures of the intermediates (c) from the indirect C—O coupling of CH₃O and C₆H₅ to form C₆H₅OCH₃ over Cu_{g}/PCN in accordance with various embodiments disclosed herein. As shown in FIGS. 21A, 21B and 21C, as the Cu . . . Cu distance is almost 4 Å, direct C—O coupling is unlikely due to the large separation of *OCH₃ and *C₆H₅. Therefore, an indirect C—O coupling pathway was initially considered. In this pathway, the OCH₃ bonded with Cu_{B} will approach the near-by β-position carbon atom of *C₆H₅, and it will then migrate to the α-position carbon atom. Here α-position carbon atom denotes the carbon atom of *C₆H₅ directly bonded with Cu_{A}(II), and the β-position carbon atom is its nearest-neighbour in the benzene ring. It turns out that this indirect pathway is rather difficult because it involves a spin-forbidden reaction (³IS₄→¹IS₅) and a high barrier for ¹IS₅→¹TS₅ (0.98 eV).

FIG. 22 shows the radial distribution probability density (D(r)=r²R(r)²) of Cu (II) atomic orbitals (AOs).

FIG. 23 shows the calculated energy profiles for C—O bond formation catalyzed by isolated single-atom.

FIG. 24 shows the reaction energy profile for direct dehalogenation and dehydrogenation of C₆H₅I and CH₃OH into *C₆H₅+CH₃O*+HI without assistance of the base. The calculations show that the co-adsorption of C₆H₅I and CH₃OH on two Cu(I) atoms has an adsorption energy of −1.27 eV (singlet ¹IS₀→singlet ¹IS). However, the direct dehalogenation of C₆H₅I and dehydrogenation of CH₃OH to form HI and adsorbed *C₆H₅ and CH₃O* species on Cu(II) sites of the surface are strongly endothermic (2.52 eV), implying that it would have much higher reaction barrier. Note with direct bonding between C₆H₅ and CH₃O with copper, the Cu(I, d¹⁰) . . . Cu(I, d¹⁰) sites are converted into oxidized Cu(II, d⁹) . . . Cu(II, d⁹) sites. Therefore, the direct dehalogenation and dehydrogenation reaction from singlet ¹IS to triplet ³FS₀ is a spin-forbidden reaction, and no attempt is made to determine its reaction barrier. This result agrees with the experimental fact that the reaction cannot occur without addition of strong base.

FIG. 25 shows the reaction energy profile for direct dehydrogenation CH₃OH by KOtBu and subsequent dehalogenation of C₆H₅I to form Cu(II)*C₆H₅+CH₃O*Cu(II) over Cu_{g}/PCN, with releasing KI and HOtBu in solution. When a strong base like KO^{t}Bu is added to the system, it will immediately deprotonate CH₃OH to form K⁺⁻OCH₃ and HOtBu (singlet ¹IS₁→singlet ¹IS₂), with an exothermicity of −2.41 eV. This process is rather easy because of the higher pKa of HOtBu (17.0) compared to CH₃OH (15.5). The reaction between K⁺⁻OCH₃ and weakly adsorbed C₆H₅I to form the KI salt and adsorbed *C₆H₅ and CH₃O* species on Cu(II) sites of the surface will be difficult again, as it involves another spin-forbidden reaction to form C₆H₅—Cu(II, d⁹) and Cu(II, d⁹)—OCH₃. Although this process (singlet ¹IS₂→* triplet ³IS₃) is only slightly endothermic (−0.33 eV), the reaction rate of this spin-forbidden reaction will be very low because singlet-to-triplet spin crossing is necessary via a relatively small spin-orbit coupling, which makes this process the rate-determining step (RDS) of the whole reaction. Indeed, experimentally, EPR measurements identified the formation of Cu(II) during the reaction. In addition, the crucial role of strong base of KO^{t}Bu and the experimentally observed reaction time of C₆H₅I<C₆H₅Br<<C₆H₅Cl agrees with this computational result. It is worth mentioning that upon nucleation, the KI molecules will form KI solid deposit to drive the chemical equilibrium toward formation of triplet ³IS₃ product.

FIG. 26 shows EPR spectra of Cu_{g}/PCN measured under C-0 coupling reaction conditions.

FIG. 27 shows calculated PDOS of Cu sites in Cu_{g}/PCN bonding with aryl halide (4-iodobenzene) and alcohol (methanol), respectively. Briefly, comparison of the PDOS in Cu_{g}/PCN before and after (FIG. 27) reactant adsorption indicates that the interaction with the reactants shifts the PDOS of the copper centres to higher energies, crossing the Fermi energy (EF). This is because the copper atoms lose more electrons to the adsorbed reactants, as supported by the Bader charge analysis (Table 4). Furthermore, the adsorption changed the non-spin polarized electronic states of copper to spin-polarized with an average magnetic momentum of 1 μ_{B}/Cu.

FIG. 28A shows kinetic studies of Cu₁/PCN containing 18.3 wt. % (GAC) or 1.8 wt. % (SAC) Cu catalysed C—O bonding formation. The plots of the natural logarithm of the concentration of aryl halide versus time: initial concentration of aryl halide C₀=0.05 M (top left), C₀=0.1 M (top right and bottom). All reactions were conducted with 1.4 mol % Cu. Experimental procedure for FIGS. 28A, 28B and 28C: In an N₂-filled glovebox, 1.4 mol. % of Cu₁/PCN (corresponding to 1.0 mg for 18.3 wt. % Cu₁/PCN or 10 mg for 1.8 wt. % Cu₁/PCN) was added to each of five oven-dried screw-top reaction tubes equipped with stir bars. Subsequently, a premixed stock solution containing 0.1 mmol (C₀=0.05 M) or 0.2 mmol (C₀=0.1 M) of 4-iodotoluene, 0.4 mmol n-butanol, 0.3 mmol KOtBu, and 0.2 mmol decane (internal standard for GC analysis) in anhydrous dioxane (2.0 mL) was added to each reaction tube. The reaction tubes were sealed with Teflon-lined screw caps, removed from the glovebox, and placed in an oil bath preheated to 80 °C for the desired time (1-5 h). After cooling to rt, the reaction cap was removed. An aliquot of the solution was transferred into a vial, diluted with EtOAc, and the reactant conversion was analysed by gas chromatography. Under the optimized reaction conditions, the natural logarithm of the concentration of aryl halide versus time evidences a straight line, indicating that the reaction is first order in aryl halide. Consistently, the first-order rate constants (−slop, h⁻¹) at initial concentrations of 0.05 M or 0.1 M are very similar (0.431 h⁻¹ or 0.448 h⁻¹, respectively) on a 1.4 mol % Cu basis over the Cu_{g}/PCN catalyst with 18.3 wt. % metal content. Therefore, the aryl halide likely participates in the rate-limiting step. This finding agrees with the computational studies, which predict the activation of 4-iodobenzene (from IS2 to IS3, see FIG. 3C) to be rate-limiting. In contrast, the reaction using the 1.8 wt. % Cu₁/PCN catalyst proceeded significantly slower than the 18.3 wt. % Cu_{g}/PCN catalyst, despite the equivalent amount of copper introduced in the reaction (1.4 mol %). Comparatively, the first-order rate constant was 5.5 times higher over the material containing 18.3 wt. % copper, consistent with the higher yield observed over this catalyst.

FIG. 28B shows kinetic studies of Cu₁/PCN containing 18.3 wt. % (GAC) or 1.8 wt. % (SAC) Cu catalysed C—O bonding formation. The plots of the natural logarithm of the concentration of aryl halide versus time: initial concentration of aryl halide C₀=0.05 M (top left), C₀=0.1 M (top right and bottom). All reactions were conducted with 1.4 mol % Cu.

FIG. 28C shows kinetic studies of Cu₁/PCN containing 18.3 wt. % (GAC) or 1.8 wt. % (SAC) Cu catalysed C—O bonding formation. The plots of the natural logarithm of the concentration of aryl halide versus time: initial concentration of aryl halide C₀=0.05 M (top left), C₀=0.1 M (top right and bottom). All reactions were conducted with 1.4 mol % Cu.

FIG. 29A shows product analysis in Ullmann type coupling over Cu_{g}/PCN by NMR.

FIG. 29B shows product analysis in Ullmann type coupling over Cu_{g}/PCN by NMR.

FIG. 29C shows product analysis in Ullmann type coupling over Cu_{g}/PCN by NMR.

FIG. 30 shows ADF-STEM image of the used Cu_{g}/PCN catalyst in C—O coupling reaction. Scale bar represents 2 nm.

FIG. 31A shows cycling test for the coupling of 4-iodotoluene with ethanol to form C—O bond over Cu_{g}/PCN.

FIG. 31B shows cycling test for the coupling of 4-iodotoluene with ethanol to form C—O bond over Cu_{g}/PCN. This figure shows XRD pattern of the as-prepared Cu_{g}/PCN and the catalyst after four reaction cycles. For the first four cycles, the catalyst was washed with dioxane before use in the next cycle reaction. For reactivation, the catalyst was carefully cleaned with ethanol and water to remove KI before it was used in the fifth reaction cycle.

FIG. 31C shows cycling test for the coupling of 4-iodotoluene with ethanol to form C—O bond over Cu_{g}/PCN. This figure shows Cu K-edge XANES of the as-prepared Cu_{g}/PCN and the catalyst after four reaction cycles. For the first four cycles, the catalyst was washed with dioxane before use in the next cycle reaction. For reactivation, the catalyst was carefully cleaned with ethanol and water to remove KI before it was used in the fifth reaction cycle.

FIG. 31D shows cycling test for the coupling of 4-iodotoluene with ethanol to form C—O bond over Cu_{g}/PCN. This figure shows Fourier-transformed EXAFS spectra of the as-prepared Cu_{g}/PCN and the catalyst after four reaction cycles. For the first four cycles, the catalyst was washed with dioxane before use in the next cycle reaction. For reactivation, the catalyst was carefully cleaned with ethanol and water to remove KI before it was used in the fifth reaction cycle. As shown in FIGS. 31A, 31B, 31C and 31D, the deposited insoluble KI salts can be clearly seen from the XRD pattern, where a complete set of peaks belonging to KI crystals appeared in the used catalyst. At the same time, Cu K-edge Fourier-transformed EXAFS and XANES spectroscopy showed that the coordination and electronic structure of the copper atoms were identical before and after the reaction, meaning that the deposited KI salts were only physically covered on the surface of the catalyst, rather than chemically bound to the copper sites.

FIG. 32A shows the continuous flow setup used to evaluate the catalytic performance. This figure shows Cu_{g}/PCN catalyzed C—O bonding formation.

FIG. 32B shows the continuous flow setup used to evaluate the catalytic performance. This figure shows Cu_{g}/PCN catalyzed azide-alkyne cycloaddition.

FIG. 32C shows the continuous flow setup used to evaluate the catalytic performance. This figure shows Cu tube catalyzed azide-alkyne cycloaddition.

FIG. 33 shows the continuous flow synthesis of ethoxybenzene over Cu_{g}/PCN under various flow rates.

FIG. 34A shows sankey diagram of embodied GWP flows for homogeneously and heterogeneously catalyzed C—N coupling. Impact contributions are based on the performance of Cu_{g}/PCN (FIG. 2A) in the synthesis of product 1, expressed in kilogram of CO₂ equivalent per kilogram of product according to the IPCC 2021 definition. The analysis considers the optimized conditions reported for both systems. For simplicity, only contributions above a 3% cut-off are shown.

FIG. 34B shows sankey diagram of embodied GWP flows for homogeneously and heterogeneously catalyzed C—N coupling. Impact contributions are based on the performance of Cu₂O+L₁ (Table 2, Entry 2) in the synthesis of product 1, expressed in kilogram of CO₂ equivalent per kilogram of product according to the IPCC 2021 definition. The analysis considers the optimized conditions reported for both systems. For simplicity, only contributions above a 3% cut-off are shown.

FIG. 35 shows comparative impact derived from an ex-ante LCA of using a homogeneous or heterogeneous catalysts in one of the studied C—N coupling reactions based on four sustainability metrics: the midpoint global warming potential, ecosystems quality, endpoints human health, and resources. The base case considers a single use of the catalytic system. Reported values are per kg of product 1. DALY=daily adjusted life years.

FIG. 36A shows sensitivity analysis on solvent (a) recyclability of the reaction mixture for the homogeneous and heterogeneous catalytic systems in the C—N coupling reaction.

FIG. 36B shows sensitivity analysis on catalyst recyclability of the reaction mixture for the homogeneous and heterogeneous catalytic systems in the C—N coupling reaction. As shown in FIGS. 36A and 36B, the LCA calculations were repeated considering that both the catalyst (Cu_{g}/PCN or Cu₂O/L₁) and solvent could be re-used multiple times by recovering them from the reaction media without any energy penalty, assuming 1% loss of catalyst and solvent in each cycle and neglecting potential catalyst deactivation. Hence, the sensitivity analysis, which complements the evaluation of the two catalytic systems presented in the main manuscript, covers the performance of a wide range of more efficient homogenous and heterogeneous systems with different catalyst and solvent requirements. In the heterogeneous case, recycling does not significantly affect the total impact because this is given mostly by the reactants. In contrast, the carbon footprint of the homogenous system decreases with the recyclability level, first sharply and later with a milder slope as the bottleneck starts to shift to the reactants. The heterogeneous system would still outperform the homogenous system after re-using the catalyst and solvent 100 times under these ideal conditions. Hence, the results suggest that it would be highly unlikely to find a homogenous system outperforming the heterogeneous counterpart substantially.

### EXAMPLES

Example embodiments of the disclosure will be better understood and readily apparent to one of ordinary skill in the art from the following examples, tables and if applicable, in conjunction with the figures. It should be appreciated that other modifications related to structural, and/or chemical changes may be made without deviating from the scope of the invention. Example embodiments are not necessarily mutually exclusive as some may be combined with one or more embodiments to form new example embodiments. The example embodiments should not be construed as limiting the scope of the disclosure.

The following examples describe a new class of heterogeneous geminal-atom catalysts (GACs), which pair single-atom sites in specific coordination and spatial proximity. Regularly separated nitrogen-related anchoring groups with delocalized π-bond nature in a polymeric carbon nitride host permits the synthesis of Cu geminal sites with a ground-state separation of ~4 Å at high metal density. The adaptable coordination of individual Cu sites in GACs enables a cooperative bridge-coupling pathway via dynamic Cu—Cu bonding for diverse C—X (X=C, N, O, S) cross coupling with a low activation barrier. In the following examples, in-situ characterization and quantum-theoretical studies revealed that such a dynamic process is exclusively triggered by the adsorption of two different reactants over geminal metal sites, rendering homo-coupling unfeasible. These intrinsic advantages of GACs enable the efficient assembly of challenging heterocycles with multiple coordination sites, sterically congested scaffolds, and pharmaceuticals with highly specific and stable activity. Scale-up experiments and translation to continuous flow demonstrated its broad applicability for fine chemicals manufacture.

### Example 1: Heterogeneous Geminal-Atom Catalysts (GACs)

A new class of heterogeneous geminal-atom catalysts (GACs), consisting of pairs of regularly separated low-valence single-atom metal sites has been developed, which provides a powerful, economically-vital generalized platform to solve the longstanding reactivity challenge beyond conventional cross-coupling protocols, enabling us to implement environmentally benign organic synthesis by adjusting the nature and combination of the geminal metals to promote industrially-crucial coupling reactions.

The newly developed class of heterogeneous geminal-atom catalysts (GACs) comprises pairs of low-valence metal centers with a regular ground-state separation and suitable coordination dynamics to enable site cooperativity. In the following examples, the concept is demonstrated for copper atoms anchored on a nanocrystalline polymeric carbon nitride carrier, which defines the proximity of metal sites of ~4 Å and enables their adaptive coordination during the reaction. Catalytic evaluation in a broad range of cross-coupling reactions including azide-alkyne cycloaddition, carbon-carbon and carbon-heteroatom bond formation, demonstrates the superior performance of GACs compared to conventional SACs with a similar metal density based on a nitrogen-doped carbon host. The results show that GACs overcome a long-standing issue of sluggish oxidative addition in copper catalyzed cross-coupling reactions, which has limited the scope of copper compared to palladium despite its lower cost and carbon footprint. Detailed structural and mechanism analysis confirms the cooperativity of metal centers in the geminal sites, enabling the efficient activation of substrates through an unprecedented dynamic bridge-coupling mechanism. Further demonstration in the production of biorelevant pharmaceuticals and translation to continuous flow illustrate the broad synthetic capability of GACs compared to established heterogeneous catalysts. Quantification of the environmental benefits of the present GACs route compared to conventional homogeneous synthesis via an ex-ante lifecycle assessment (LCA) highlights the substantially reduced footprint in four well-established metrics.

### Example 2: Synthesis and Characterization of Geminal-Atom Catalysts

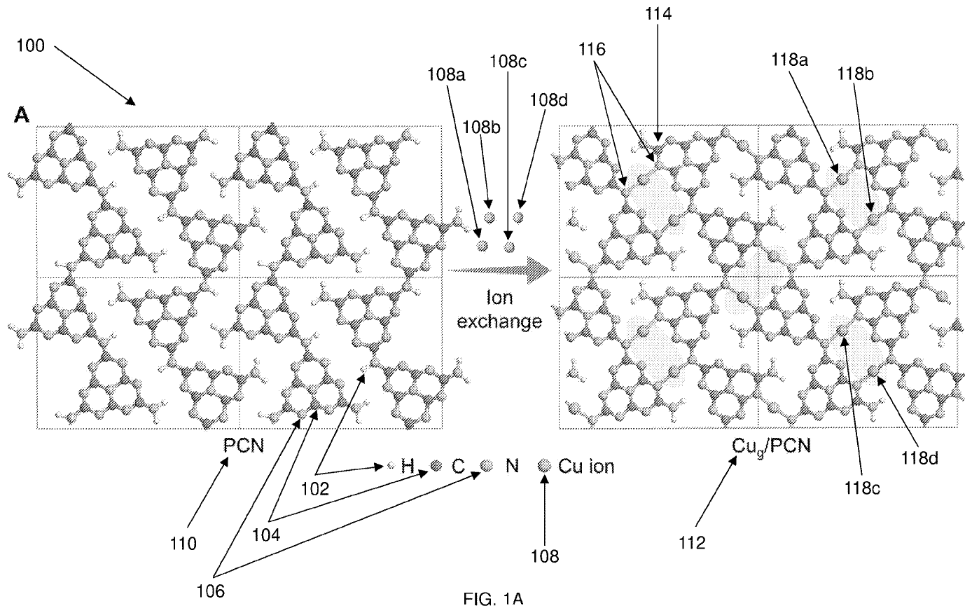

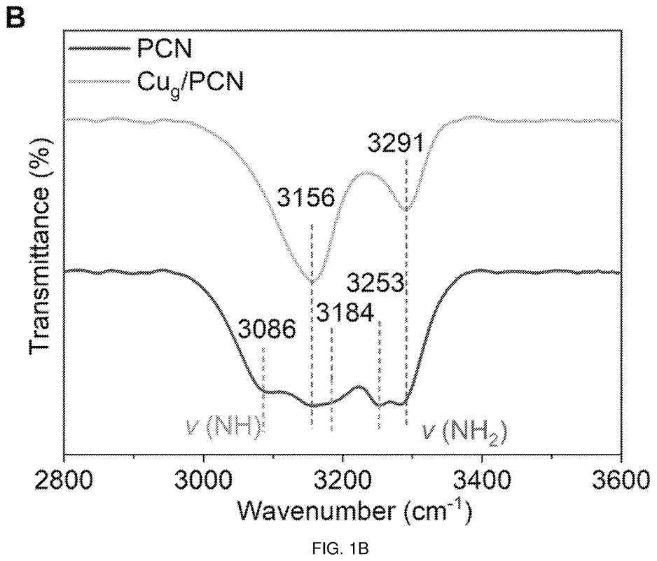

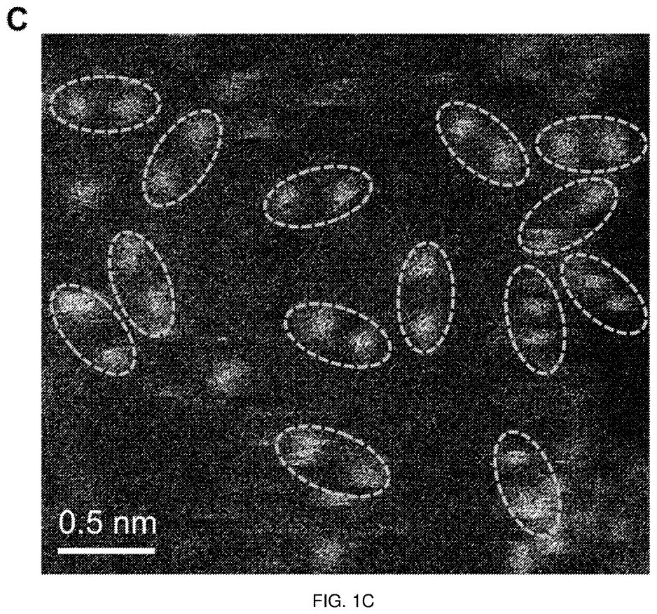

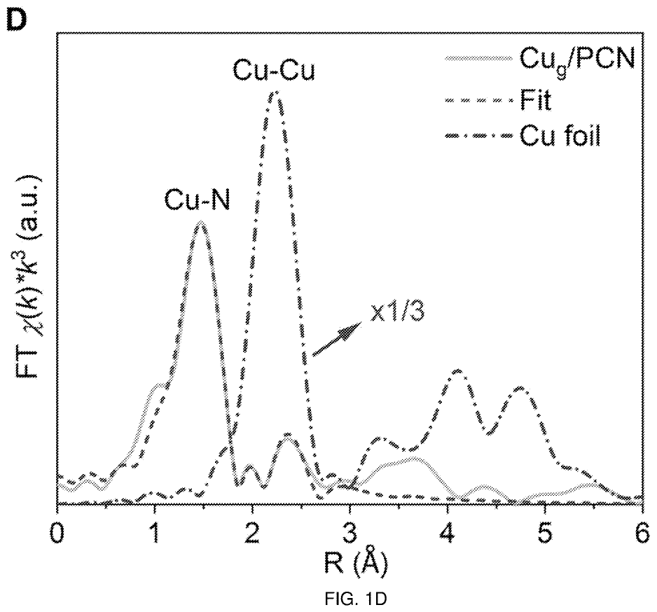

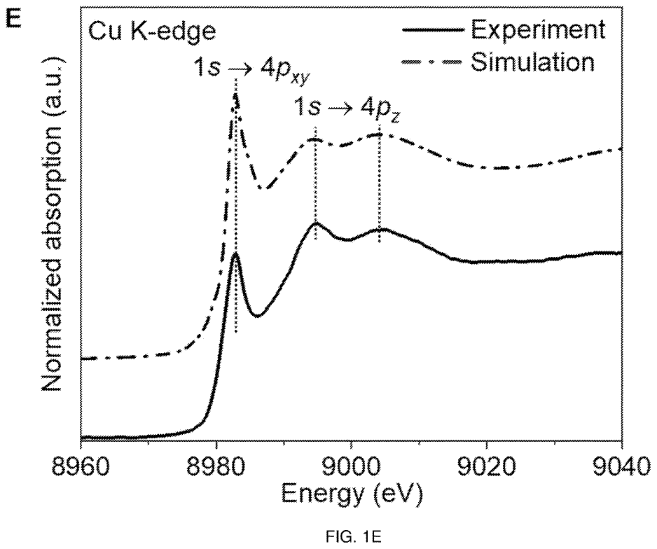

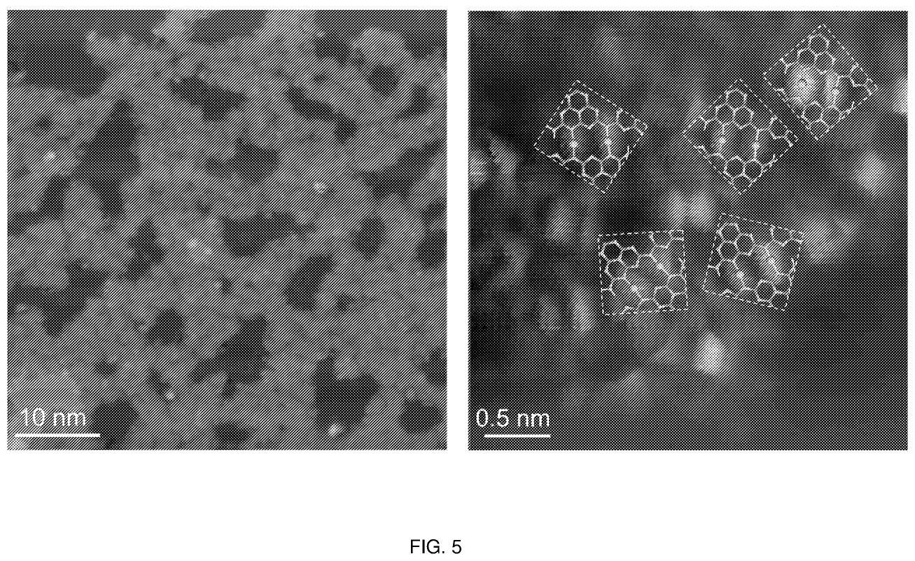

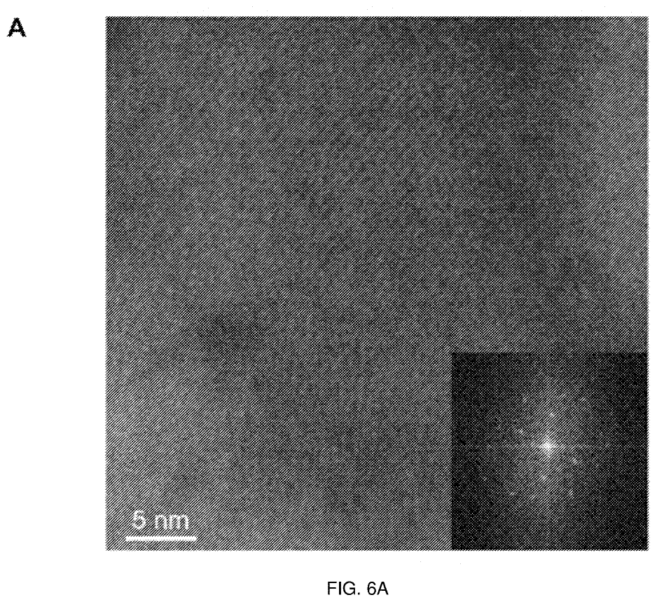

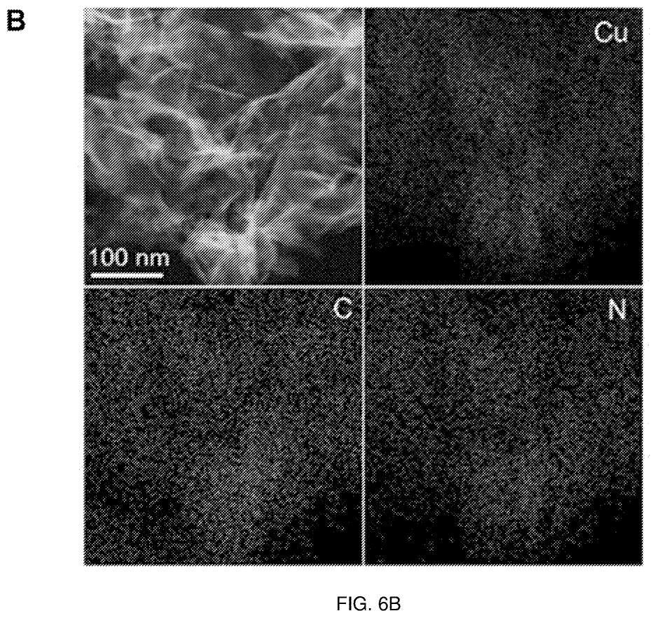

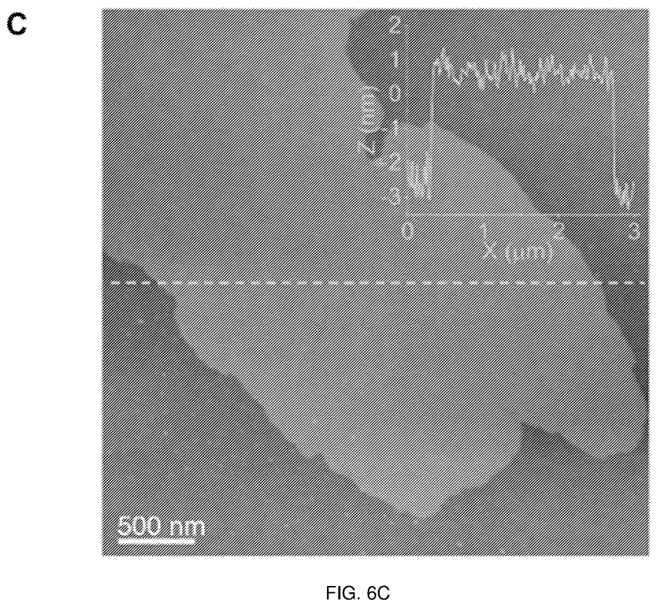

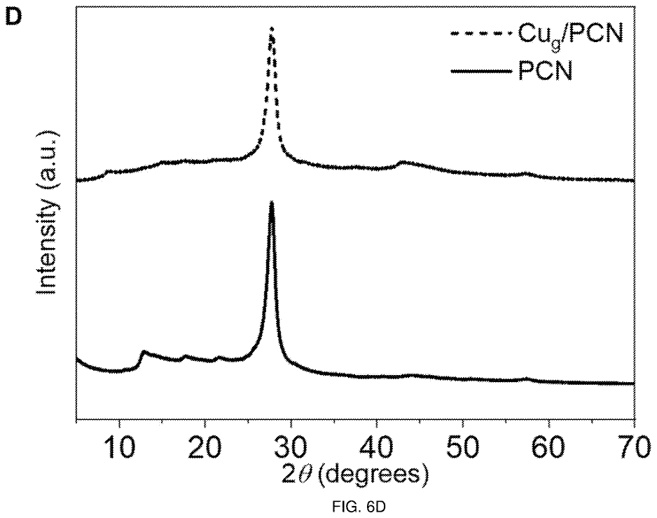

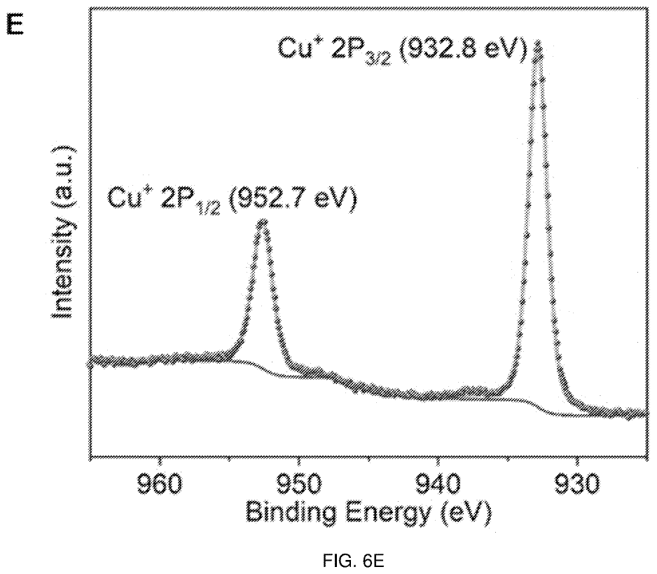

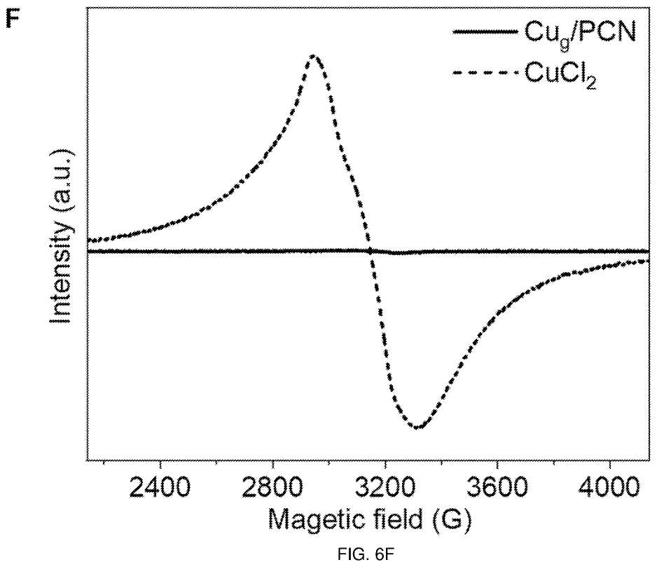

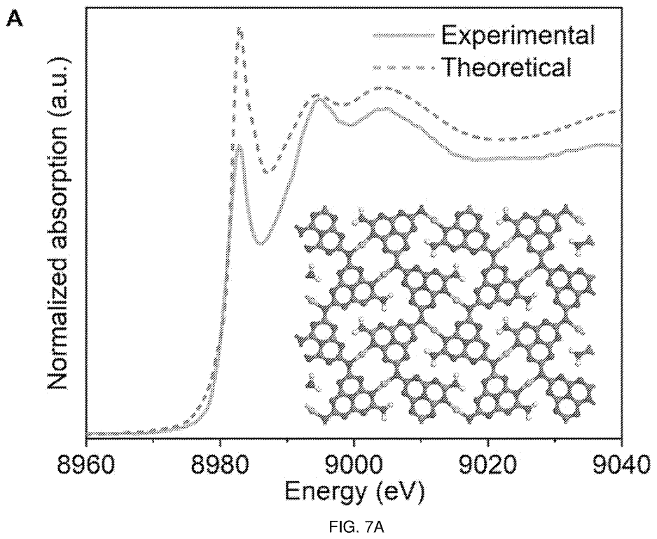

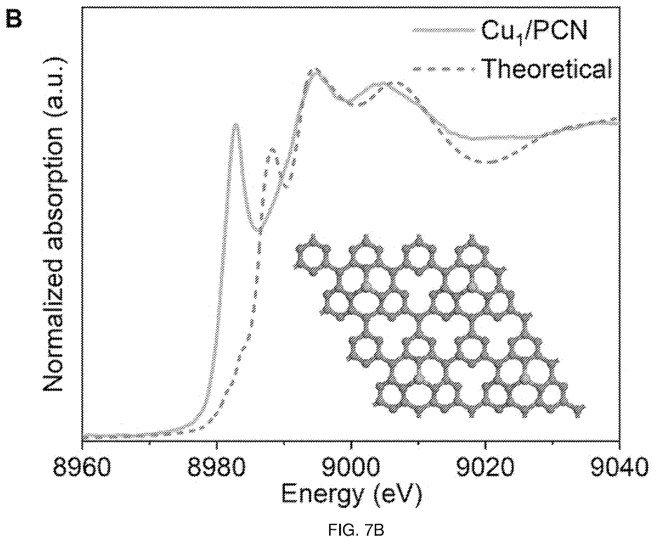

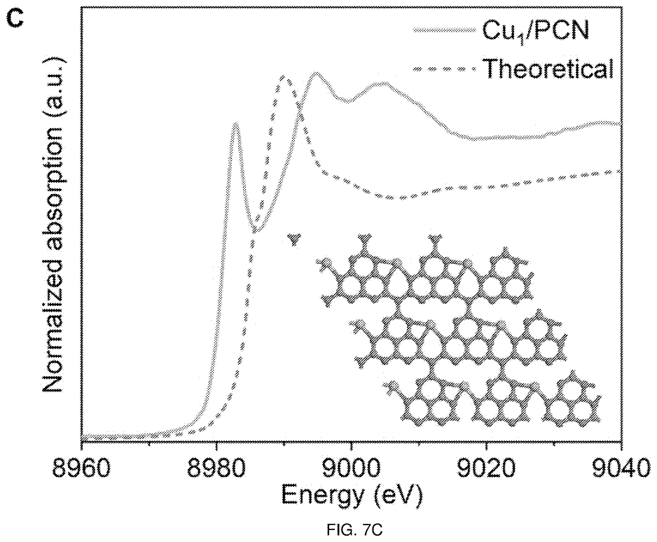

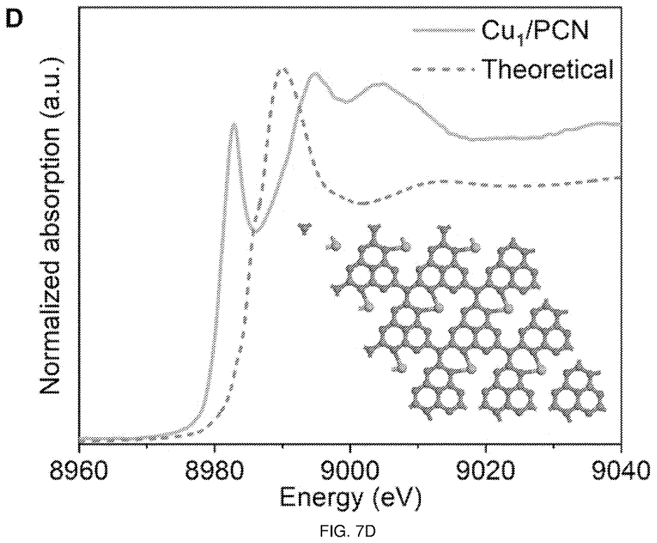

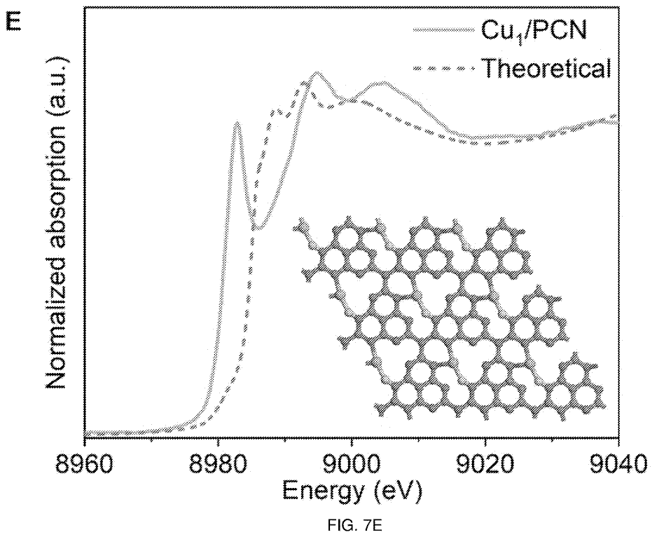

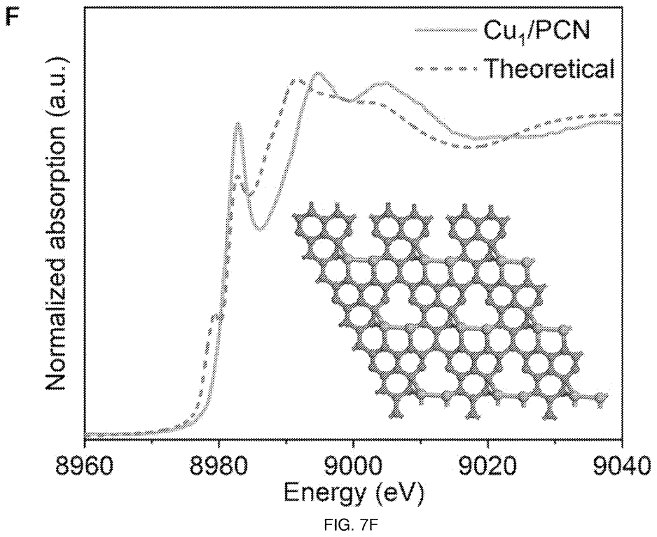

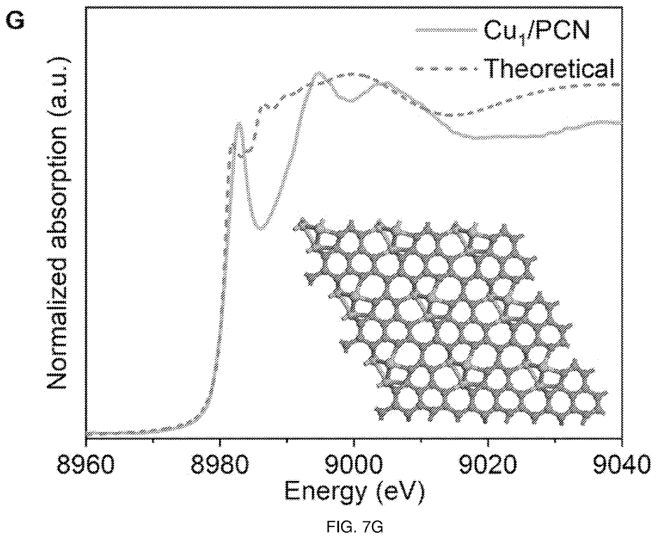

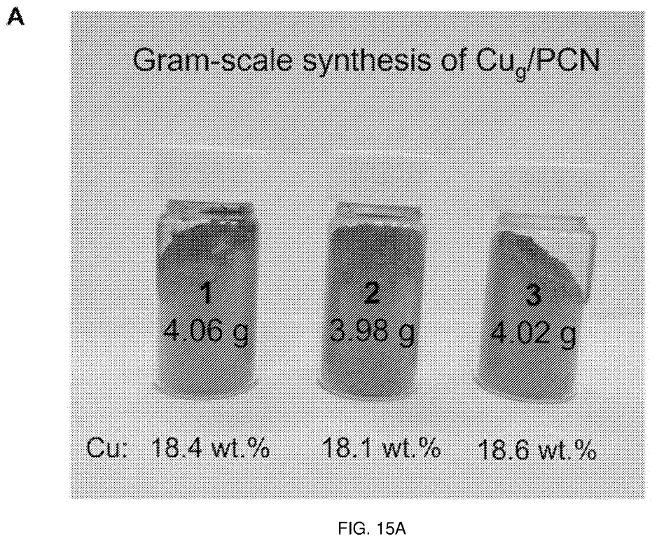

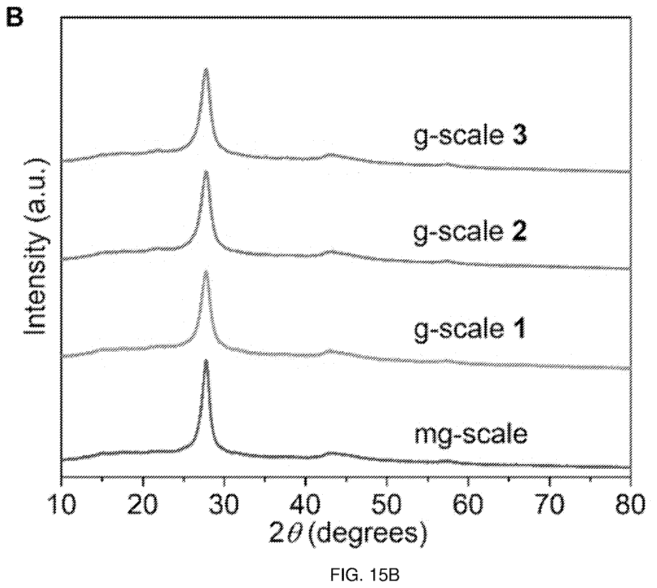

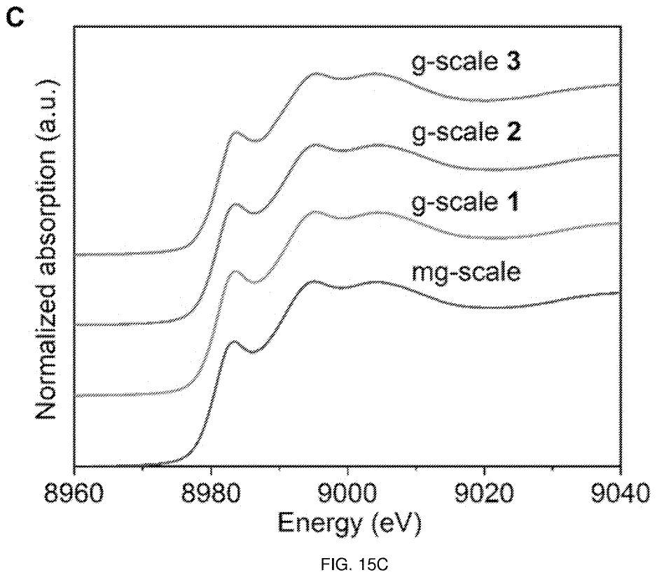

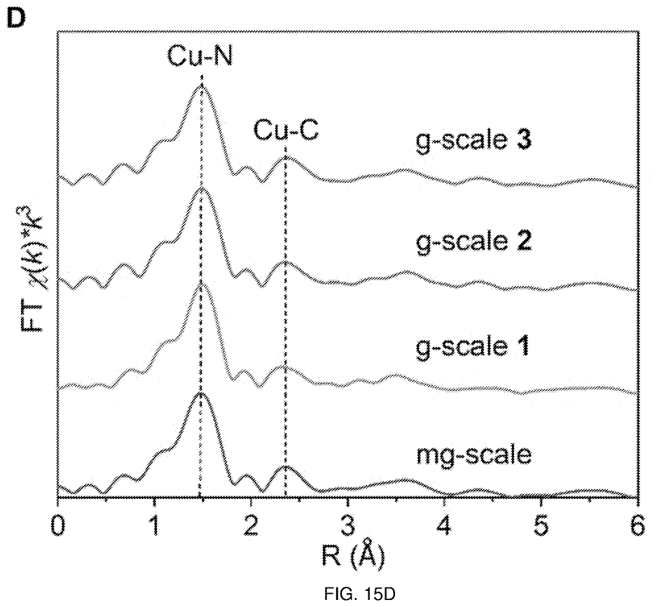

A stepwise ion exchange and ligand removal strategy was devised for the synthesis of low coordinate geminal metal atoms exploiting the abundant and periodic presence of N—H functional groups as metal coordination sites in polymeric carbon nitride (PCN, FIG. 1A and FIGS. 15A, 15B, 15C and 15D). Upon introduction, Cu single atoms replace H atoms, as confirmed by the almost completely reduced intensity of N—H group related vibration bands of PCN (FIG. 1B) and are further stabilized by the opposite N atoms. Annular dark field scanning transmission electron microscopy (ADF-STEM, FIG. 1C) acquired over ultra-high density Cu_{g}/PCN (18.3 wt. % Cu) reveals a high concentration of metal atoms, which tend to form regularly-spaced pairs (termed as geminal Cu) with an average Cu—Cu distance of ~0.4 nm. Such a geminal Cu pair anchored in PCN chain is also captured by in-situ scanning tunnelling microscopy characterization (FIG. 5). Elemental mapping using energy-dispersive X-ray spectroscopy (EDS) and powder X-ray diffraction (XRD) measurements also confirm the uniform metal dispersion and the absence of nanoparticles or clusters in Cu_{g}/PCN (FIGS. 6A, 6B, 6C, 6D, 6E and 6F). Consistently, the Fourier transformed extended X-ray absorption fine structure (EXAFS, FIG. 1D and Table 1) spectrum reveals a dominant feature centered at 1.5 Å and a weak peak at around 2.2 Å, corresponding to the first shell Cu—N and the second shell Cu—C interatomic distance, respectively. A prominent pre-edge feature around 8983 eV in the Cu K-edge X-ray absorption near edge structure (XANES) can be assigned to 1s→4p_{xy} transition, indicative of a diagonal coordinated Cu. To confirm the atomic structure of the Cu sites, first-principle calculations based on density functional theory (DFT) were conducted to identify possible models and simulate their corresponding XANES spectra. Among all the proposed structures (FIGS. 7A, 7B, 7C, 7D, 7E, 7F and 7G), the one consisting of heptazine chains shows the best agreement with experimental Cu K-edge XANES spectrum (FIG. 1E). Such a diagonally coordinated N—Cu—N structure endows a Cu valence state of +1 and this monovalent Cu(I) state is further confirmed by X-ray photoelectron spectroscopy and electron paramagnetic resonance spectroscopy (EPR, FIGS. 7A, 7B, 7C, 7D, 7E, 7F and 7G).

TABLE 1
| Results of EXAFS fits of Cu_{g}/PCN and Cu₁/NC.

| Sample | Shell | N | R (Å) | σ² (10⁻³Å²) | R factor |
|---|---|---|---|---|---|
| Cu_{g}/PCN | Cu—N | 2.5 | 1.90 | 0.0069 | 0.005 |
| Cu₁/NC | Cu—N | 4.2 | 1.95 | 0.0053 | 0.003 |

N, coordination number;
R, distance between absorbing and backscattering atoms;
σ², Debye-Waller factor to account for thermal and structural disorders;
R factor as a measure of the goodness of fit.
The fitted coordination environment of Cu_{g}/PCN, and Cu₁/NC coincides well with the proposed structures from DFT.

### Example 3: Performance in Cross-Coupling Reactions

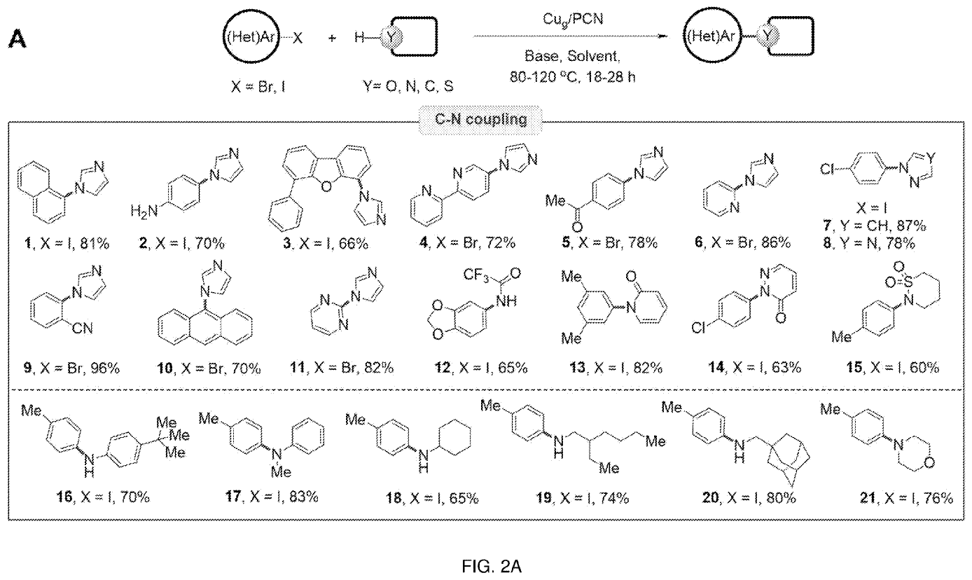

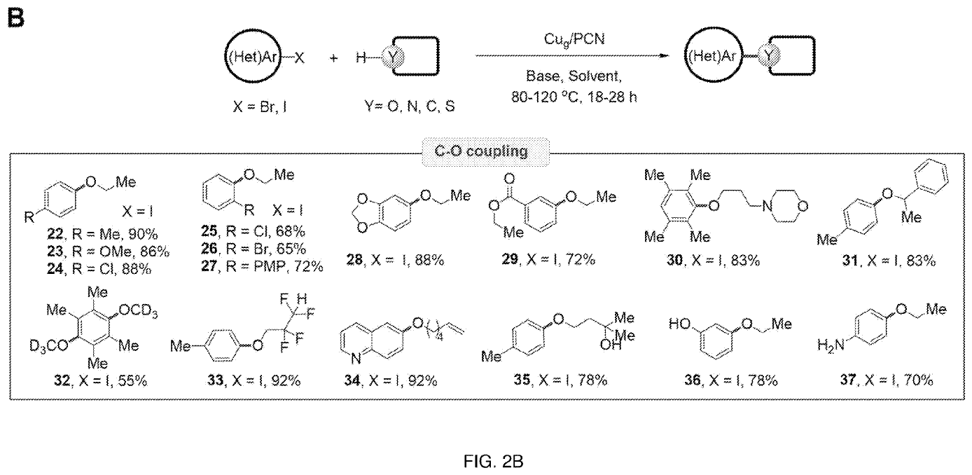

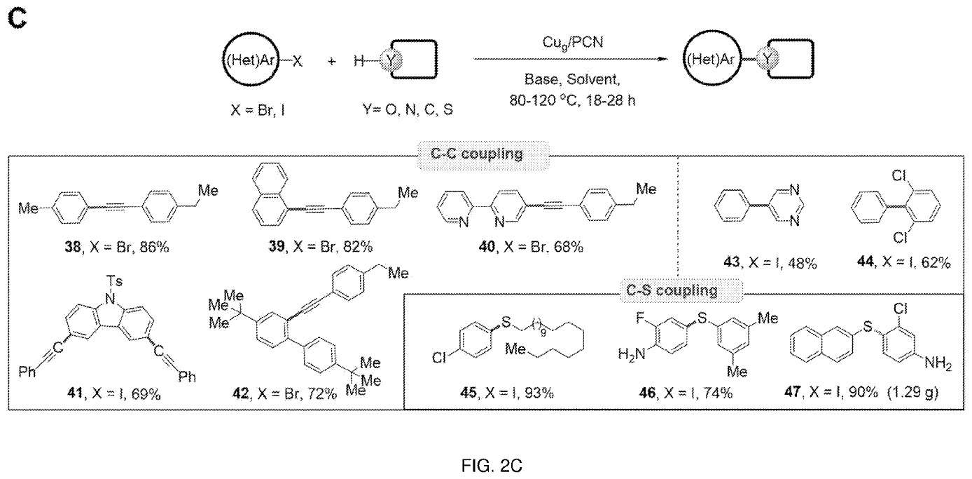

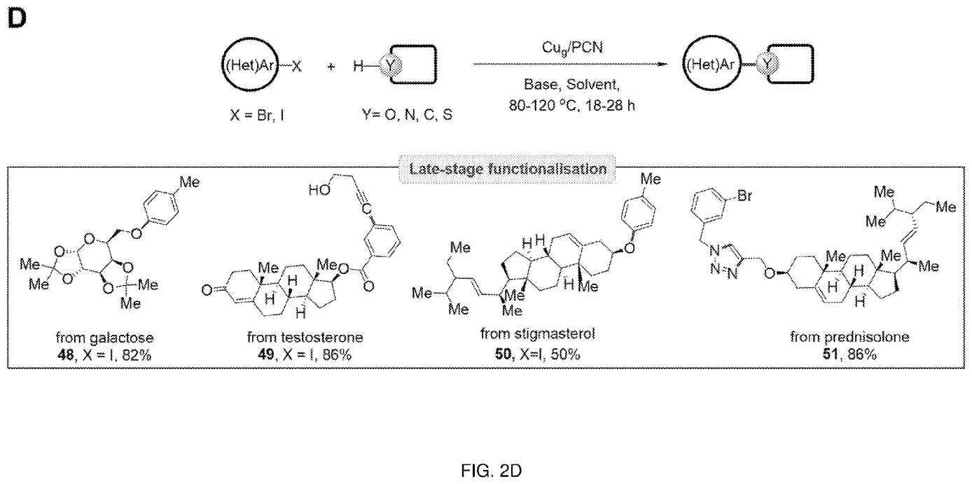

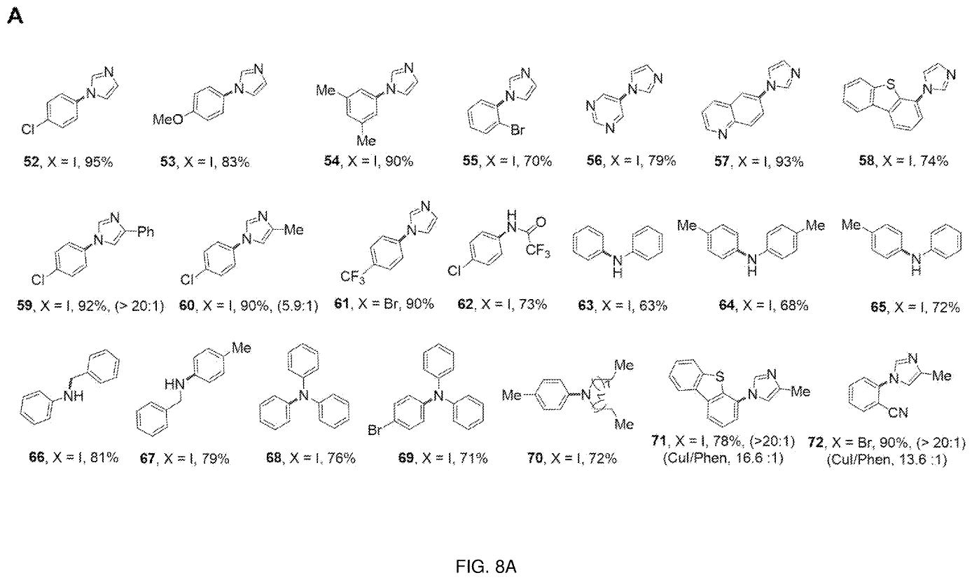

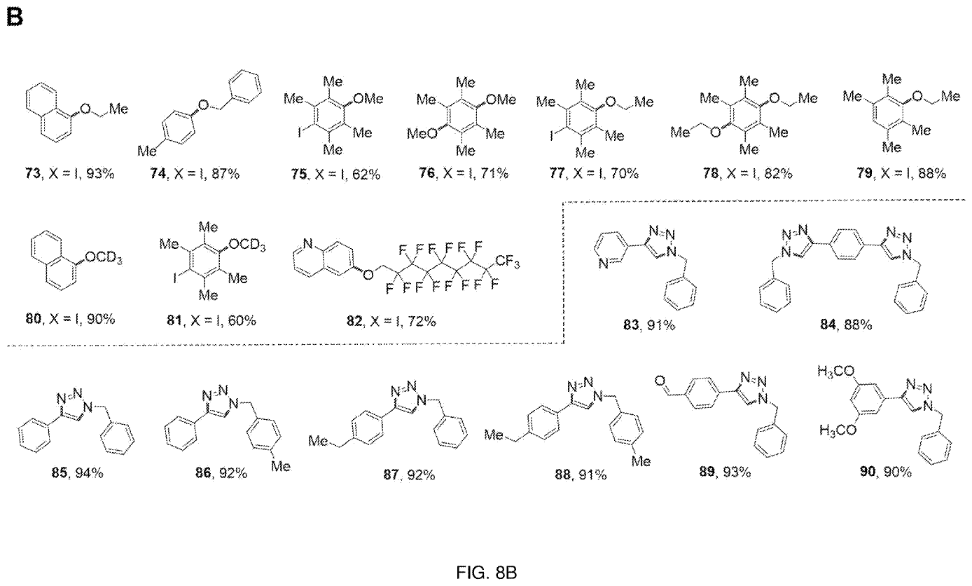

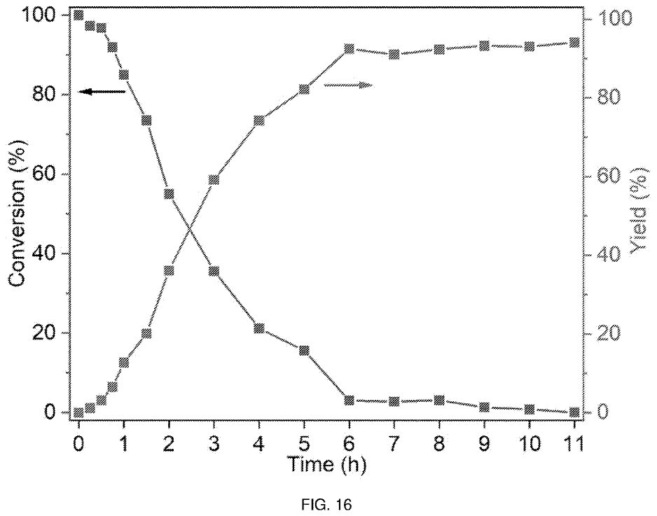

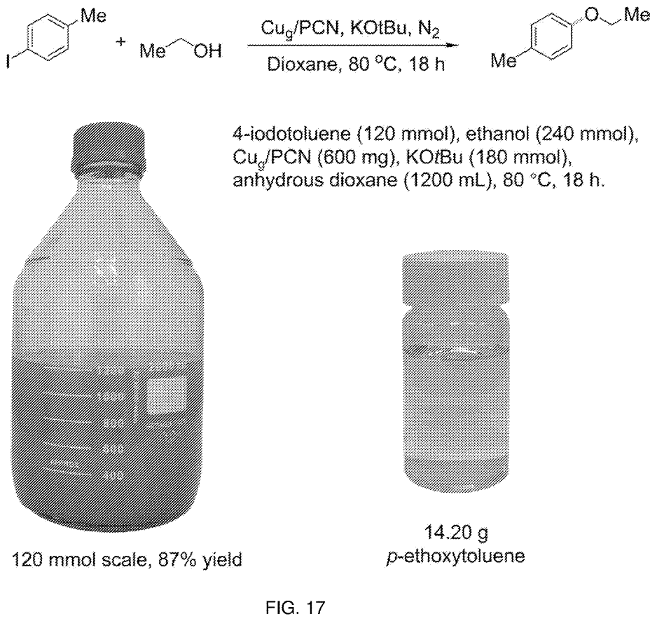

To assess the reaction scope of the Cu_{g}/PCN catalyst, various (hetero)aryl halides with different electronic and/or steric attributes were examined under ligand-free conditions (Table 2 and FIG. 16). The results demonstrate that Cu_{g}/PCN is an efficient heterogeneous catalyst for general cross-coupling reactions, transforming diverse reactants to targeted molecules with high selectivity and yields (FIGS. 2A, 2B, 2C and 2D and FIG. 8A and FIG. 8B). Aryl and heteroaryl iodides and bromides are excellent coupling partners, affording a wide spectrum of products in good to excellent yields (1-82). Electron-donating, electron-withdrawing, functional substituents (e.g., hydroxyl, amino, halide, ester, ketone, nitrile, alkene, alkyne), and heterocycles are well-tolerated, providing great flexibility for further synthetic manipulations. In particular, nitrogen heterocycles (1-11, 52-61), amides (12-14, 62), sulfonamide (15), aryl amines (16, 17, 63-69), and alkyl amines (18-21, 70) all participate in C—N coupling, yielding diverse functionalized nitrogen-containing compounds. Fluorinated alcohols (33, 82) and deuterated methanol (32, 80, 81) are effective substrates for C—O coupling. Synthetically useful C—C coupling using terminal alkynes (38-42) or electron-deficient (hetero)arenes (43, 44) can also be achieved. The GACs system outperforms Cu nanocatalysts and benchmarked homogeneous cuprous iodide at the same molar equivalent of copper (Table 3). Furthermore, Cu_{g}/PCN also demonstrated excellent performance (83-90, 88 to 94% yield) for azide-alkyne cycloaddition, a reaction known to involve two metal centers e.g., described in Worrell, B., Malik, J. & Fokin, V. V. Direct evidence of a dinuclear copper intermediate in Cu (I)-catalyzed azide-alkyne cycloadditions. Science 340, 457-460 (2013), the contents of which are fully incorporated herein by reference. The Cu_{g}/PCN-catalyzed cross-coupling is scalable: production of p-ethoxytoluene (22) was successfully executed on 26-gram scale, affording the C—O coupling product in 87% isolated yield (FIG. 17).

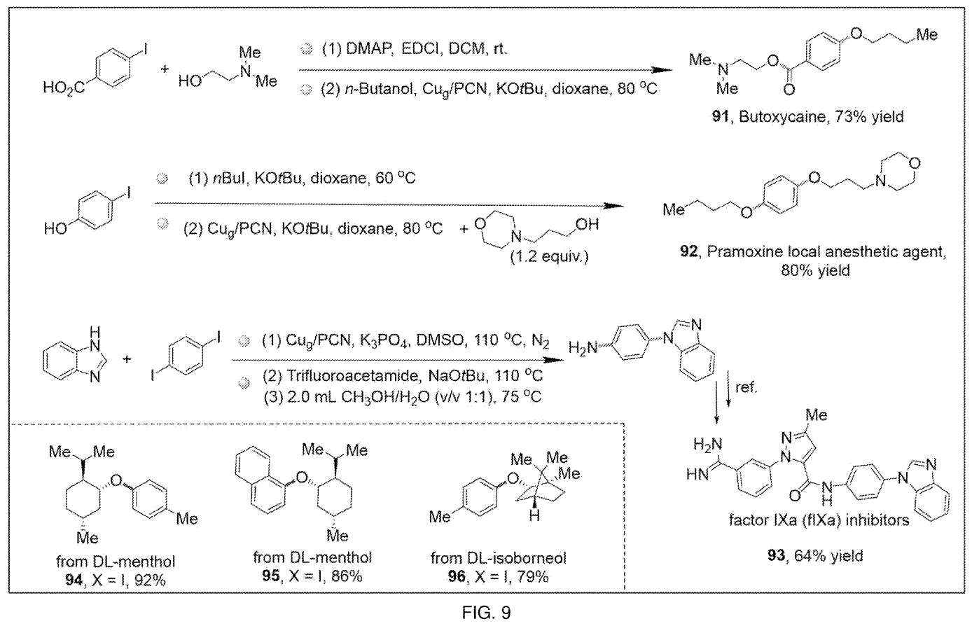

The compatibility of the GACs system was further tested on biorelevant molecules. Representative examples involving the cross-coupling of DL-menthol (94, 95), stigmasterol (50), DL-isoborneol (96), 1,2,3,4-diacetone galactose (48) and the derivatives of testosterone (49) and prednisolone (51) (FIGS. 2A, 2B, 2C and 2D and FIG. 9), as well as the large-scale synthesis of drug-like p-arylthio aniline (47, 1.2 g) smoothly proceeded to deliver the corresponding products, highlighting the Cu_{g}/PCN catalyst's applicability for late-stage modification of complex molecules. Starting from commercially available substrates, batch synthesis of selected pharmaceutically active compounds (91-93) was also successfully accomplished (FIG. 9).

TABLE 2
Optimization of the reaction conditions for C—O coupling. Reactions
were carried out under an N₂ atmosphere. Conditions: 4-iodotoluene
(1.0 equiv.), ethanol (2.0 equiv.), Cu_{g}/PCN Catalyst (1 mg), base
(1.5 equiv.), solvent (2 ml), 80 °C, 18 h, 0.2 mmol scale.

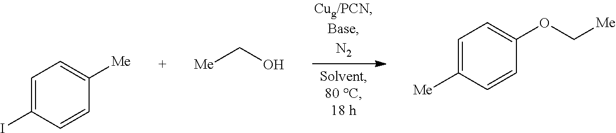 [SMILES: Cc1ccc(I)cc1 ; CCO ; CCOc1ccc(C)cc1]

| Entry | Base | Solvent | Temp. (ºC) | Conversion (%) | Yield (%) |
|---|---|---|---|---|---|
| 1 | KOtBu | dioxane | 80 | 95 | 90 |
| 2 | NaOtBu | dioxane | 80 | 19 | 14 |
| 3 | LiOtBu | dioxane | 80 | <5 | 1.2 |
| 4 | K₃PO₄ | dioxane | 80 | 1 | trace |
| 5 | Cs₂CO₃ | dioxane | 80 | <1 | trace |
| 6 | K₂CO₃ | dioxane | 80 | <1 | trace |
| 7 | KOtBu | Toluene | 80 | 78 | <5 |
| 8 | KOtBu | CH₃CN | 80 | <1 | trace |
| 9 | KOtBu | THF | 80 | 70 | 60 |
| 10 | KOtBu | DMF | 80 | 36 | 28 |

TABLE 3
Comparison of the turnover number (TON) reported over copper catalysts in the Ullmann reaction.

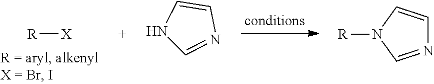

| Entry^{a} | R—X | Catalyst^{b} | Reaction mode | TON | |
|---|---|---|---|---|---|
| 1 | 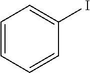 | Cu₂O + L₁ | homogeneous | 36.8 | CE |
| 2 | 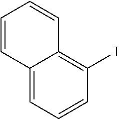 | Cu₂O + L₁ | homogeneous | 18.6 | CE |
| 3 | 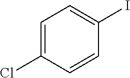 | CuBr + L₂ | homogeneous | 9.1 | CE |
| 4 | 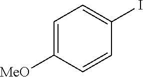 | CuI + L₃ | homogeneous | 49 | CE |
| 5^{a} | 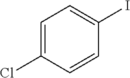 | CuCl + L₄ | homogeneous | 18.4 | CE |
| 6 | 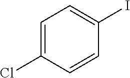 | CuI + furfuryl alcohol | homogeneous | 80 | CE |
| 7 | 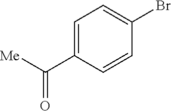 | Cu₂O + L₁ | homogeneous | 18.0 | CE |
| 8 | 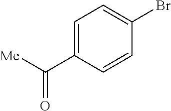 | CuI + L₅ | homogeneous | 4.7 | CE |
| 9 | 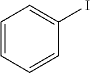 | Cu^{l-}USY | heterogeneous | 8.8 (Recycle 5 times) | CE |
| 10 | 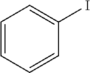 | CuI/Meso-N-C | heterogeneous | 5.9 | CE |
| 11 | 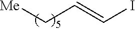 | sub-nanometric Cu clusters | heterogeneous | 413 | CE |
| 12 | 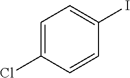 | Cu_{g}/PCN | heterogeneous | 2352 | This work |
| 13 | 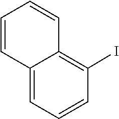 | Cu_{g}/PCN | heterogeneous | 1443 | This work |
| 14 | 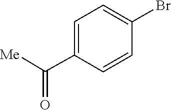 | Cu_{g}/PCN | heterogeneous | 1556 | This work |

^{a}Entries 1-10 estimated from reported product yield
^{b}L₁ = 4,7-Dimethoxy-1,10-phenanthroline; L₂ = β-Keto ester; L₃ = 8-hydroxyquinalidine; L₄ = (1E,2E)-oxalaldehyde dioxime; L₅ = per-6-amino-β-cyclodextrin; TON = [mole of converted aryl halides/[mole of Cu catalyst] × 100%;
CE: Comparative Example.

### Example 4: Reaction Mechanism

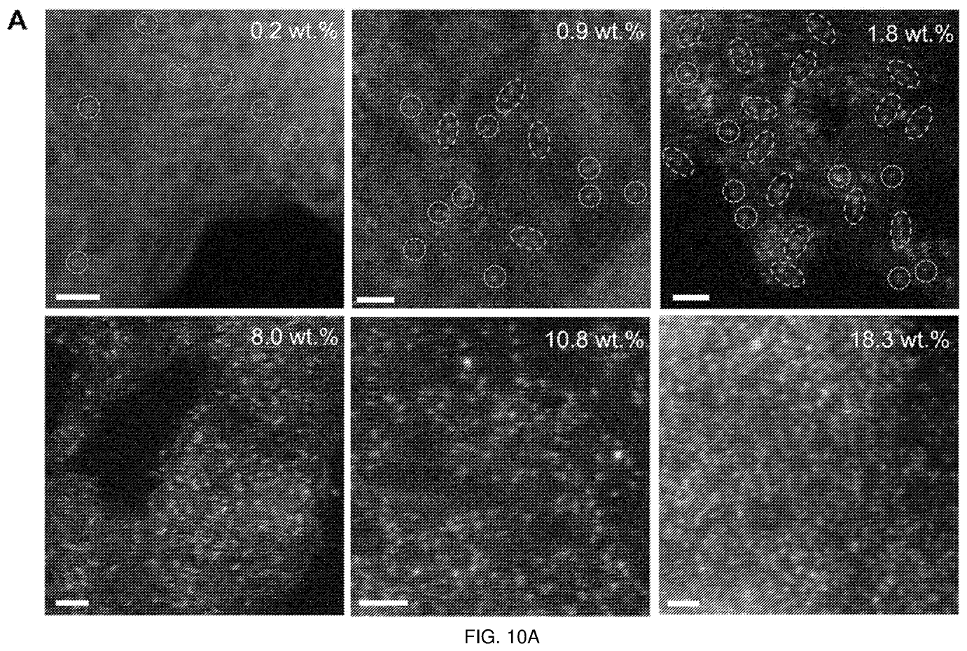

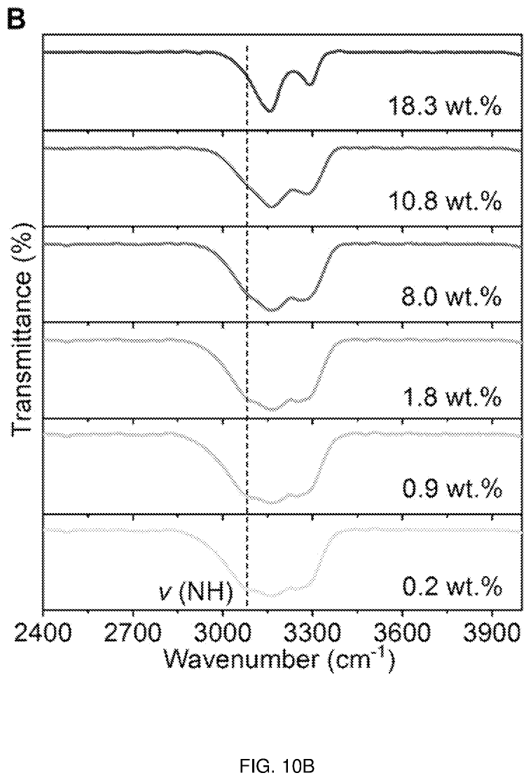

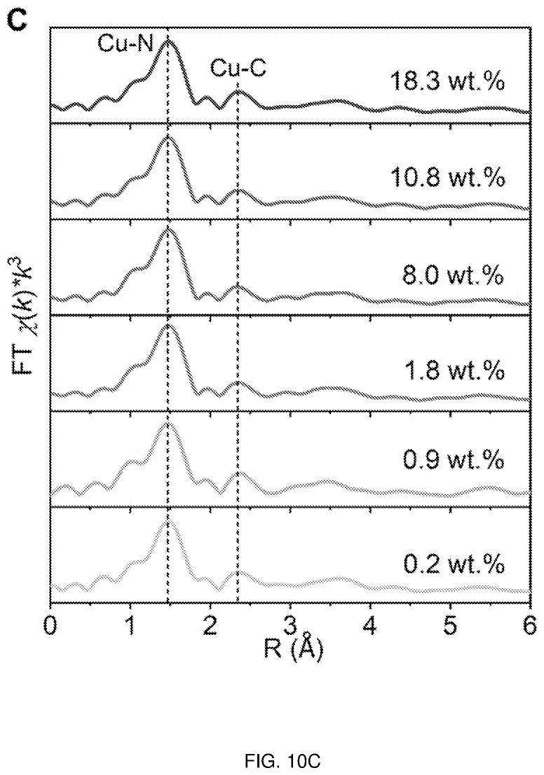

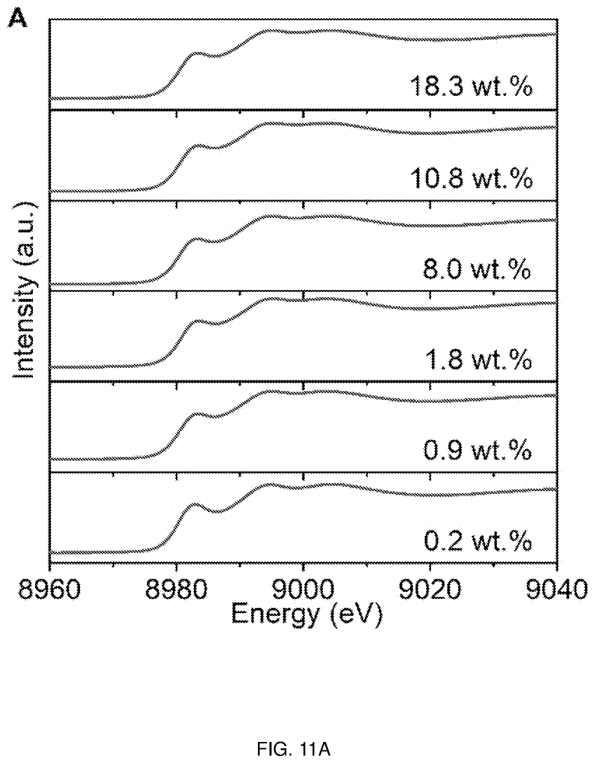

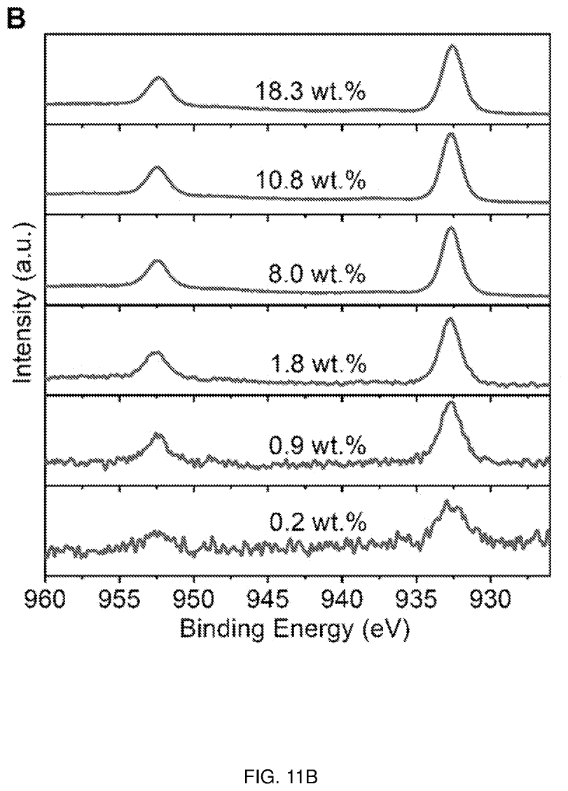

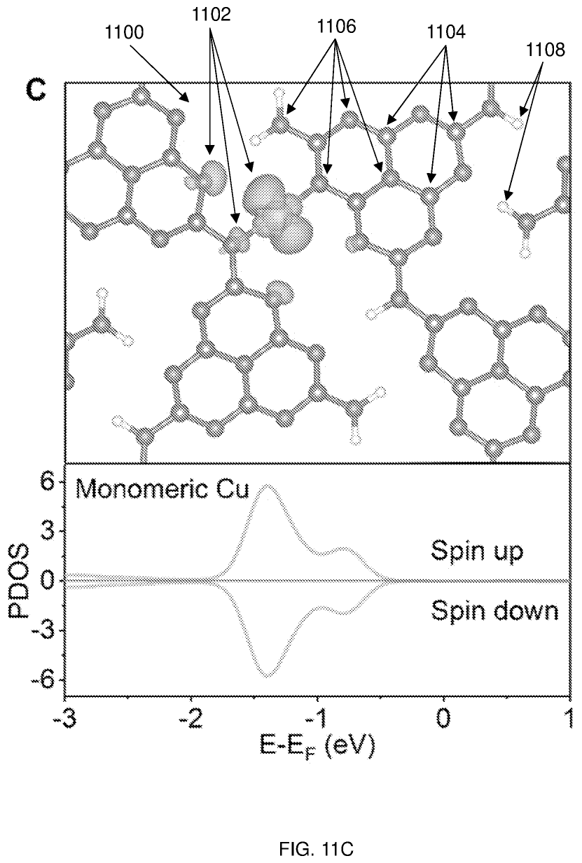

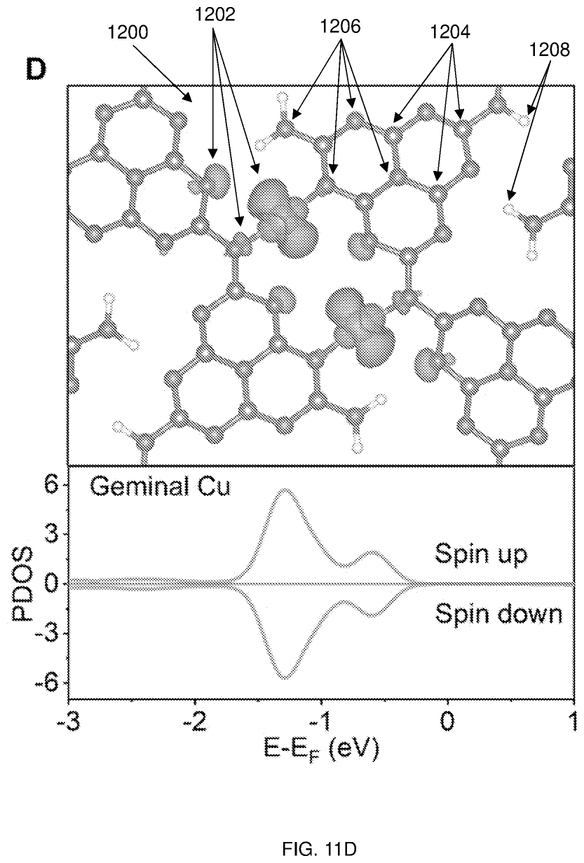

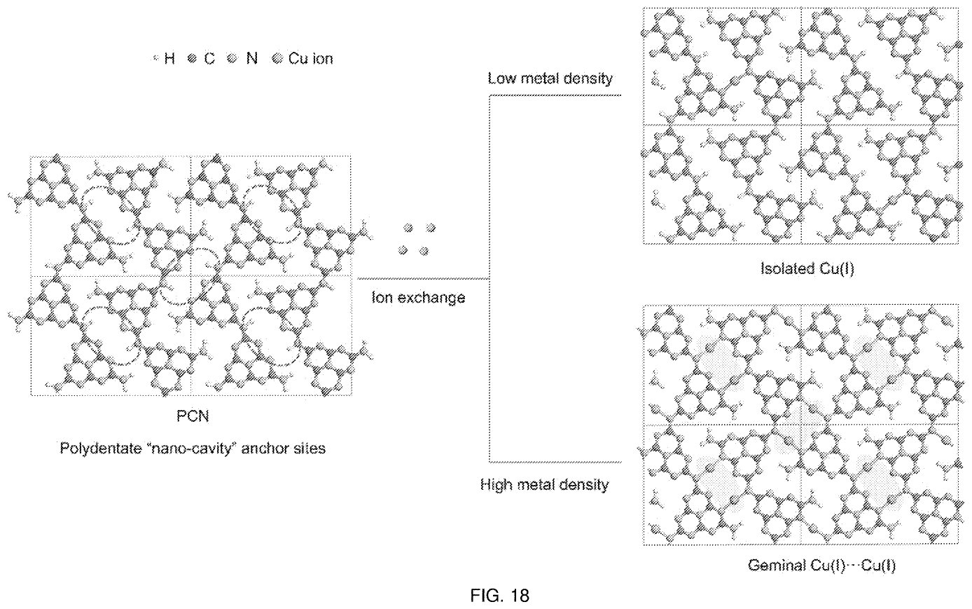

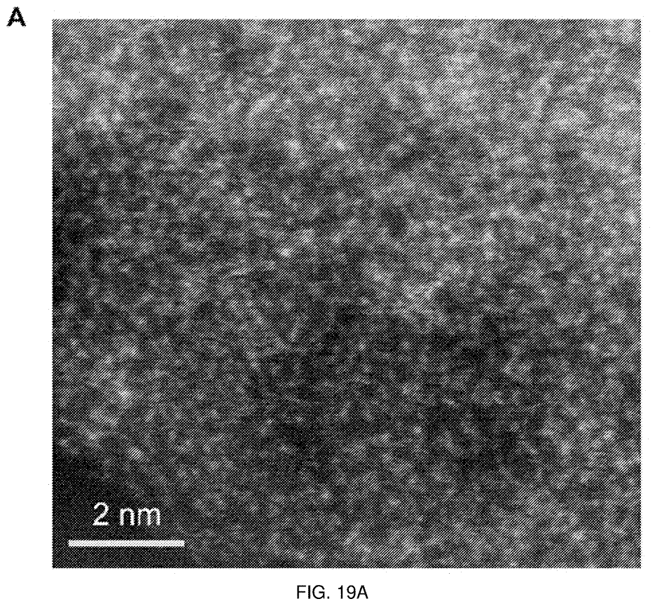

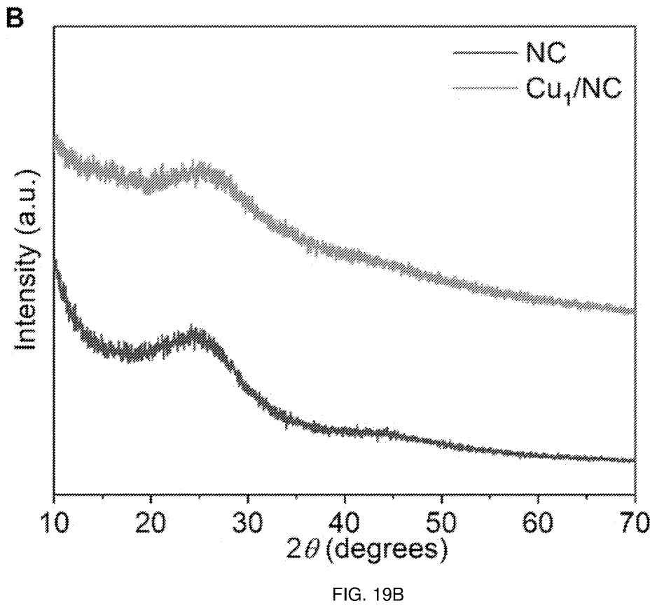

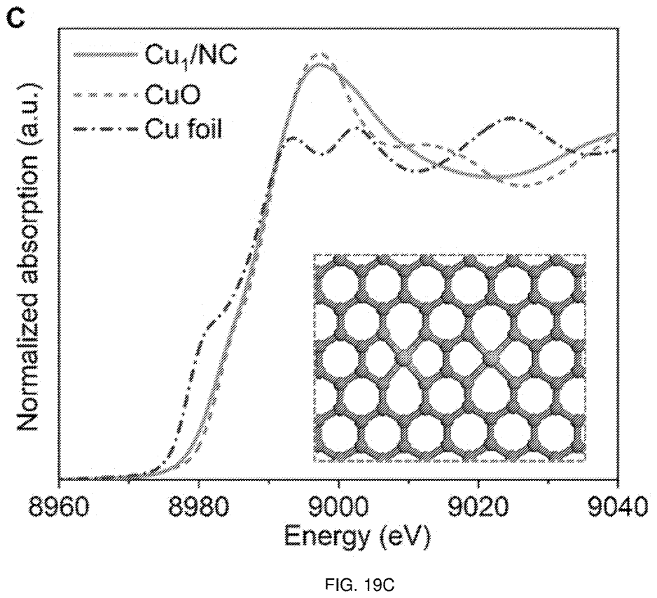

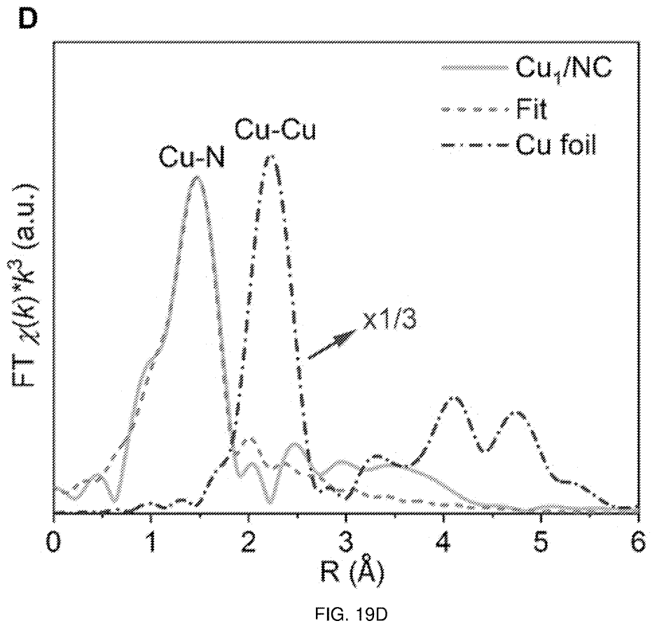

To confirm the unique site cooperativity in the GACs, a series of Cu single-atom catalysts (Cu₁/PCN) was prepared with Cu contents ranging from 0.2-18.3 wt. %, where the ratio of monomeric Cu(I) to geminal Cu(I) . . . Cu(I) sites is expected to decrease upon increasing the Cu content in the sample (FIG. 18). Meanwhile, detailed characterization (FIGS. 10A, 10B and 10C) and quantum-theoretical studies based on DFT calculations (FIGS. 11A, 11B, 11C and 11D) reveal the identical chemical state of each Cu regardless of the metal content. The catalytic performance of Cu₁/PCN catalysts with different Cu contents for C—N, C—O cross-coupling and azide-alkyne cycloaddition were evaluated using the same amount of Cu (2 mol %), respectively (Table 4). Similar trends were observed in all cases: the product yields correlate with the metal content increasing from zero in the sample containing 0.2 wt. % Cu, where all the Cu(I) sites are isolated monomers to around 90% over the catalyst with the highest content of geminal sites. This control experiment reveals the inability of a monomer Cu(I) to trigger the coupling reaction because of the high energy barrier for oxidation of low-valent copper to a trivalent Cu(Ill) intermediate, highlighting the necessity of a geminal Cu(I) . . . Cu(I) structure. In addition, the low valence state of the metal centre endowed by the diagonally coordinated N—Cu—N configuration also plays an important role in the activation of reactants. This is in stark contrast to zero activity observed over ultra-high-density divalent single atom Cu(II) on nitrogen-doped carbon for the C—O coupling reaction, even if the catalyst contains an abundance of adjacent CuN₄ sites (FIGS. 19A, 19B, 19C and 19D).

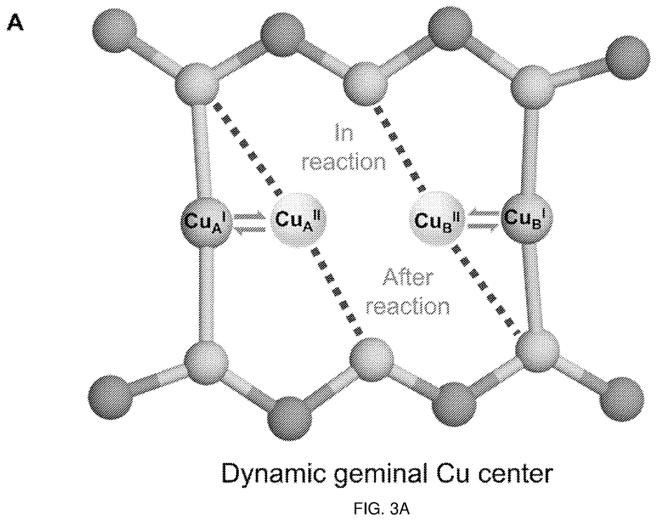

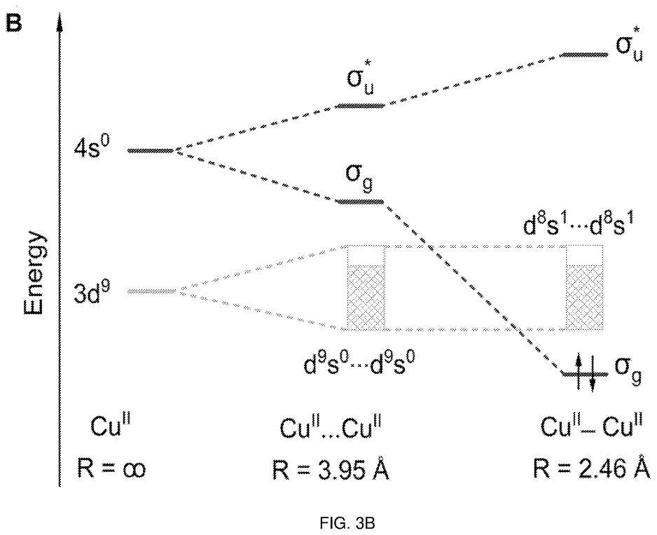

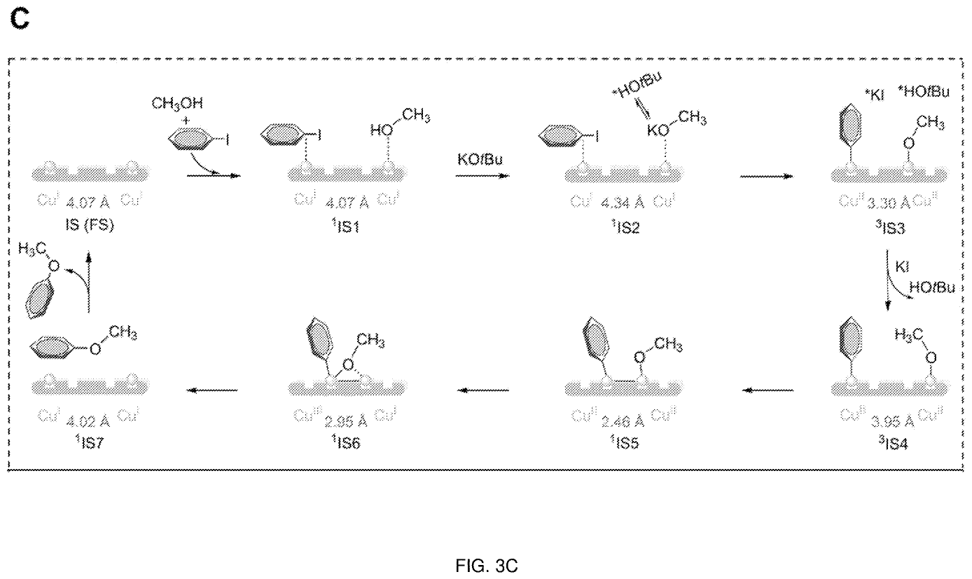

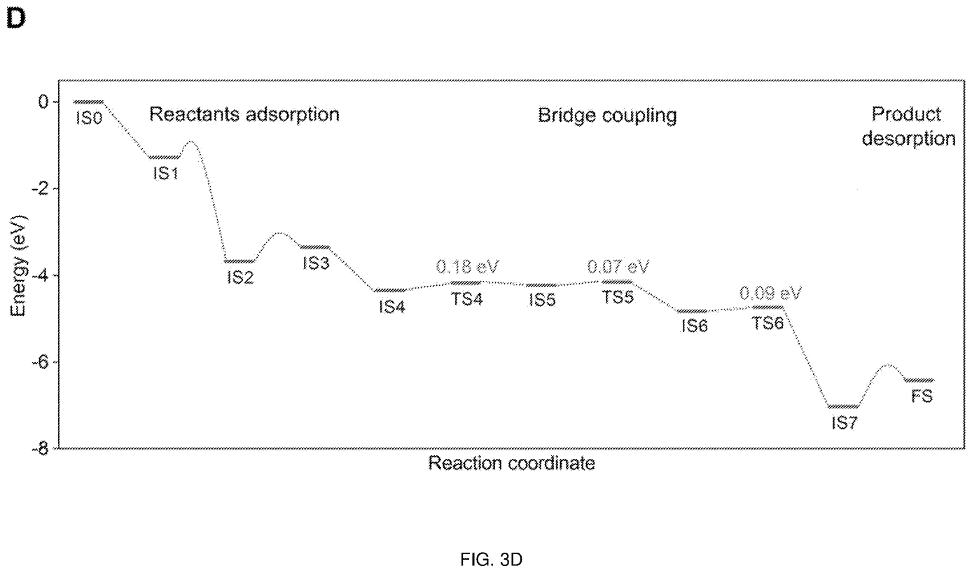

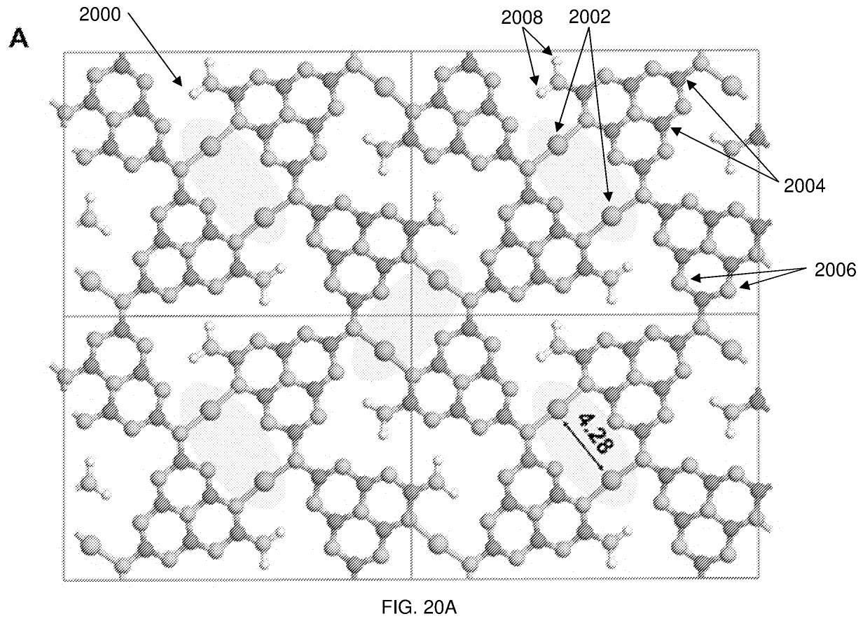

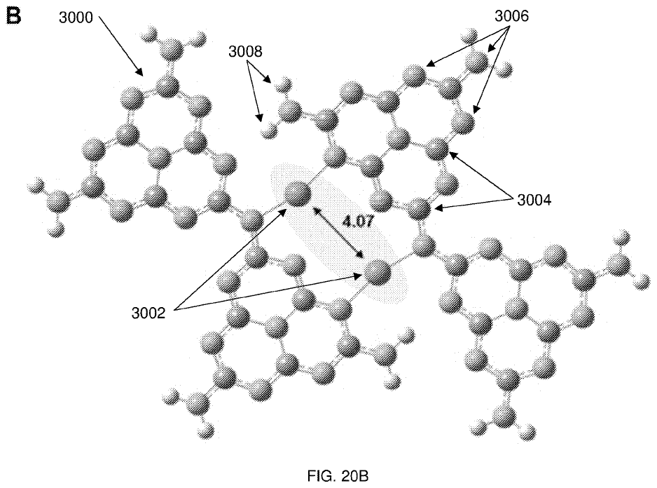

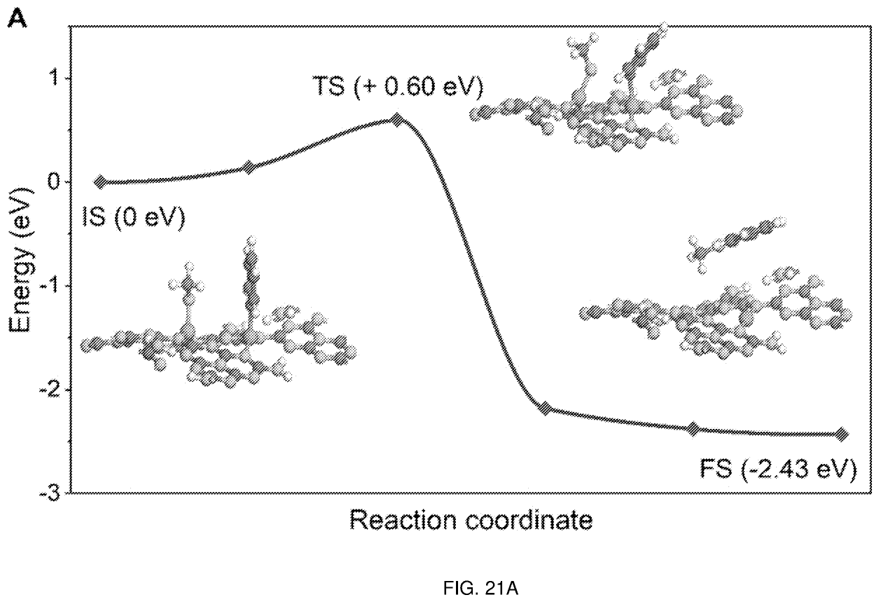

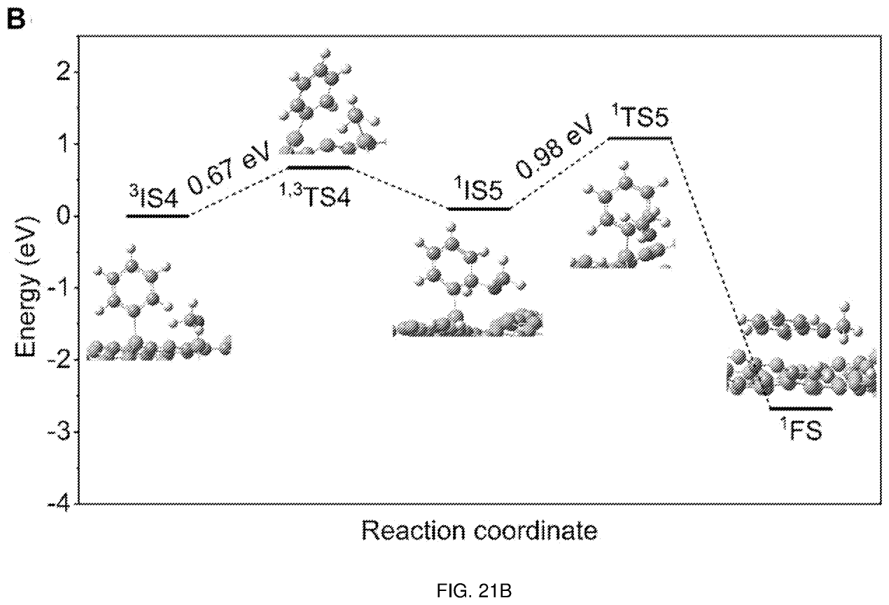

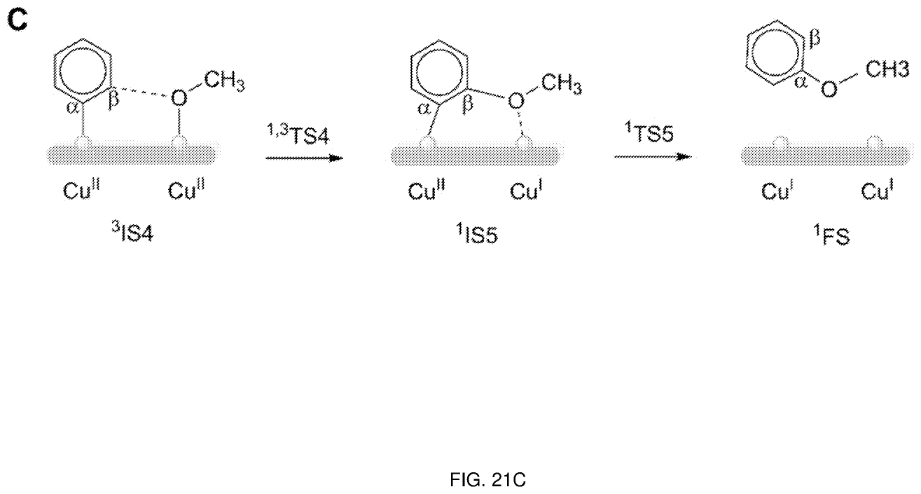

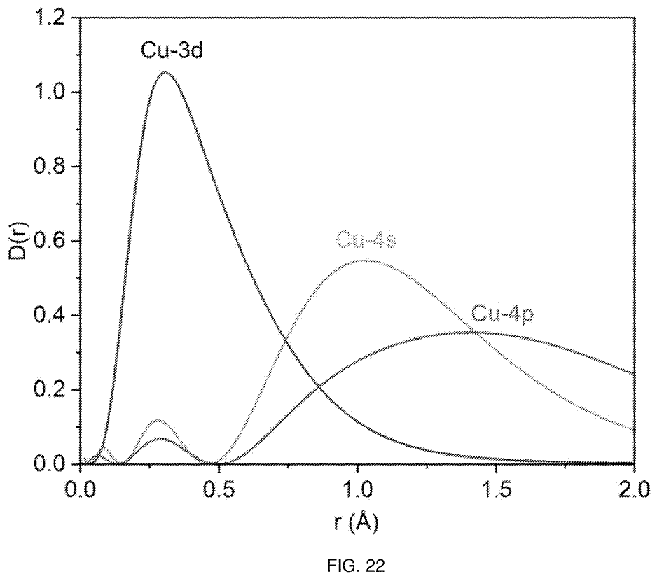

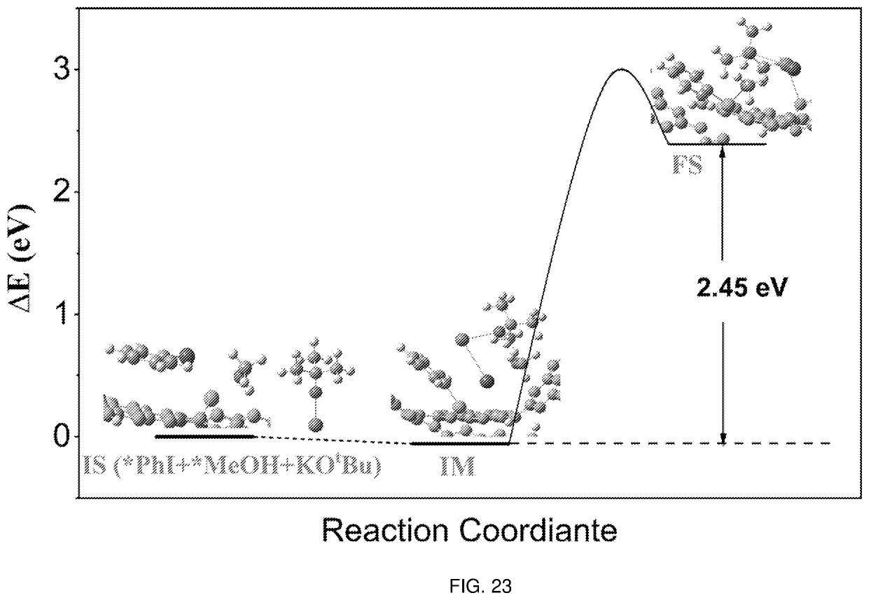

Given the well-documented difficulty in oxidative addition on Cu, the study performed quantum-theoretical investigations (periodic and cluster models, FIGS. 20A and 20B) to explore the coupling of 4-iodobenzene with methanol to gain molecular-level understanding of the cross-bond formation mediated by Cu_{g}/PCN. Initiation is induced by the separate adsorption of (hetero)aryl halide (4-iodobenzene) and the partner nucleophilic reactant (methanol) over each Cu(I) atom of the geminal site. Comprehensive theoretical calculations on the plausible mechanisms strongly support the preference of a direct coupling pathway, wherein a dynamically formed Cu₂ dimer with direct Cu—Cu bonding facilitates C—O bond formation through oxidation addition and subsequent elimination to yield C₆H₅OCH₃ (FIGS. 21A, 21B and 21C). The unique heptazine chain structure allows for the adaptive migration of the geminal Cu (named Cu_{A} and CU_{B}) toward each other upon the adsorption of reactants (FIG. 3A and Table 5), bringing the two reactants (*C₆H₅ and *OCH₃) close enough for the direct cross-bond formation. The system naturally returns to the original static state with unbonded Cu(I) . . . Cu(I) sites after desorption of the product (FIG. 3C and FIG. 3D). Such a dynamic reconfiguration of geminal Cu sites during reaction is expected to be energetically costly, which can be largely offset by electronic energy gain through Cu—Cu bonding orbital interaction. Due to quantum primogenic effect, the atomic radial orbital extension of Cu 4s (~2.2 Å) is much larger than Cu 3d (~1.3 Å) in the radial probability distribution (FIG. 22), so that the Cu₂ dimer formation is energetically compensated via Cu—Cu 4s-4s bonding. Indeed, chemical bonding analysis shows that the 4s-4s orbital interaction between the geminal Cu(II) will cause the system to undergo a potential-energy-surface crossing from Cu^{II}(d⁹s⁰) . . . Cu^{II}(d⁹s⁰) triplet to Cu^{II}(d⁸s¹)-Cu^{IIO}d⁸s¹) singlet states when the direct 4s-4s s-bond is formed (FIG. 3B). Furthermore, DFT calculations indicate that the alternative pathway involving the simultaneous oxidative addition of two reactants over a single copper has a much higher energy barrier (>2.45 eV), thus ruling out the possibility of a cross-coupling reaction occurring over an isolated monoatomic copper (FIG. 23).

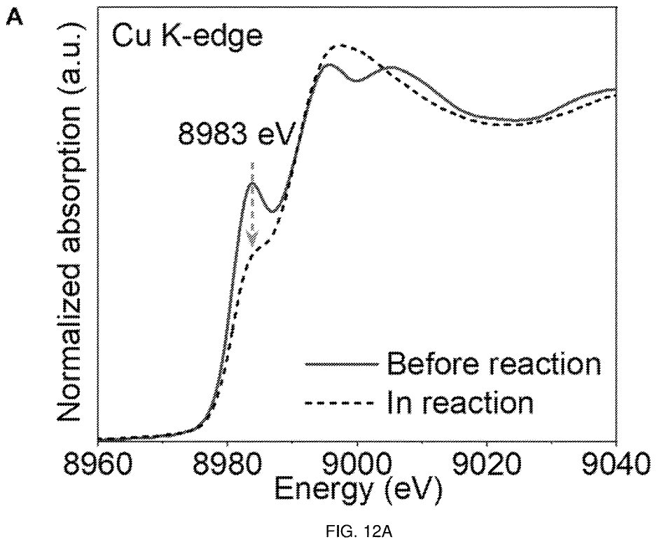

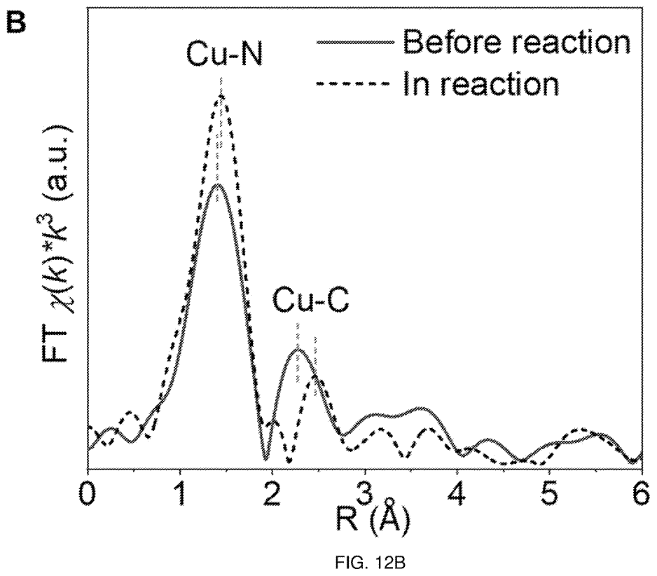

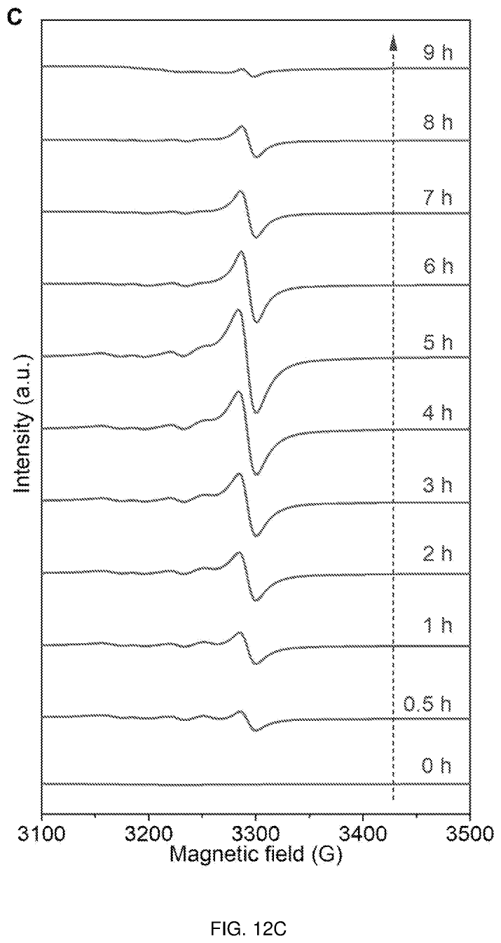

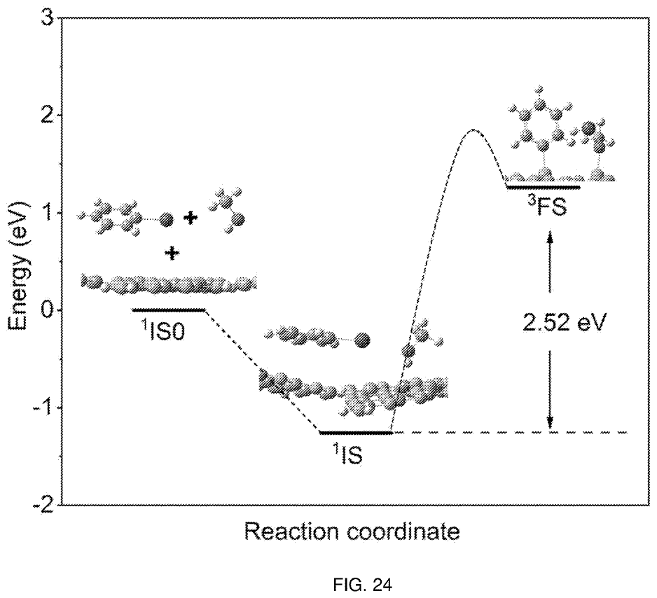

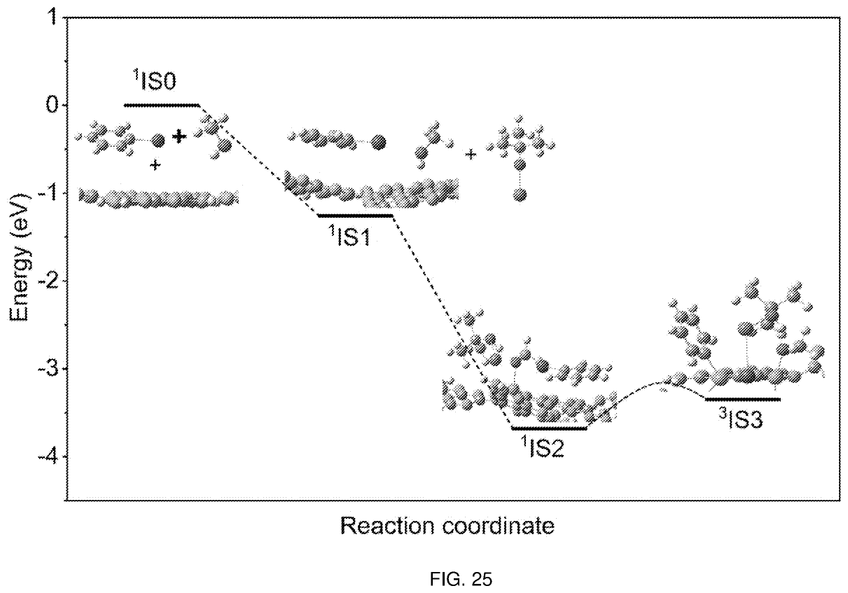

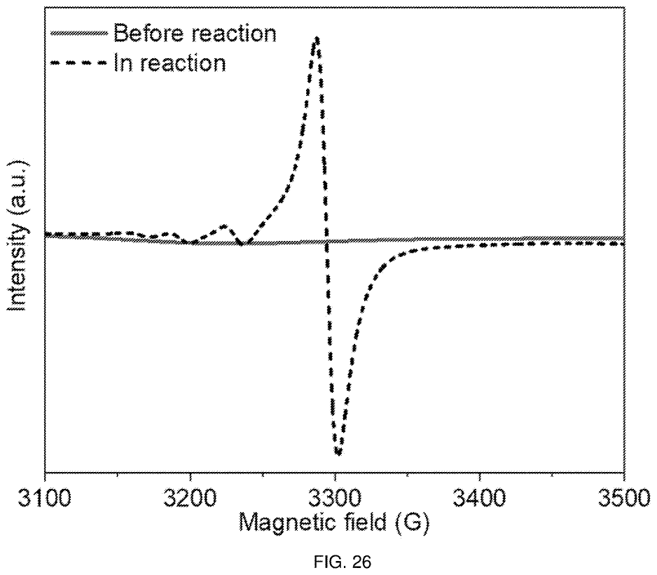

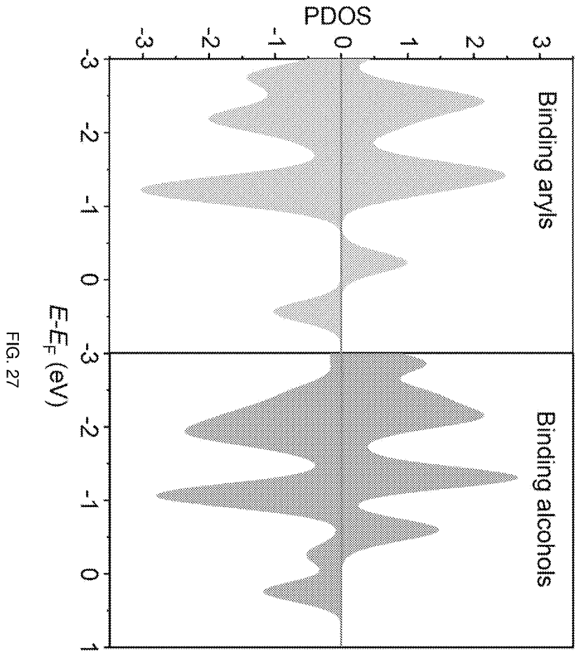

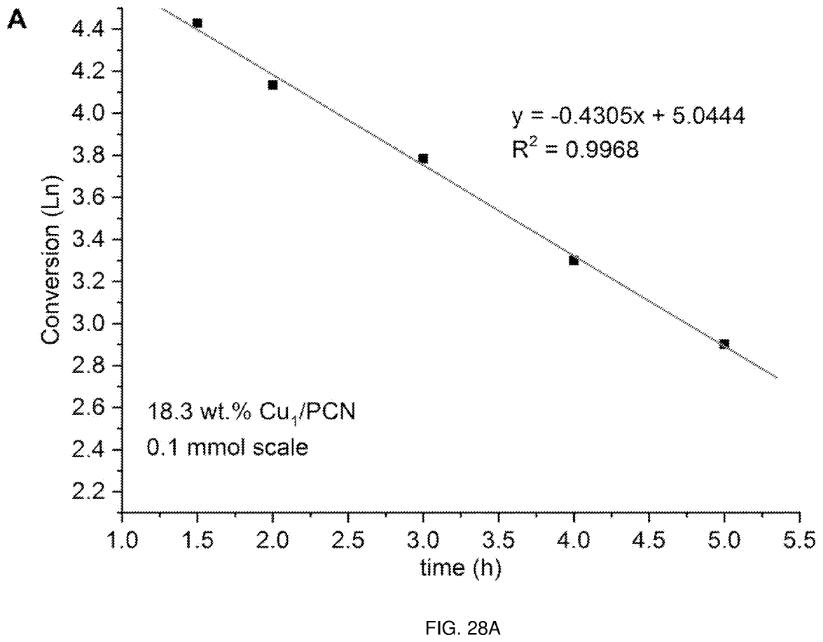

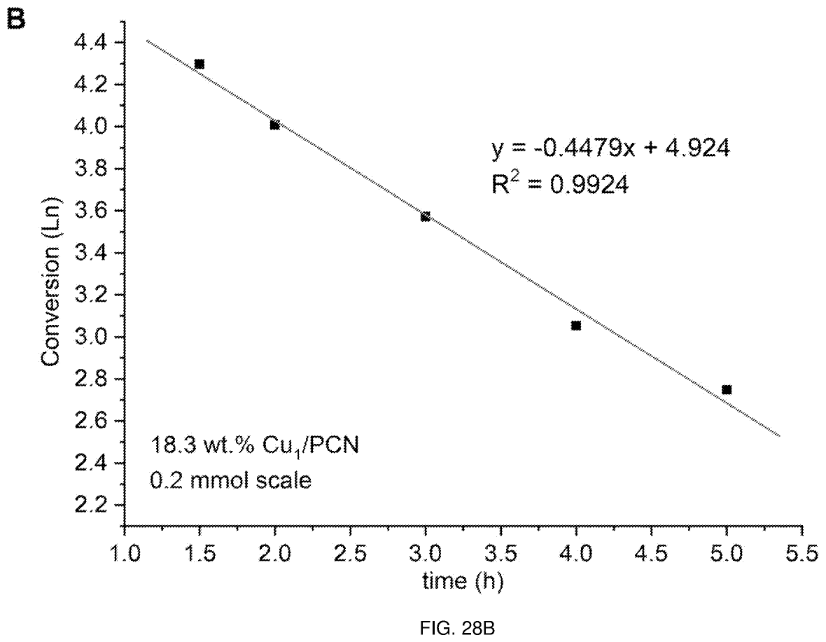

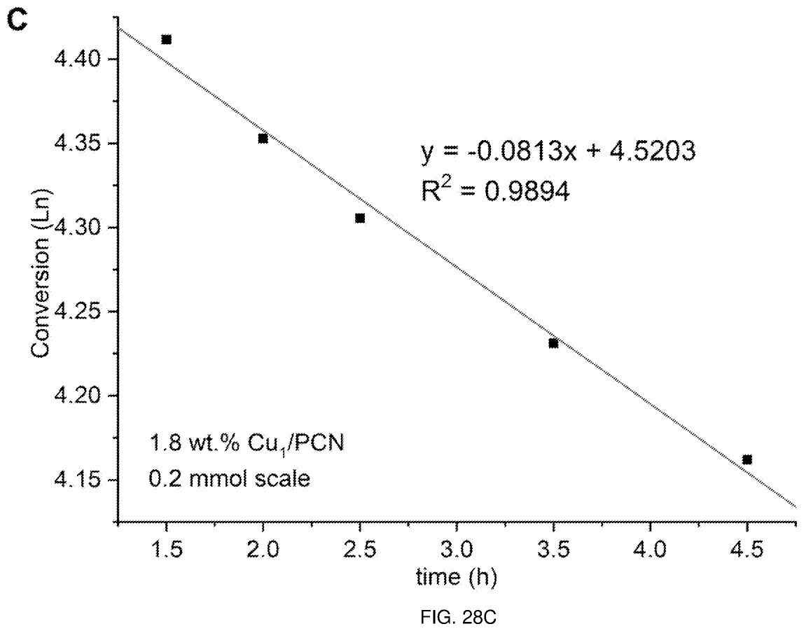

Apart from this, our theoretical calculations also reveal that the adsorption state of individual Cu sites in GACs exhibits negligible mutual influence, distinct from the conventional static dual-atom or bimetallic complex catalysts with a strong direct or indirect metal-metal interaction. More importantly, the co-adsorption process can only occur under the combination of 4-iodobenzene with methanol (neither two 4-iodobenzene nor two methanol molecules), which can explain why only cross-coupling products were obtained instead of homo-coupling products in the C—O coupling reaction (FIG. 24 and FIG. 25). The adsorption of 4-iodobenzene and methanol on Cu sites caused the crucial Cu(I)-to-Cu(II) valence-state conversion, which is directly evidenced by EPR spectroscopic measurements (FIG. 26), where the EPR-silent Cu(I, d¹⁰s⁰) in Cu_{g}/PCN is oxidized to EPR-sensitive Cu(II, d⁹s⁰). Further analysis of the calculated PDOS (FIG. 27) and variation in the Bader charges (Table 6) and spin density population of Cu in Cu_{g}/PCN confirms that the valence state of Cu after absorbing *C₆H₅ or *CH₃O become Cu(II), consistent with the EPR result. In addition, the calculated energy profile reveals the activation of 4-iodobenzene with an activation barrier larger than 0.98 eV, represents the rate-determining step for C—O coupling, consistent with the experimental results (FIGS. 28A, 28B and 28C). The proposed theoretical framework was further validated through in-situ XAFS and EPR experiments (FIGS. 12A, 12B and 12C), which efficiently track the chemical state and local bonding environment evolution of Cu species during the whole reaction cycle.

TABLE 4
| Catalytic performance of Cu₁/PCN with variable
copper contents ir different reactions.

| Cu | Cat. | Cu | C—N | C—O | Azide-alkyne |
|---|---|---|---|---|---|
| content | Amount | (mol | couplingª | coupling^{b} | cycloaddition |
| (wt. %) | (mg) | %) | yield (%) | yield (%) | yield (%) |
| 18.3 | 1.4 | 2 | 95 | 92 | 94 |
| 10.8 | 2.4 | 2 | 76 | 70 | 83 |
| 8.0 | 3.2 | 2 | 56 | 52 | 63 |
| 1.8 | 14.2 | 2 | 21 | 31 | 38 |
| 0.9 | 28.4 | 2 | 3 | 8 | 12 |
| 0.2 | 128.0 | 2 | 0 | 0 | 0 |

^{a}1-Chloro-4-iodobenzene (0.2 mmol), imidazole (0.24 mmol), Cu₁/PCN (2 mol % Cu), K₃PO₄ (0.4 mmol), decane (0.2 mmol), anhydrous DMSO (2.0 mL), 110 ° C, 28 h.
^{b}4-iodotoluene (0.2 mmol), ethanol (0.4 mmol), Cu₁/PCN (2 mol % Cu), KOtBu (0.3 mmol), decane (0.2 mmol), anhydrous dioxane (2.0 mL), 80 °C, 18 h.
^{c}Ethynylbenzene (0.2 mmol), 1-(Azidomethyl)-4-methylbenzene (0.6 mmol), Cu₁/PCN (2 mol % Cu), decane (0.2 mmol), 1:1 water/acetonitrile (3.0 mL), 60 °C, 24 h.

The yield was determined by gas chromatography.

TABLE 6
| Charge density (ΔQ in e⁻) analysis of Cu atoms in
Cu_{g}/PCN at various stages of the C−O coupling reaction.
Initial state (IS), transition state (TS) and
final state (FS) are defined same as presented in FIG. 21A.

| ΔQ(e⁻) | Cu (CH₃O*) | Cu (C₆H₅*) |
|---|---|---|
| Cu_{g}/PCN | −0.60 | −0.60 |
| IS | −0.92 | −0.76 |
| TS | −0.92 | −0.79 |
| FS | −0.60 | −0.60 |

### Example 5: Distinct Advantages of Geminal-Atom Catalysis

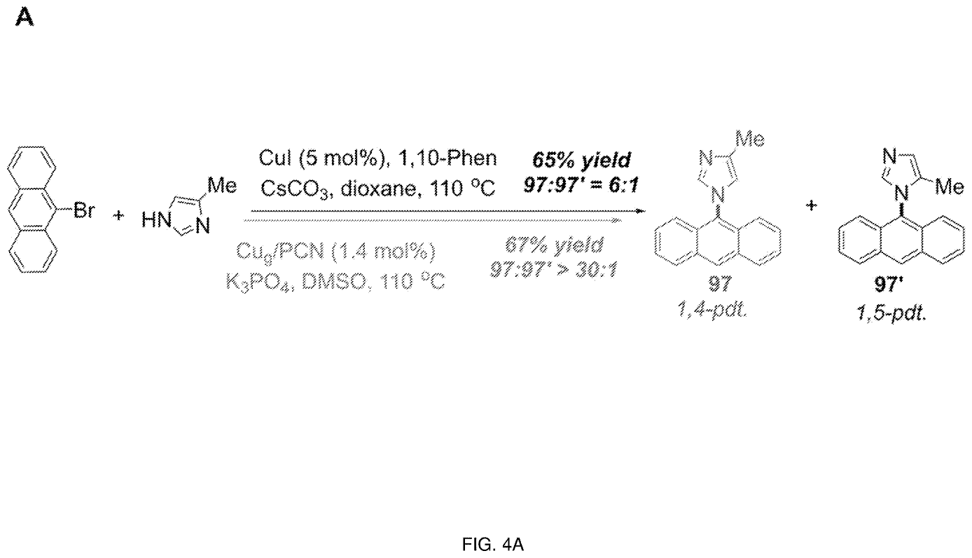

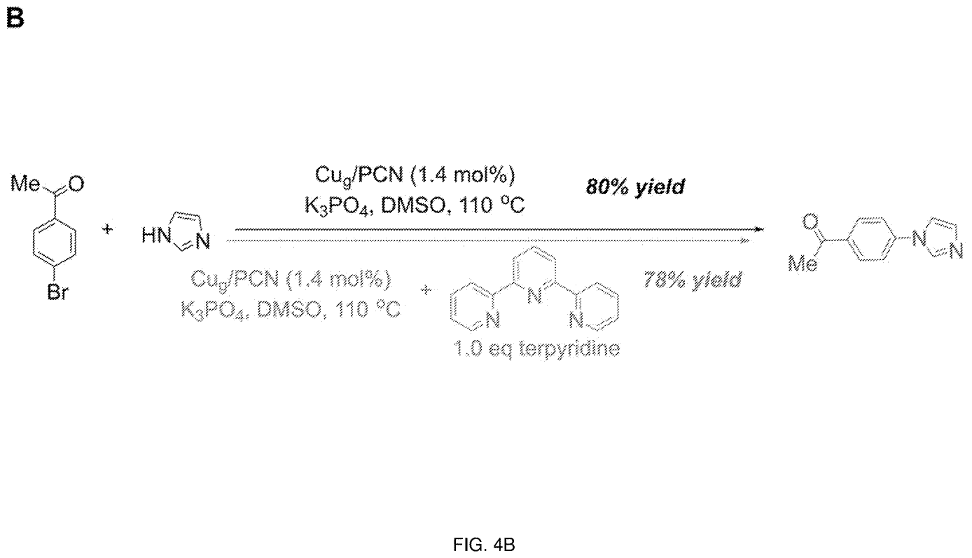

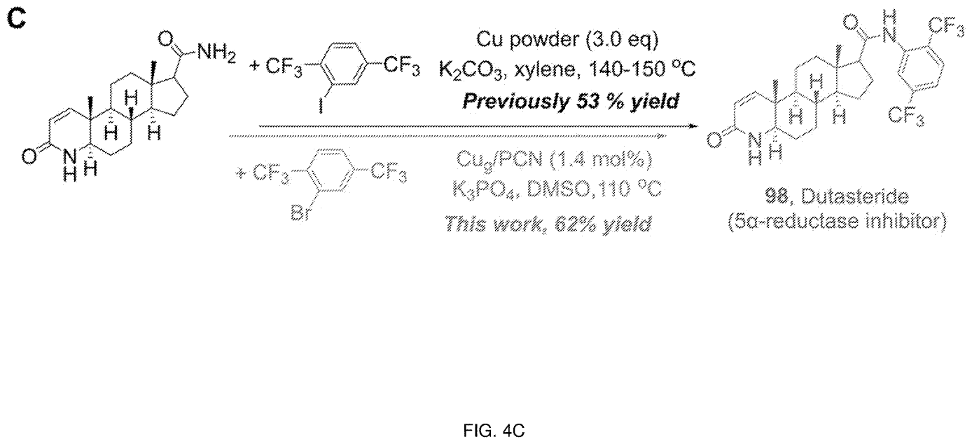

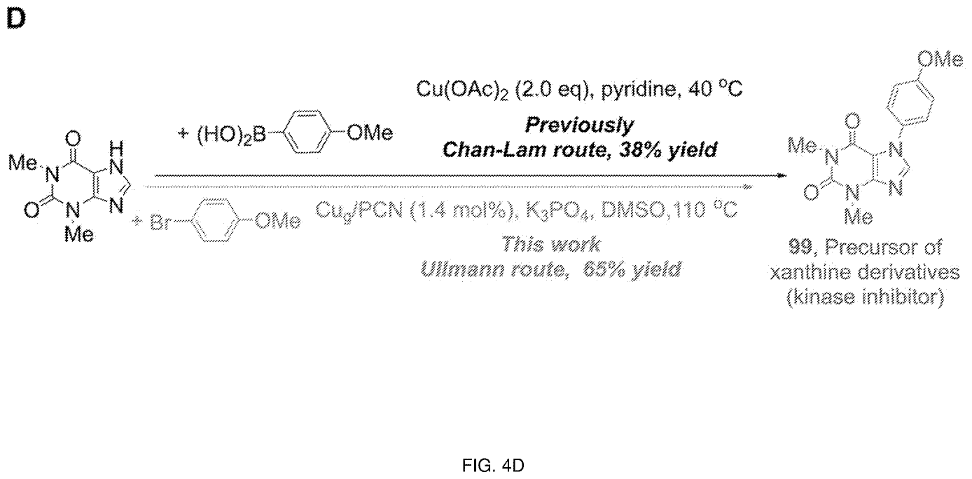

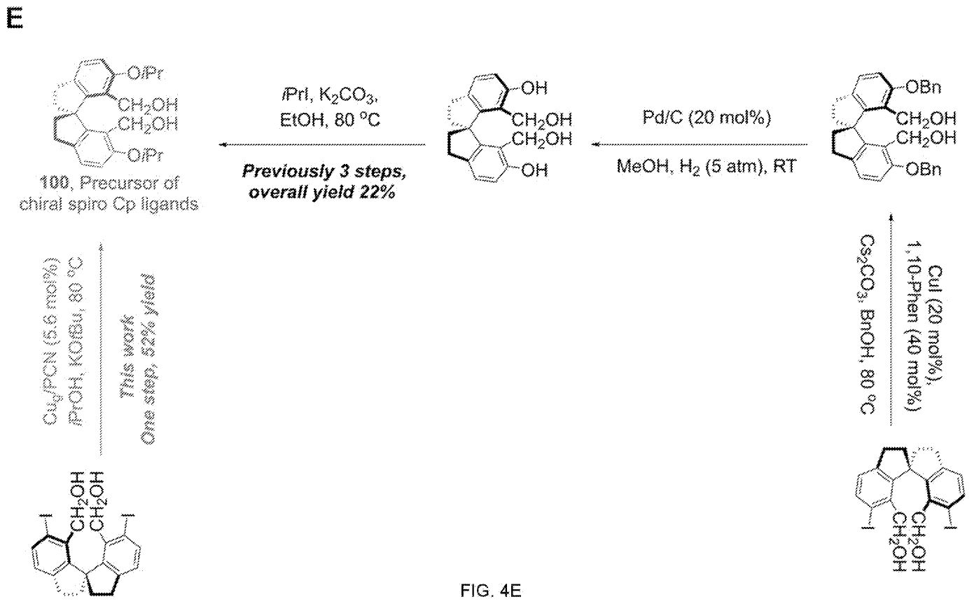

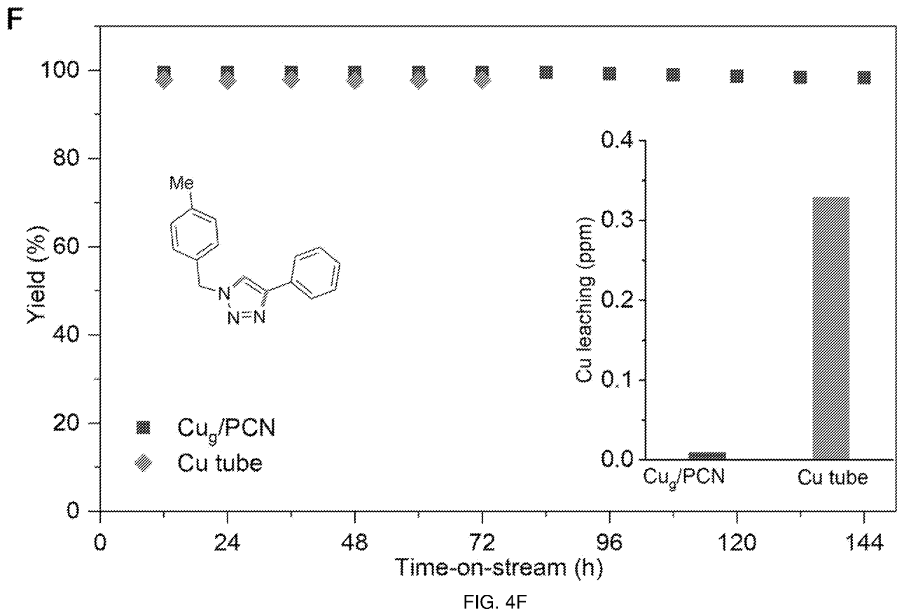

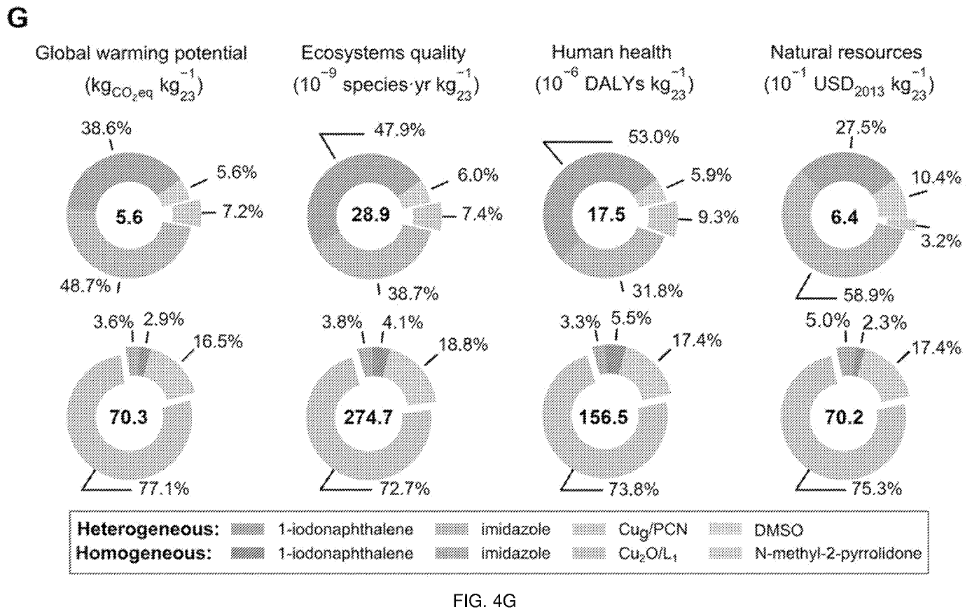

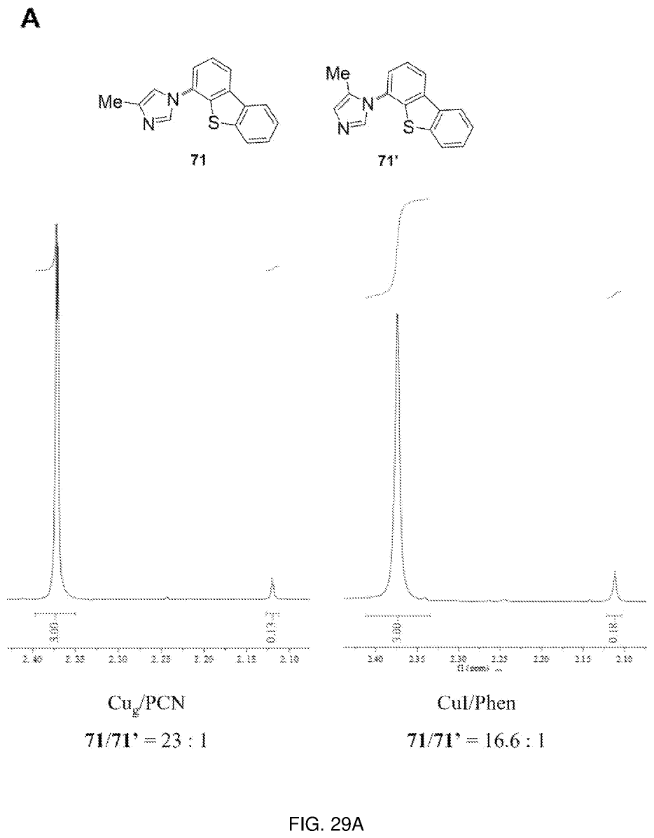

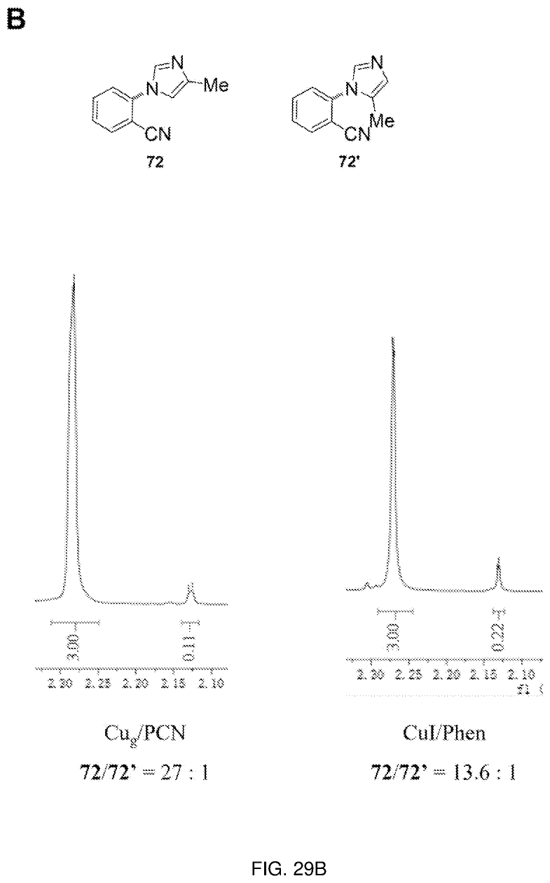

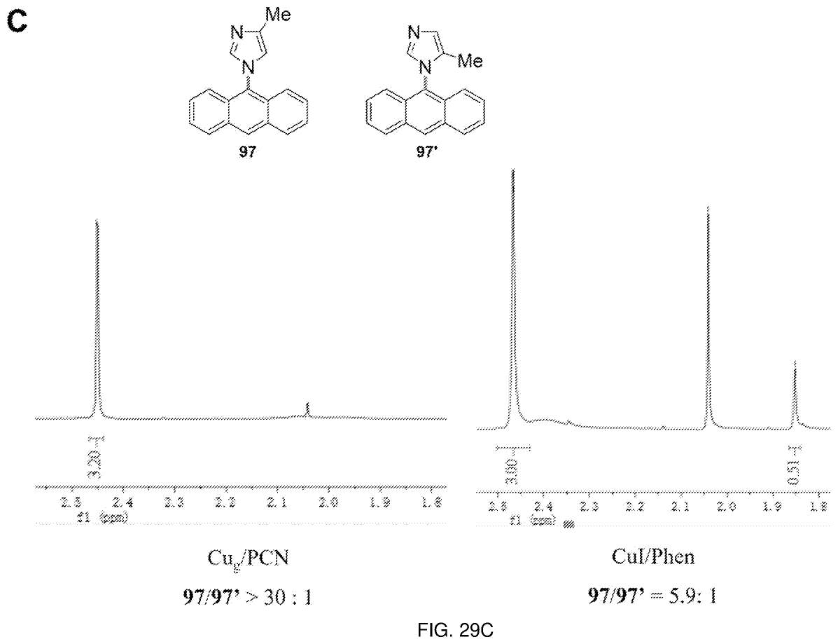

The versatility of Cu_{g}/PCN was further showcased by evaluating the catalyst in various synthetic scenarios (FIGS. 4A, 4B, 4C, 4D and 4E). In the N-arylation of imidazoles (97, 71, 72), Cu_{g}/PCN exhibited enhanced regioselectivity compared to the benchmark homogeneous 1,10-phenanthroline-ligated copper catalyst, suggesting a major advantage of GACs in facilitating the formation of multifunctional heterocycles that are common in natural products and pharmaceuticals (FIG. 4A, FIGS. 8A and 8B and FIGS. 29A, 29B and 29C). Promisingly, the performance of Cu_{g}/PCN in the C—N coupling reaction was not compromised by an exogenous equivalent of terpyridine (FIG. 4B), highlighting the catalyst's tolerance to multi-coordinating entities. Moreover, the Cu_{g}/PCN catalyst addressed practical shortcomings of reported synthetic routes to synthetically challenging pharmaceuticals. For example, a more readily available bromide substrate (this work, 1.4 mol % Cu catalyst, 62% yield) was successfully employed in C—N coupling (previously iodide substrate, 3 equivalents Cu powder, 53% yield), improving the overall synthesis of Dutasteride (98), a synthetic 4-azasteroid derivative used primarily to treat prostate disease (FIG. 4C). Similarly, a more efficient and cost-effective Ullmann coupling (this work, 1.4 mol % Cu catalyst, 65% yield) using a bromide substrate was designed as an alternative for the previously reported Chan-Lam coupling involving a more expensive boronic acid (2 equivalents Cu complex, 38% yield) for the synthesis of a precursor of xanthine derivatives (99), which are used as kinase inhibitors (FIG. 4D). By using catalytic amounts of recyclable Cu_{g}/PCN, stoichiometric amounts of copper waste can be avoided, which will otherwise lead to product contamination and other environmental issues. Additionally, Cu_{g}/PCN demonstrated its capability in promoting cross-coupling reactions involving sterically demanding substrates, enabling the synthesis of the precursor of chiral spiro Cp ligands (100), which are important in asymmetric catalysis. Through this direct cross-coupling with Cu_{g}/PCN, the precursor can be secured in a single step in 52% yield, compared to the previous route that required 3 steps to deliver 100 in 22% overall yield because direct cross-coupling with isopropanol was inefficient in the presence of the homogeneous copper-based catalyst (FIG. 4E). These examples underscore the power of Cu_{g}/PCN in overcoming challenges in organic synthesis, rendering them attractive candidates for practical adoption in fine chemical production.

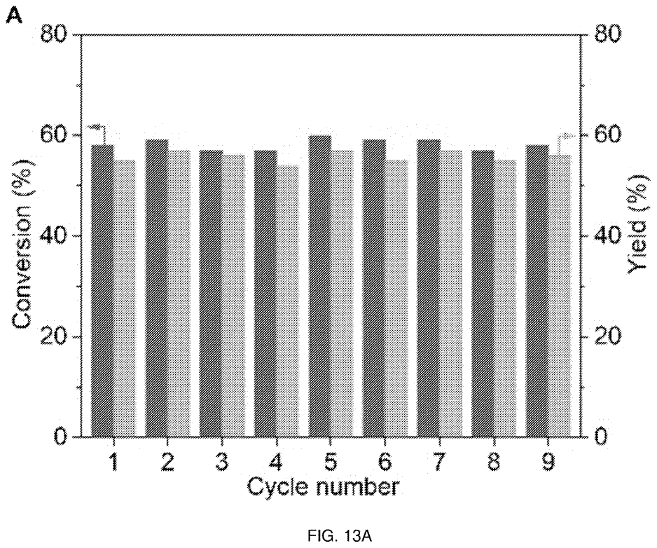

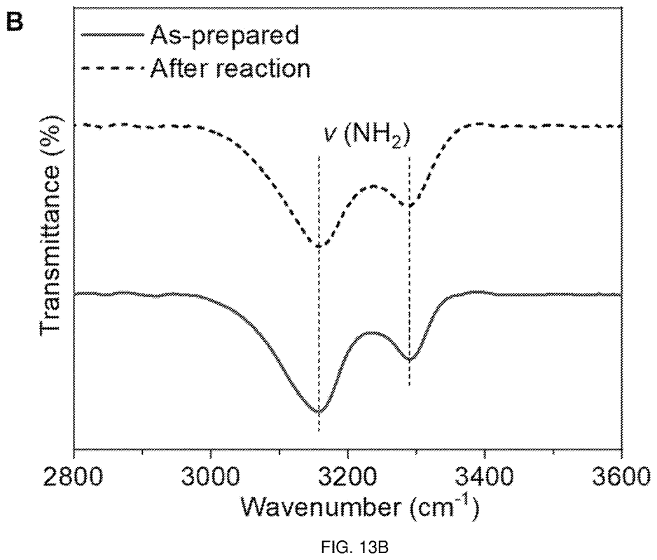

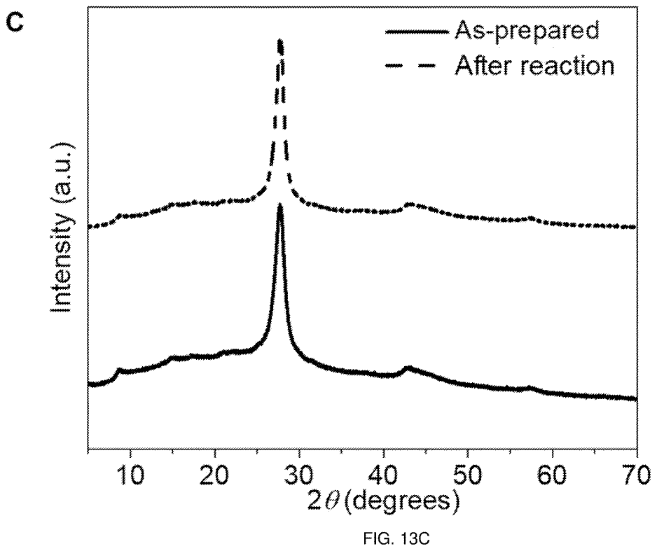

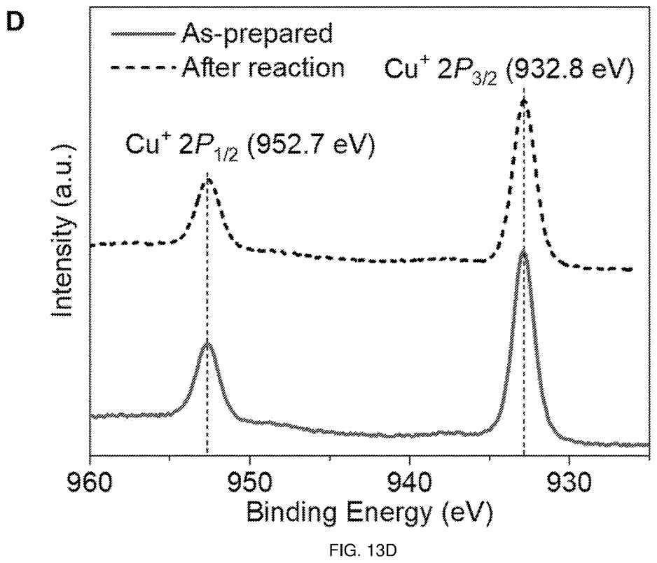

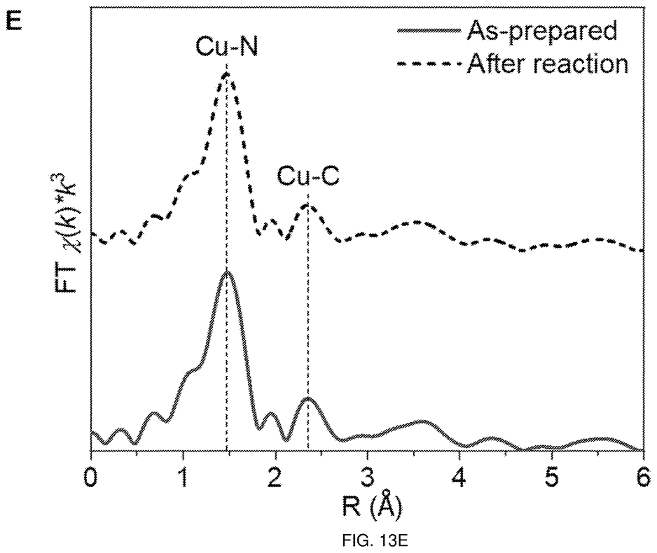

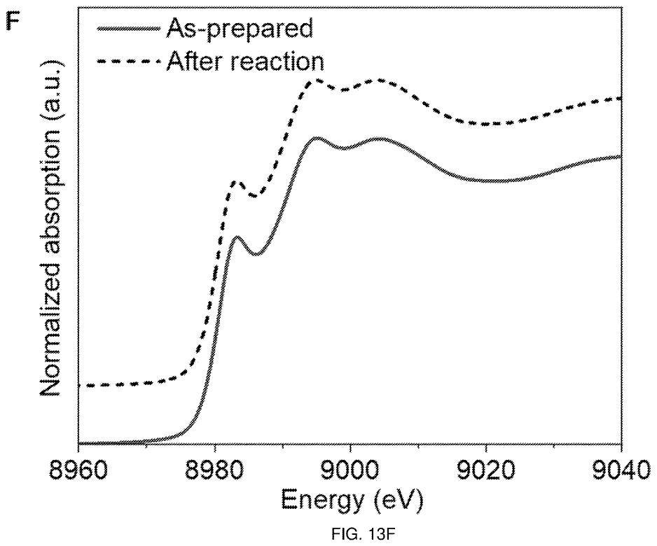

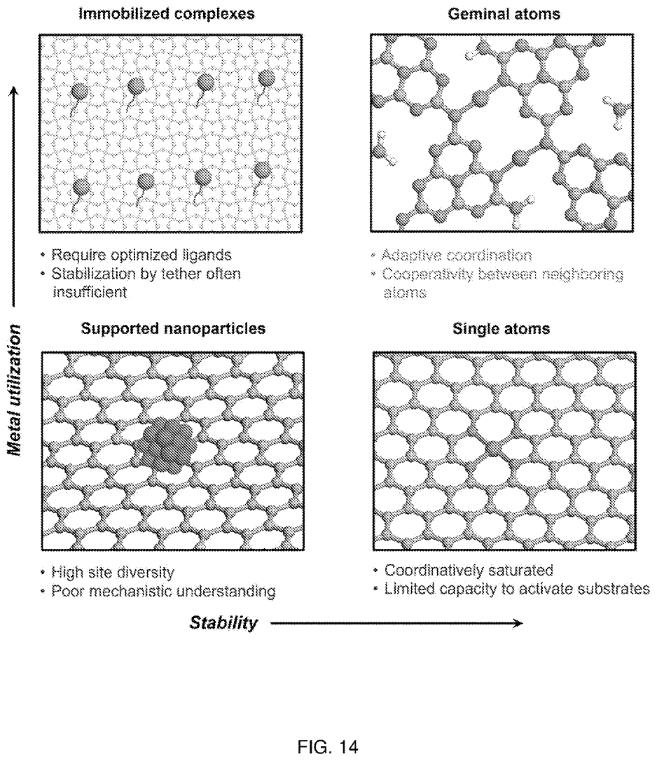

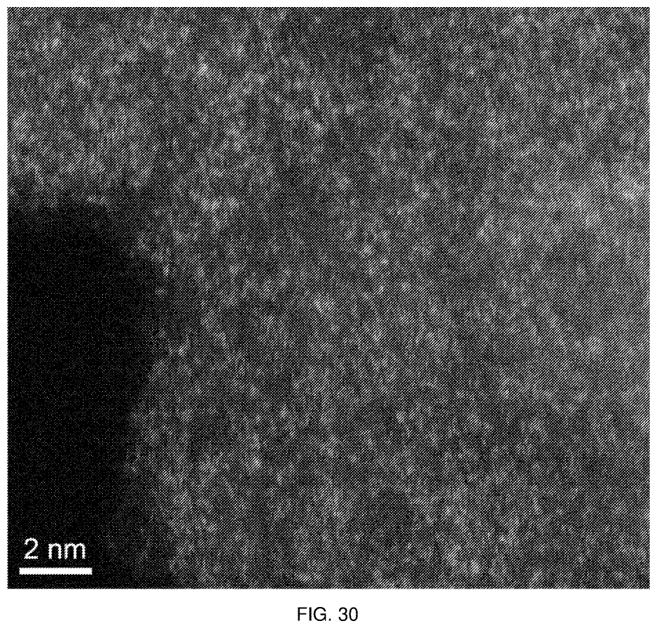

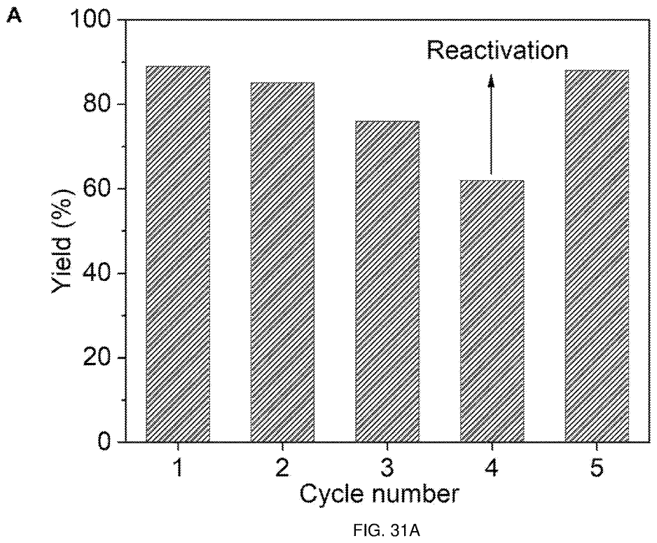

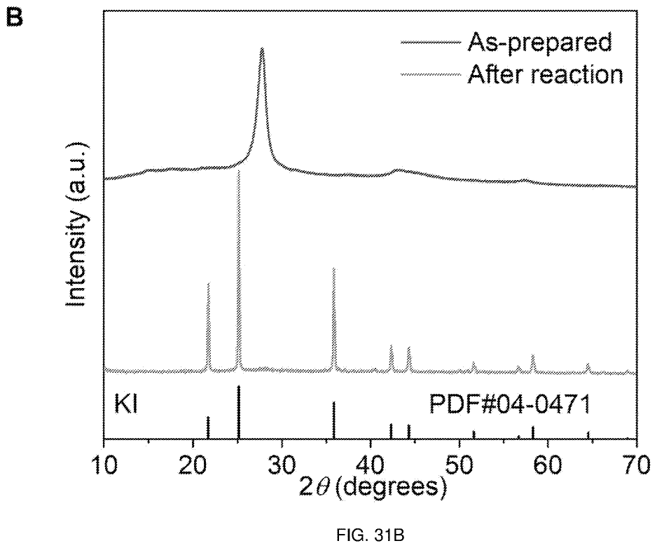

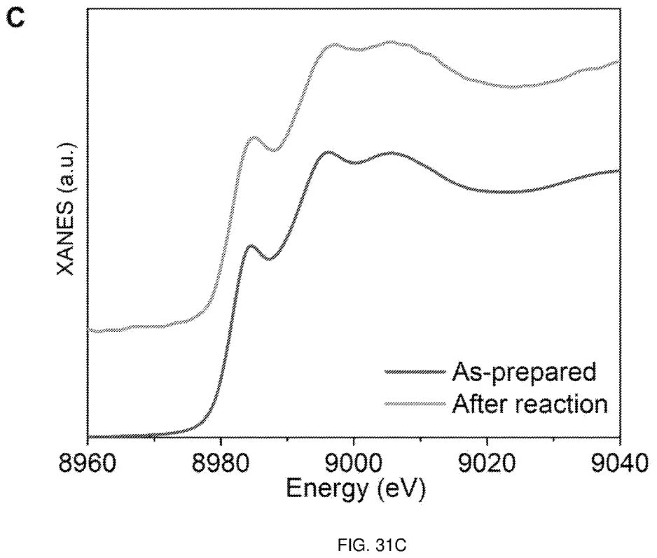

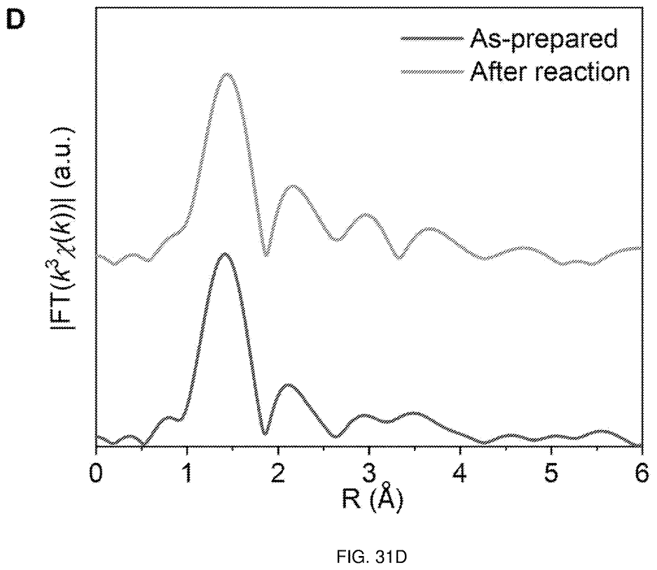

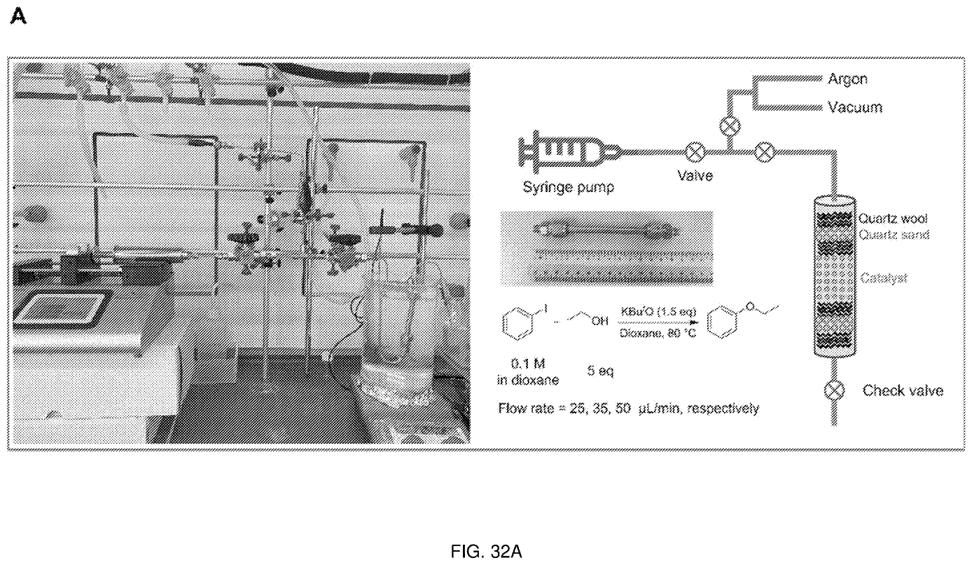

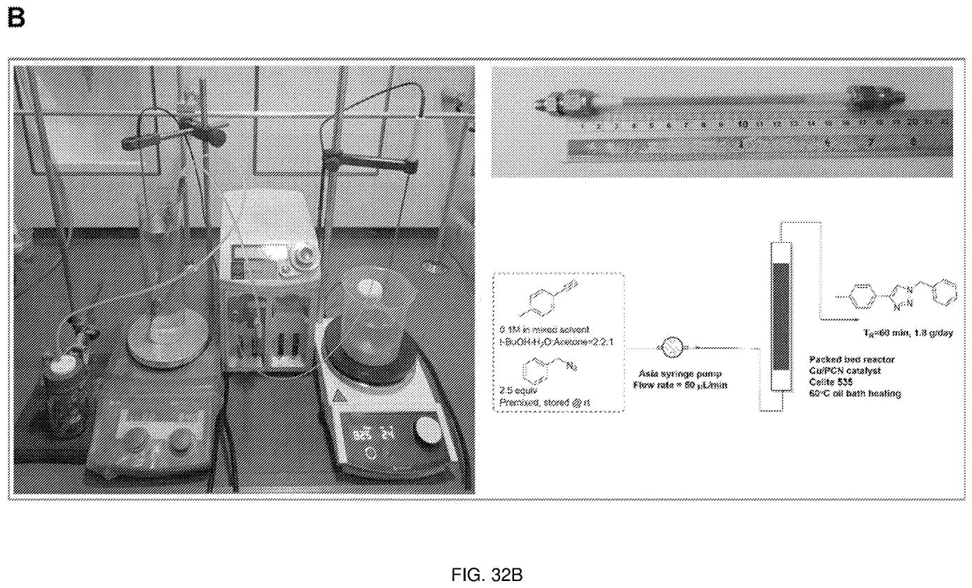

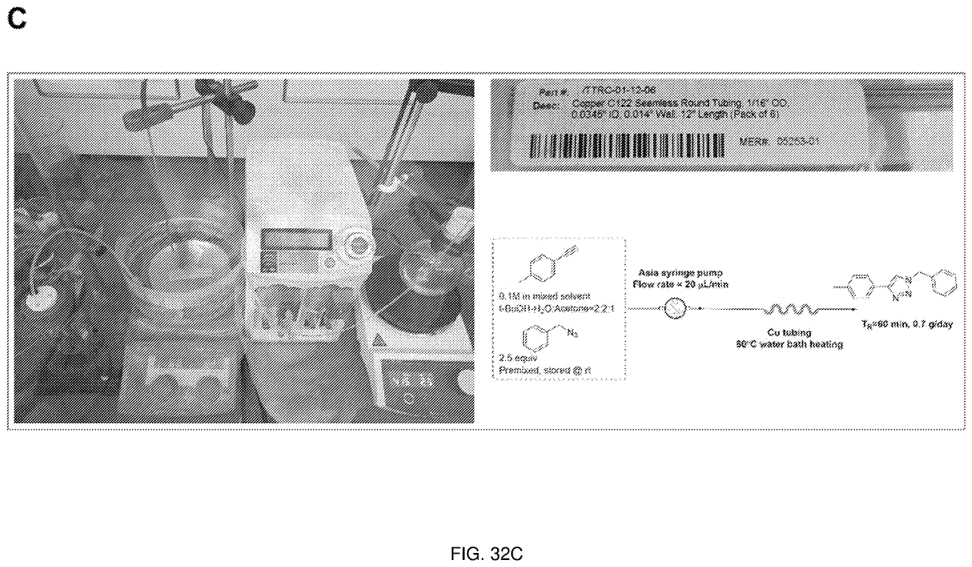

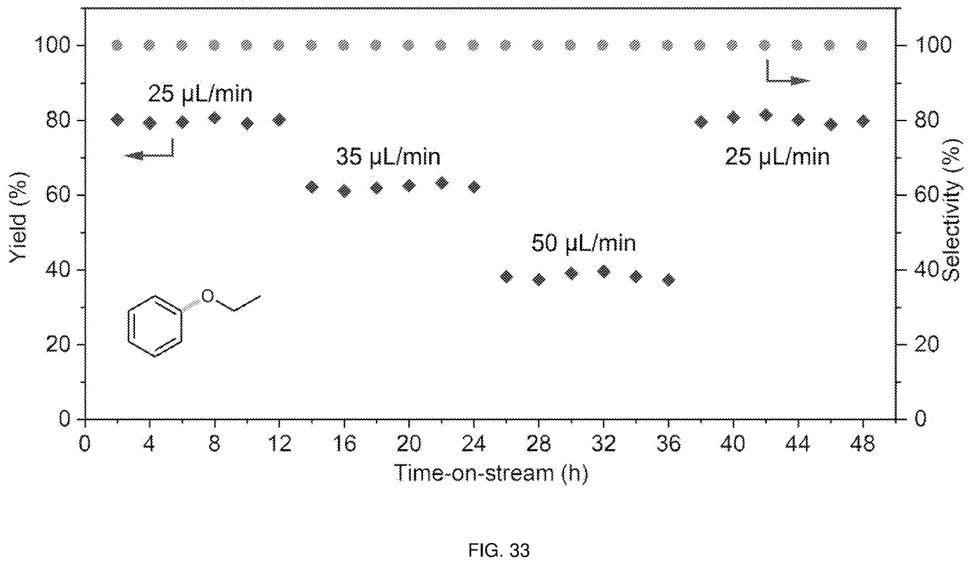

A key additional advantage of using heterogeneous catalysts lies in their recoverability and reusability. Cu_{g}/PCN demonstrated excellent durability and recyclability in the C—O coupling reaction as shown in FIGS. 13A, 13B, 13C, 13D, 13E and 13F, with no appreciable conversion and selectivity decrease over nine consecutive runs. Consistently, ICP-AES analysis of the used catalyst confirms the virtually identical amounts of Cu to the fresh Cu_{g}/PCN, accompanied by the absence of any detectable Cu ions in solution. The ADF-STEM image acquired over the used Cu_{g}/PCN catalyst showed a high density of atoms with no visible nanoparticle aggregation, suggesting that the atomic dispersion of copper species was preserved after the catalytic reaction (FIG. 30). FTIR, XRD, XPS, Cu K-edge XANES and EXAFS analysis also revealed the identical atomic structures of Cu in the used catalyst to the fresh Cu_{g}/PCN (FIGS. 13A, 13B, 13C, 13D, 13E and 13F). In addition, the coupling of 4-iodotoluene with ethanol occurred in the presence of 0.01 mol % Cu (Cu_{g}/PCN) to give the desired product in 80% yield, corresponding to a turnover number (TON) of 8000 that is almost two orders of magnitude higher than the homogeneous copper-catalyzed cross-coupling of an aryl halide with aliphatic alcohol. It is worth noting that the obtained TON value is likely underestimated due to the active site blockage by the deposition of insoluble salts (FIGS. 31A, 31B, 31C and 31D). Furthermore, motivated by major developments to improve synthetic efficiency and the intensification of chemical processes, the study translated the batch synthesis over Cu_{g}/PCN to a continuous flow protocol, which has distinct advantages in terms of automation and process optimization (FIGS. 32A, 32B and 32C). Evaluation in a custom-made packed-bed reactor evidenced a stable gradient production of ethoxybenzene under varying flow rates (FIG. 33) and a constant production of 1-(4-methylbenzyl)-4-phenyl-1H-1,2,3-triazole on stream for more than 144 h with no obvious variation in selectivity or yield (FIG. 4F), as well as the negligible leaching of Cu ions into solution compared to commercially available copper tube flow reactors (inset of FIG. 4F).

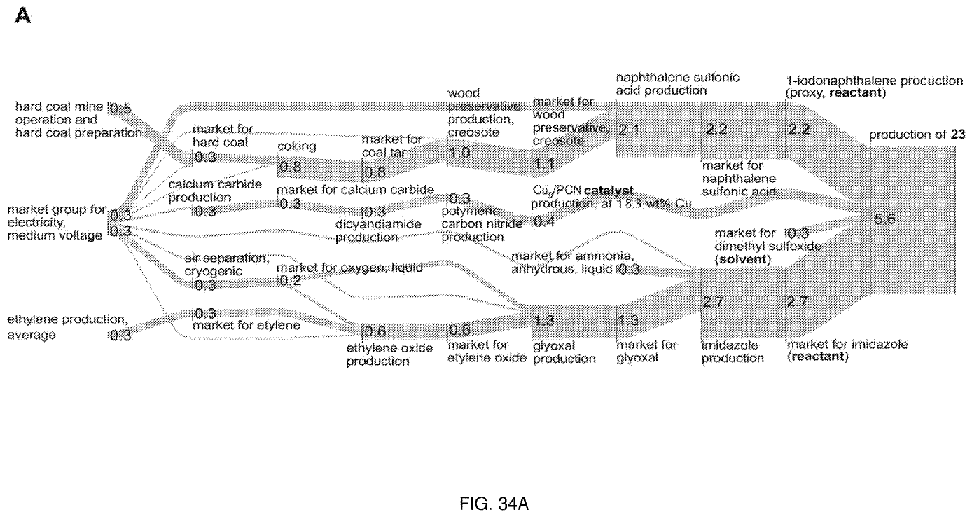

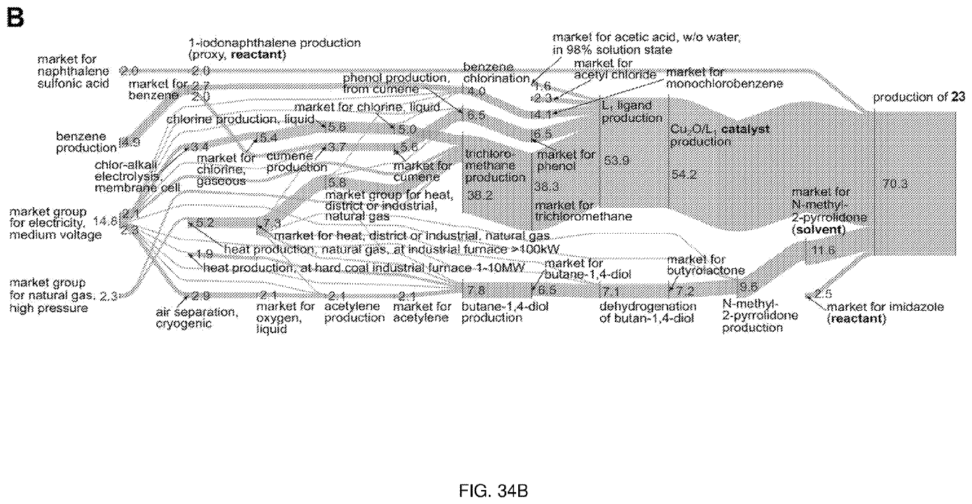

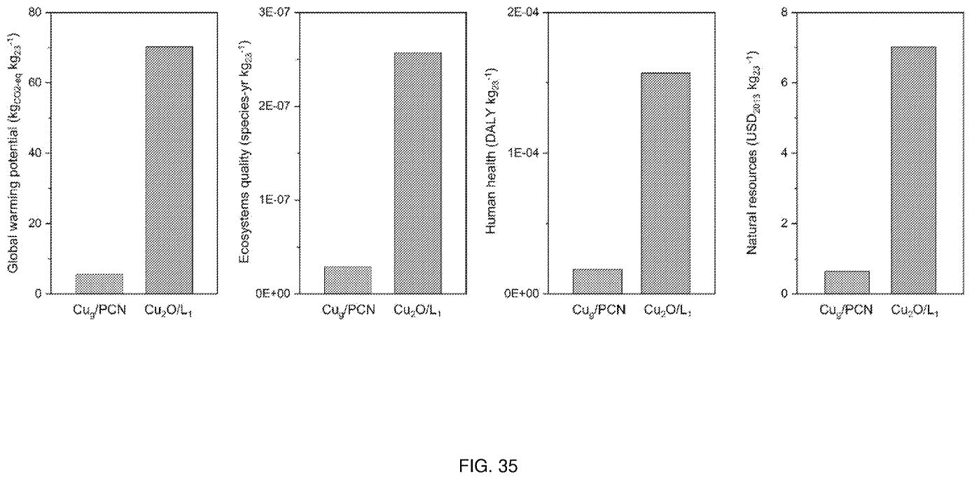

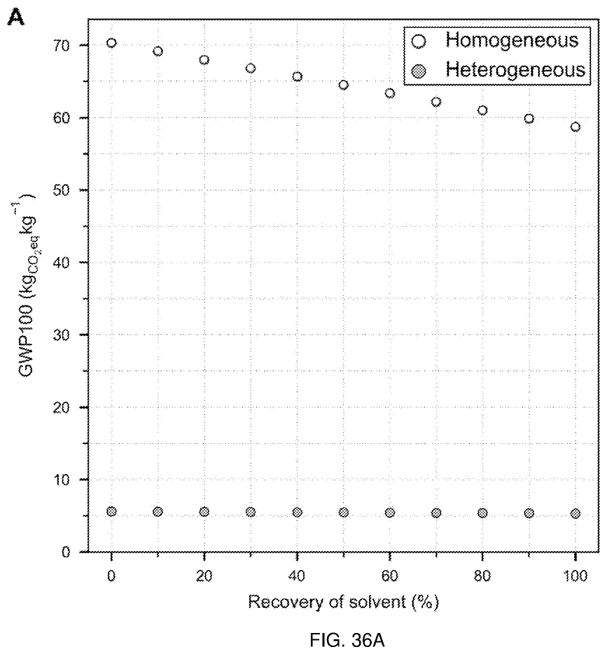

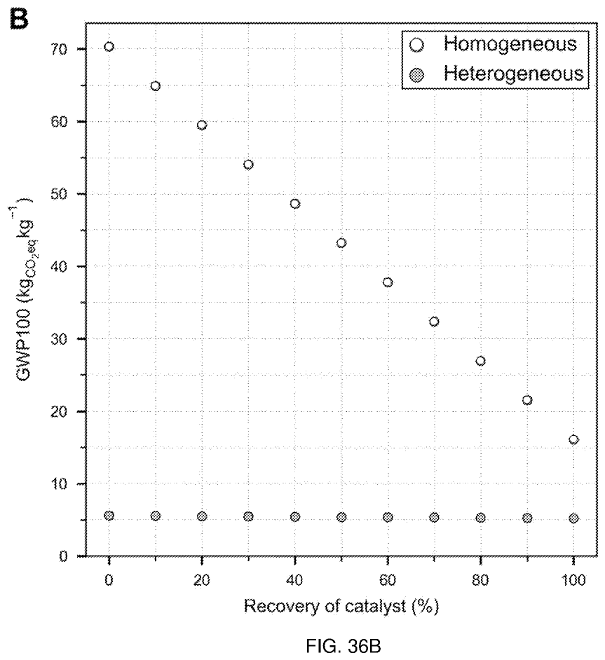

Finally, the study has quantified the environmental benefits of the geminal-atom catalyzed route compared to conventional homogeneous synthesis through LCA. This analysis, which provided a detailed cradle-to-gate evaluation of the impacts of all the reaction components, revealed substantially reduced environmental footprint of the heterogeneous catalyst and highlighted the dominant contribution of the ligand in the homogeneous system (FIG. 4G and FIG. 34A, 34B and FIG. 35). Moreover, sensitivity analysis on the LCA results shows that it would be difficult to find a homogenous counterpart that displays significantly better environmental performance, since contributions of the catalyst and solvent to the total impact in the heterogeneous system is already low (FIGS. 36A and 36B).

### Example 6: Summary

In summary, the design of geminal metal single atoms catalysts anchored on polymeric carbon nitride has been presented above, demonstrating their promising potential in heterogeneously-catalyzed cross-coupling reactions. The specific proximity of two paired, low-valent and electronically-isolated geminal metal centers permit a cooperative bridge cross-coupling mechanism leading to greatly enhanced efficiency compared to conventional monomeric metal sites in single-atom catalysts. This catalytic platform enables the concise and cost-effective construction of complex pharmaceutical compounds, comparing favourably with state-of-the-art homogeneous catalytic systems in terms of applicability and synthetic scope. Beyond solving the long-standing oxidative addition challenge of copper-based catalysts, the concept of geminal atom catalysis with dynamic active sites provides a generalizable framework to access myriad catalysts by adjusting the type and combination of the geminal metal centers to suit different synthetic demands.

### Example 7: Methods

### 7.1. Synthesis of Polymeric Carbon Nitride (PCN)

Bulk PCN was prepared by calcining dicyandiamide at 550 °C (2.3 °C min⁻¹ heating rate) in a crucible for 3 h in static air. Exfoliated PCN was obtained via the thermal exfoliation at 500 °C (5 °C min⁻¹ heating rate) for 5 h in static air. The exfoliation procedure can recycle several times until the PCN been exfoliated efficiently.

### 7.2. Synthesis of Cu_{g}/PCN

CuCl₂ (0.64 g) and exfoliated PCN (0.70 g) were dispersed in 200 ml formamide, sonicated for 10 min then stirred in an oil bath (120 °C) for 12 h, following centrifugation and washing thoroughly by ethanol several times. The oven-dried powder (80 °C) was subsequently heated to 500 °C with a heating rate of 2 °C min⁻¹ and kept for 5 h with the protection of N₂ flow. Cu₁/PCN with the Cu contents of 0.2, 0.9, 1.8, 8.0, and 10.8 wt. % were prepared using 0.2, 1.0, 2.0, 10.0, and 15.0 wt. % Cu feeding, respectively.

### 7.3. Material Characterization

Wide-angle X-ray diffraction (XRD) patterns were collected on a Bruker D8 Focus Powder X-ray diffractometer using Cu Ka radiation (40 kV, 40 mA) at room temperature. ADF-STEM imaging was carried out in an aberration-corrected JEOL ARM-200F system equipped with a cold field emission gun operating at 60 kV. X-ray photoelectron spectroscopy (XPS) measurements were carried out in a custom-designed ultrahigh-vacuum system with a base pressure lower than 2×10⁻¹⁰ mbar. Al Ka (hn=1486.7 eV) was used as the excitation source for XPS. AFM image was acquired on a Bruker FastScan AFM using Tapping mode. The metal content in all the catalysts was measured by an inductively coupled plasma atomic emission spectrometer (ICP-AES). FTIR spectra were performed at 25 °C on a Bruker Equinox 55 spectrometer equipped with a mercury cadmium telluride detector. X-band EPR spectra were obtained with a JEOL (FA200) spectrometer. ¹H, ¹³C and ¹⁹F NMR spectra were recorded on Bruker Avance Neo 400 or 500 spectrometers. The STM experiments were conducted in UHV conditions (base pressure, <2×10⁻⁹ mbar) at 4.4 K using a Scienta Omicron LT-STM system. The X-ray absorption near-edge structure (XANES) and the extended X-ray absorption fine structure (EXAFS) measurements were carried out at the XAFCA beamline of the Singapore Synchrotron Light Source (SSLS) according to Du, Y. et al. XAFCA: a new XAFS beamline for catalysis research. J. Synchrotron Radiat. 22, 839-843 (2015), the contents of which are fully incorporated herein by reference. A Si (111) double crystal monochromator was used to filter the X-ray beam. High resolution XANES was performed using the third harmonic of the Si (111) double crystal monochromator. Copper foil was used for the energy calibration, and all samples were measured under transmission mode. EXAFS oscillations X(k) were extracted and analysed using the Demeter software package according to Ravel, B. & Newville, M. ATHENA, ARTEMIS, HEPHAESTUS: data analysis for X-ray absorption spectroscopy using IFEFFIT. J. Synchrotron Radiat. 12, 537-541 (2005), the contents of which are fully incorporated herein by reference. For cross-coupling reactions, all the reactants and catalysts were added in a nitrogen-filled glovebox to avoid potential performance losses due to substrate instability in the presence of oxygen or, in particular, trace amounts of water.

### 7.4. C—N Bond Formation

An oven-dried screw-top reaction tube was equipped with a stir bar. Aryl halide (0.2 mmol), nitrogen-based nucleophile (0.24 mmol for N-heterocyclic compounds, 0.3 mmol for primary and secondary amines), Cu_{g}/PCN (1.0 mg, 1.4 mol % Cu), 0.4 mmol base (K₃PO₄ for N-heterocyclic compounds, NaOH for primary and secondary amines) and anhydrous DMSO (1.0 mL) were sequentially added. The reaction tube was sealed with a Teflon-lined screw cap, removed from the glovebox, placed in an oil bath preheated to 110 °C and stirred for 28 h. After cooling to rt, the reaction cap was removed, and the reaction mixture was concentrated in vacuo with the aid of a rotary evaporator. The resulting residue was then purified by silica gel column chromatography to give the pure product.

### 7.5. C—O Bond Formation

An oven-dried screw-top reaction tube was equipped with a stir bar. Aryl iodide (0.2 mmol), alcohol (0.4 mmol, for di-halide aromatic ring, 0.8 mmol), Cu_{g}/PCN (1.0 mg, 1.4 mol % Cu), KOtBu (0.3 mmol, for di-halide aromatic ring, 0.6 mmol) and anhydrous dioxane (2.0 mL) were sequentially added. The reaction tube was sealed with a Teflon-lined screw cap, removed from the glovebox, placed in an oil bath preheated to 80 °C and stirred for 18 h. After cooling to room temperature (rt), the reaction cap was removed, and the reaction mixture was concentrated in vacuo with the aid of a rotary evaporator. The resulting residue was then purified by silica gel column chromatography to give the pure product.

### 7.6. C—C Bond Formation

An oven-dried screw-top reaction tube was equipped with a stir bar. Aryl halide (0.2 mmol), alkyne (0.3 mmol, for di-halide aromatic ring, 0.6 mmol), Cu_{g}/PCN (1.0 mg, 1.4 mol % Cu), Cs(OH)₂·H₂O (0.3 mmol, for di-halide aromatic ring, 0.6 mmol) and anhydrous DMSO (1.0 mL) were sequentially added. The reaction tube was sealed with a Teflon-lined screw cap, removed from the glovebox, placed in an oil bath preheated to 110 °C and stirred for 28 h. After cooling to rt, the reaction cap was removed, and the reaction mixture was concentrated in vacuo with the aid of a rotary evaporator. The resulting residue was then purified by silica gel column chromatography to give the pure product.

### 7.7. C—S Bond Formation

An oven-dried screw-top reaction tube was equipped with a stir bar. Aryl halide (0.2 mmol), mercaptan/thiophenol (0.3 mmol), Cu_{g}/PCN (1.0 mg, 1.4 mol % Cu), NaOtBu (0.30 mmol) and anhydrous dioxane (2.0 mL) were sequentially added. The reaction tube was sealed with a Teflon-lined screw cap, removed from the glovebox, placed in an oil bath preheated to 80 °C and stirred for 18 h. After cooling to rt, the reaction cap was removed, and the reaction mixture was concentrated in vacuo with the aid of a rotary evaporator. The resulting residue was then purified by silica gel column chromatography to give the pure product.

### 7.8. Azide-Alkyne Cycloaddition

Azide-alkyne cycloaddition was carried out in a 10 mL glass tube with Cu_{g}/PCN catalyst (1 mg, 1.4 mol % Cu), 0.2 mmol of aryl acetylene, 0.6 mmol (1.2 mmol for di-aryl acetylene) of benzyl azide, decane (0.2 mmol) and 3 mL of a 1:1 water/acetonitrile mixture and stirred at 60 °C for 24 h. After cooling to rt, the mixture was diluted with water and extracted with DCM. The crude product was analyzed by gas chromatography-mass spectrometry (GC-MS) for determination of the yield.

### 7.9. Recycling Test

4-iodotoluene (20.0 mmol, 4360 mg), ethanol (40.0 mmol, 1840 mg), Cu_{g}/PCN (100 mg, 1.4 mol % Cu), KOtBu (30 mmol, 3368 mg), decane (10.0 mmol, 1950 μL) and anhydrous dioxane (200 mL) were sequentially added to an oven-dried screw-top reaction bottle equipped with a stir bar. The reaction bottle was sealed with a Teflon-lined screw cap, removed from the glovebox, placed in an oil bath preheated to 80 °C and stirred for 8 h. After cooling to rt, the bottle was allowed to stand for 1 h. The reaction cap was then removed, an aliquot of the solution transferred into a vial, diluted with EtOAc, and analysed by GC to determine the yield. The remaining reaction mixture was removed by vacuum filtration using Nylon filter membranes. Prior to use in the next cycle reaction, the catalyst was washed in dioxane, EtOH and DI water three times, and dried overnight at 80 °C.

### 7.10. Continuous Flow Synthesis

The continuous-flow reactions were conducted in a tubular reactor. 250 mg of Cu_{g}/PCN (40-100 mesh particles) and 250 mg quartz sand/celite at each end was packed in steel/quartz column reactor and the mixture of reactants, bases and solvents was pumped through the column at a certain flow rate. The real-time yield of product was detected by GC-MS.

### 7.11. In-Situ X-Ray Absorption Spectroscopy

In a nitrogen-filled glovebox, an oven-dried screw-top reaction tube was equipped with a stir bar. 4-iodobenzene (1.0 mmol), ethanol (2.0 mmol), Cu_{g}/PCN (5.0 mg), KOtBu (1.5 mmol) and anhydrous dioxane (5.0 mL) were sequentially added. The reaction tube was sealed with a Teflon-lined screw cap, removed from the glovebox, placed in an oil bath preheated to 80 °C and stirred for 3 h. After that, the mixture was quickly transferred to a plastic bag for XAFS measurement.

### 7.12. In-Situ Electron Paramagnetic Resonance Spectroscopy

In a nitrogen-filled glovebox, an oven-dried quartz EPR tube was equipped with a stir bar. 4-iodobenzene (0.05 mmol), ethanol (0.1 mmol), Cu_{g}/PCN (4.0 mg), KOtBu (0.075 mmol) and anhydrous dioxane (0.5 mL) were sequentially added. The reaction tube was sealed with a screw cap, removed from the glovebox, placed in an oil bath preheated to 80 °C under stirring. For each indicated time, the EPR tube was transferred from the oil bath to the EPR spectrometer and take the corresponding measurement. After collecting the data, the EPR tube was retransferred to the oil bath for continuing reactions.

### 7.13. Computational Details

The quantum-chemical studies were carried out at the level of Kohn-Sham density functional theory (DFT) using two types of theoretical formalisms, the periodic DFT approach and the molecular DFT approach for a selected cluster model. These two approaches provide a more comprehensive computational data for understanding the geometric structures, electronic structures, chemical bonding, and catalytic mechanism. The periodic DFT calculations were performed with Vienna Ab-initio Simulation Package (VASP) according to Kresse, G. & Furthmüller, J. Efficient iterative schemes for ab initio total-energy calculations using a plane-wave basis set. Phys. Rev. B 54, 11169 (1996), the contents of which are fully incorporated herein by reference. These first-principles calculations under spin-polarized Kohn-Sham formulism were carried out for the structure optimizations, electronic structure analysis and transition state search with CI-NEB (climbing image nudged elastic band) according to Henkelman, G., Uberuaga, B. P. & Jónsson, H. A climbing image nudged elastic band method for finding saddle points and minimum energy paths. J. Chem. Phys. 113, 9901-9904 (2000), the contents of which are fully incorporated herein by reference. The generalized gradient approximation (GGA) in Perdew-Burke-Ernzerhof (PBE) exchange-correlation functional according to Perdew, J. P., Burke, K. & Ernzerhof, M. Generalized gradient approximation made simple. Phys. Rev. Lett. 77, 3865 (1996), the contents of which are fully incorporated herein by reference, the projector-augmented wave (PAW) method according to Kresse, G. & Joubert, D. From ultrasoft pseudopotentials to the projector augmented-wave method. Phys. Rev. B 59, 1758 (1999), the contents of which are fully incorporated herein by reference and a plane wave basis set with the cut-off energy of 450 eV were employed in all the calculations. The convergence criterion for structural relaxations and CI-NEB was set to 0.01 eV Å⁻¹ and 0.05 eV Å⁻¹, respectively. A vacuum layer of 15 Å in the direction perpendicular to PCN film was included to avoid artificial interaction between the neighbouring images. 5×5×1−k point sampling was applied in all calculations. A 2×2 supercell of PCN structure was adopted to model the Cu doped PCN 2D film. The supercell used in the calculation is shown in FIG. 20A and FIG. 20B. The adsorption energy is defined as E_{ads}=E(adsorbed)−E(separated), which means stronger adsorption with more negative adsorption energy. The calculated charge density distributions (CDD) and projected density of states (PDOS) of the single-atom Cu and two-atom Cu (geminal) doped carbon-nitrogen 2D material are shown in FIGS. 11A, 11B, 11C and 11D.

The molecular DFT calculations were carried out on the cluster model constructed to compare with the periodic DFT calculations, as shown in FIG. 20B. Spin-restricted and spin-unrestricted (polarized) Kohn-Sham calculations (RKS and UKS) were respectively carried out for closed-shell singlet states and open-shell singlet/triplet states using Gaussian-16 suite of programs according to Frisch, M. et al. (Gaussian, Inc. Wallingford, CT, 2016), the contents of which are fully incorporated herein by reference. The polarized split-valence 6-31G(d) Gaussian basis sets were used for the C, N, O, and H atoms according to Francl, M. M. et al. Self-consistent molecular orbital methods. XXIII. A polarization-type basis set for second-row elements. J. Chem. Phys. 77, 3654-3665 (1982), the contents of which are fully incorporated herein by reference. Effective core potential (ECP) of LANL2DZ according to Hay, P. J. & Wadt, W. R. Ab initio effective core potentials for molecular calculations. Potentials for K to Au including the outermost core orbitals. J. Chem. Phys. 82, 299-310 (1985), the contents of which are fully incorporated herein by reference was adopted for Cu, together with the LANL2DZ basis set for the valence electrons of Cu [Ne]3s²3p⁶3d⁶4s². Harmonic vibrational frequencies were obtained at the same levels in this work to check the properties of the stationary points. All of the structures discussed in the present work are minimal or transition states on the corresponding potential energy surfaces, as confirmed by zero or one imaginary frequency, respectively. To further refine the electronic energies, the single-point calculations on the optimized geometries were also performed using the hybrid B3LYP functional when specified according to Becke, A. D. Density-functional exchange-energy approximation with correct asymptotic behavior. Physical review A 38, 3098 (1988); and Lee, C., Yang, W. & Parr, R. G. Development of the Colle-Salvetti correlation-energy formula into a functional of the electron density. Phys. Rev. B37, 785 (1988), the contents of which are fully incorporated herein by reference. The study also plotted the radial distribution probability density (FIG. 22), D(r)=r²R(r)², of Cu(II) atomic orbitals (AOs) to elucidate the chemical bonding interaction of geminal Cu . . . Cu when the distance is changed.

The adsorption energy (E_{ads}) for molecule (M) on the GACs was defined as

E_{ads}=E_{M-GAC}−E_{GAC}−E_{M}

where E_{M-GAC}, E_{GAC}, and E_{M} are the total energy for GACs with the M-molecule adsorbed, the GACs, and the absorption molecule, respectively.

In order to explore the catalytic reaction mechanism of various chemical reactions on GACs, the study has chosen to focus on C—O coupling reactions. The reaction between C₆H₅I and CH₃OH was selected as an example and the energy profiles calculated with PBE functional for the various pathways and processes are shown in FIG. 21A, 21B, 21C, FIG. 22, FIG. 23, FIG. 24, and FIG. 25. Both the direct dehalogenation and dehydrogenation of C₆H₅I and CH₃OH and the indirect ones with the help of strong base of KOtBu were studied. Two reaction pathways (indirect and direct pathways) for C—O coupling between *C₆H₅ and CH₃O* were also explored. Through the indirect pathway, C—O coupling of CH₃O* and *C₆H₅ to form C₆H₅OCH₃ is much more difficult than in the direct pathway in which C—O coupling facilitated by dynamic formation of a direct Cu—Cu 4s-4s bond. The later represents a new reaction mechanism for C—O coupling via disproportional process of copper oxidation state from Cu(II)—Cu(II) to Cu(Ill)-Cu(I). The atomic numbering of the Cu_{A} . . . Cu_{B} active sites together with the N-atoms of GACs as well as the calculated bond distances and Wiberg bond orders for intermediate states IS5 and IS6 are listed in Table 5. The Mulliken spin density populations of the atoms in the open-shell intermediates ³IS3 and ³IS4 are listed in Table 7 to facilitate the assignment of the oxidation states of Cu_{A} . . . Cu_{B} active sites in the disproportional process. Here, the superscripts 1 and 3 (e.g., in ³IS3 and ³IS4) indicate the spin multiplicity 2S+1 of the system.

### 7.14. Life Cycle Assessment

Life cycle assessment (LCA) followed the four LCA phases in the ISO 14040/44 standards according to ISO, I. 14040. Environmental management-life cycle assessment-principles and framework, 235-248 (2006); and Standardization, I. O. f. Environmental management: life cycle assessment; requirements and guidelines. Vol. 14044 (ISO Geneva, Switzerland, 2006), the contents of which are fully incorporated herein by reference. The goal (step 1) was to estimate the environmental impacts of two alternative catalytic pathways (i.e. heterogeneously and homogeneously-catalyzed) for the synthesis of product 1 (1-(naphthalen-1-yl)-1H-imidazole) via an Ullmann-type C—N coupling reaction, following. Both pathways employ the same substrates (1-iodonaphthalene and 1H-imidazole), but implement different reaction conditions, including solvents (dimethyl sulfoxide or N-Methyl-2-pyrrolidone, respectively), to synthesize one kg of product 1 (the functional unit). The heterogeneous reaction employs the Cu_{g}/PCN GAC developed in this work, which the homogeneous catalytic system consisted of Cu₂O with 4,7-dimethoxy-1,10-phenanthroline as the ligand (Cu₂O+L₁, Table 2). A cradle-to-gate scope was adopted, i.e., the LCA covers all the emissions associated with the upstream activities in the synthesis of substrates, solvents, and catalysts for both systems until the generation of product 1, while omitting its posterior use and disposal stages (which would be the same in both cases). No recycling was assumed for the reaction mixture in either of the routes in the baseline scenarios. However, the effect of reusing the catalyst and solvent (i.e., reducing the required amounts of these components) was explored in a sensitivity analysis considering up to 100 cycles (FIGS. 36A and 36B).

In the life cycle inventory (LCI, step two), the study used experimental values complemented with mass balances based on stoichiometric coefficients of upstream synthesis steps to estimate the whole range of inputs in the foreground system. In essence, mass balances were applied recursively in the synthesis tree of the two catalysts and one of the reactants (i.e., 1-iodonaphthalene) until reaching compounds available in ecoinvent v3.9 according to Wernet, G. et al. The ecoinvent database version 3 (part 1): overview and methodology. The International Journal of Life Cycle Assessment 21, 1218-1230 (2016), the contents of which are fully incorporated herein by reference, from where background data of the surrounding activities providing inputs to the foreground system were retrieved using the cut-off modeling choice (more details in https://ecoinvent.org/the-ecoinvent-database). In the life cycle impact assessment phase (LCIA, step three), the study computed the 100-year Global Warming Potential (GWP100), measured in kg of equivalents (kg_{CO2-eq}), considering short-lived climate forcers (SLCFs). To this end, the study followed the Intergovernmental Panel on Climate Change of the United Nations (IPCC2021) according to Forster, P. et al. The Earth's energy budget, climate feedbacks, and climate sensitivity. (2021), the contents of which are fully incorporated herein by reference, which estimates the potential of climate change induced by greenhouse gases emissions (GHGs). Moreover, the study complement the impact analysis with the endpoints of the ReCiPe2016 according to Huijbregts, M. A. et al. ReCiPe2016: a harmonised life cycle impact assessment method at midpoint and endpoint level. The International Journal of Life Cycle Assessment 22, 138-147 (2017), the contents of which are fully incorporated herein by reference, assessing the damage on three main areas of protection; human health, ecosystems quality, and resource availability. Finally, in the fourth LCA phase, the results were interpreted, identifying hotspots and providing conclusions and recommendations. All the LCA calculations were implemented in Brightway2 v2.4.1 according to Mutel, C. Brightway: an open source framework for life cycle assessment. Journal of Open Source Software 2, 236 (2017), the contents of which are fully incorporated herein by reference.

### 7.15. Assumptions and Limitations of the LCA Approach

The LCA relies on a set of assumptions, described below, which the inventors believe would not affect the main conclusions.
1. The study omitted the utilities required in the reaction steps in the foreground system. Exothermic reactions would likely require cooling water to control the reactor temperature, whose impact tends to be negligible (particularly when cooling water is recycled), while endothermic reactions would need steam. The study also note that heat integration is very challenging in batch processes, which would be most likely the preferred choice for this reaction system. Hence, it would be hard to use the heat generated in exothermic reactions to obtain environmental credits, omitted in our analysis.
2. The study further assumes that the final product could be easily separated from the reaction mixture without any energy penalty. In practice, the study should scale up the separation step using suitable unit operations. However, this separation was not attempted at the lab scale, so the study lack an experimental basis for the scale-up calculations. The study notes that both routes would require such a separation step, so it is likely that the associated impacts would be similar and the comparative assessment would lead to the same conclusions. Moreover, the homogenous system may require an additional step to separate the catalyst from the reaction mixture, which is also omitted, thereby further underestimating its impact.
3. Similarly, the study omitted the separations needed in the different synthesis steps for the catalyst components and the 1-iodonaphthalene in the foreground system. Note that the study adopted the same simplification, i.e., impacts of separations are omitted, in the synthesis of both catalytic systems, underestimating the impact in both cases.
4. The study considers that the data in the background system retrieved from ecoinvent accurately describe the life cycle activities linked to the catalytic systems. To this end, the study selected global markets representing average data worldwide. This is a common assumption in many studies that adopt the same temporal and technological level of representation as in the database.

Overall, the study provides a lower bound on the total impact for each case (i.e., an optimistic estimate), since some impact contributions were neglected, e.g., utilities and impacts of the separation steps. However, the study expect that the estimates will be close to the actual total impacts, since these are often dominated by the raw materials (explicitly considered in our LCA), as occurs in the heterogeneous case, while the other contributions tend to be significantly lower. Moreover, the same assumptions were applied in both catalytic systems, so the potential errors of the approximations may cancel out, thereby leading to similar relative performance.

It will be appreciated by a person skilled in the art that other variations and/or modifications may be made to the embodiments disclosed herein without departing from the spirit or scope of the disclosure as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

1. A material comprising a plurality of geminal metal atom pairs supported on a polymeric carrier;
wherein each geminal metal atom pair comprises two single metal atoms that are not bonded to each other; and
wherein the two single metal atoms are each bound to different metal coordination site(s) of the polymeric carrier.

2-3. (canceled)

4. The material of claim 1, wherein an average distance between the two single metal atoms is from 1.0 Å to 10.0 Å.

5. The material of claim 1, wherein each single metal atom is diagonally bound to two different metal coordination sites of the polymeric carrier.

6. The material of claim 1, wherein the material comprises a structure represented by general formula (1); and a structure represented by general formula (2):
-A¹-M¹-A²- (1)
-A³-M²-A⁴- (2)
wherein
M¹ and M² are each independently a single metal atom;
M¹ and M² both form a pair of geminal metal atoms; and
A¹, A², A³ and A⁴ are each independently a metal coordination site of the polymeric carrier.

7. The material of claim 1, wherein the material comprises from 0.1 wt % to 50.0 wt % of metal.

8. The material of claim 1, wherein the metal comprises transition metal selected from the group consisting of copper (Cu), zinc (Zn), nickel (Ni), cobalt (Co), iron (Fe), manganese (Mn), chromium (Cr), vanadium (V), titanium (Ti), scandium (Sc), yttrium (Y), zirconium (Zr), niobium (Nb), molybdenum (Mo), technetium (Tc), ruthenium (Ru), rhodium (Rh), palladium (Pd), silver (Ag), cadmium (Cd), lanthanum (La), hafnium (Hf), tantalum (Ta), tungsten (W), rhenium (Re), osmium (Os), iridium (Ir), platinum (Pt), gold (Au), mercury (Hg), actinium (Ac), and combinations thereof.

9. The material of claim 1, wherein the metal is monovalent.

10. The material of claim 1, wherein the polymeric carrier comprises an organic polymer comprising one or more atoms selected from the group consisting of carbon, nitrogen, phosphorus, sulfur, and combinations thereof.

11. The material of claim 1, wherein the polymeric carrier comprises polymeric chains having two or more fused triazine rings.

12. The material of claim 1, wherein the material comprises a first shell comprising the metal atom and a first atom of the polymeric carrier; and
wherein the interatomic distance between the metal atom and the first atom of the polymeric carrier in the first shell is from 1.0 Å to 5.0 Å.

13. (canceled)

14. The material of claim 1, wherein the material comprises a second shell comprising the metal atom and a second atom of the polymeric carrier; and
wherein the interatomic distance between the metal atom and the second atom of the polymeric carrier in the second shell is from 1.5 Å to 5.0 Å.

15. (canceled)

16. A catalyst comprising the material of claim 1.

17. A method of catalyzing coupling reactions, the method comprising:
(a-i) providing the material of claim 1; and
(a-ii) mixing said material with two or more reactants to be coupled to obtain a coupled product.

18. The method of claim 17, wherein the coupling reactions comprise cross-coupling reaction, cross-coupling bond formation, cycloaddition, carbon-carbon bond formation, carbon-heteroatom bond formation, N-arylation or combinations thereof.

19. The method of claim , 17, wherein the reactants are selected from the group consisting of heteroaryl halides, aryl halides, substrates containing hydroxyl group(s), substrates containing amino group(s), substrates containing amide group(s), substrates containing sulfonamide group(s), substrates containing alkyne group(s), substrates containing thiol group(s), substrates containing electron rich aryl group(s), substrates containing electron deficient aryl group(s), alcohols, halogenated alcohols, deuterated alcohols, heterocyclic compounds, alkynes, thiols, azides, acetylenes, L-menthol, DL-menthol, stigmasterol, DL-isoborneol, galactose, 1,2,3,4-diacetone galactose, testosterone, prednisolone, derivatives thereof and combinations thereof.

20. A method of preparing the material of claim 1, the method comprising:
(i) reacting a metal source with a polymeric carrier to obtain the material; and
(ii) isolating the material.

21. The method of claim 20, wherein the method further comprises a step of exfoliating the material into nanostructures.

22. The method of claim 20, wherein the metal source comprises a metal salt selected from the group consisting of metal chloride, metal fluoride, metal bromide, metal iodide, metal oxide, metal sulfate, metal sulfide, metal cyanide, metal thiocyanate, metal acetylide, metal acetate, metal hydroxide, metal hydride, metal nitrate, metal phosphide, metal phosphate, metal carbonate, metal carbonate hydroxide, metal chlorate, metal arsenate, metal azide, metal acetylacetonate, metal perchlorate, metal triflate, metal tetrafluoroborate, metal benzoate, metal chromite, metal peroxide, and combinations thereof.

23. The method of claim 20, wherein the metal source comprises a divalent metal cation.

24. The method of claim 20, wherein the reacting step comprises adding from 0.1 wt % to 50.0 wt % of the metal source and/or adding from 50.0 wt % to 99.9 wt % of the polymeric carrier.

25. (canceled)
